(19) United States
(12) Patent Application Publication
(10) Pub. No.: US 2024/0082369 A1
(43) Pub. Date: Mar. 14, 2024

(54) IMPROVED RECEPTOR-BINDING DOMAIN OF BOTULINUM NEUROTOXIN A AND USES THEREOF

(71) Applicant: Children's Medical Center Corporation, Boston, MA (US)

(72) Inventors: Min Dong, Weatogue, CT (US); Pyung-Gang Lee, Boston, MA (US)

(73) Assignee: Children's Medical Center Corporation, Boston, MA (US)

(21) Appl. No.: 18/268,477

(22) PCT Filed: Dec. 20, 2021
(86) PCT No.: PCT/US2021/064332

§ 371 Date: Jun. 20, 2023

Related U.S. Application Data
(60) Provisional application No. 63/128,758, filed on Dec. 21, 2020.

(51) Int. Cl. A61K 38/48; A61K 8/66; A61Q 19/00; C12N 9/52

(52) U.S. Cl. CPC A61K 38/4893; A61K 8/66; A61Q 19/00; C12N 9/52; C12Y 304/24069

(57) ABSTRACT

Disclosed herein are modified Clostridial Botulinum neurotoxin (BoNT) polypeptides with a modified receptor binding domain of Clostridial Botulinum serotype A1 or A2. Modifications include substitution amino acid mutations. Isolated modified receptor binding domains, chimeric molecules, pharmaceutical compositions, and methods of using the same are also disclosed.

### RELATED APPLICATIONS

This application claims the benefit under 35 U.S.C. § 119(e) of U.S. provisional application No. 63/128,758, titled “IMPROVED RECEPTOR-BINDING DOMAIN OF BOTULINUM NEUROTOXIN A AND USES THEREOF,” filed Dec. 21, 2020, the entire contents of which are incorporated herein by reference.

### FEDERALLY SPONSORED RESEARCH

This invention was made with government support under Grant No: NS080833 awarded by the National Institutes of Health. The government has certain rights in the invention.

### BACKGROUND

In recent years, Clostridial Botulinum neurotoxin (BoNT) have been widely used to treat a growing list of medical conditions: local injections of minute amount of toxins can attenuate neuronal activity in targeted regions, which can be beneficial in many medical conditions as well as for cosmetic purposes. To date, BoNT serotype A (BoNT/A) and BoNT serotype B (BoNT/B) are the only two BoNTs that are currently FDA-approved for use in humans. The major limitation is the generation of neutralizing antibodies in patients, which renders future treatment ineffective. Termination of BoNT usage often leaves patients with no other effective ways to treat/relieve their disorders. Adverse effects associated with BoNT use range from transient nonserious events such as ptosis and diplopia to life-threatening events even death.

### SUMMARY

The limitations and adverse effects of BoNTs are largely correlated with dose. Provided herein are modified BoNTs with improved activity. The modified BoNTs described herein maintain the same level of toxin activity with lower dose, thus reducing the possibility of the generation of neutralizing antibodies in patients and adverse side effects.

Some aspects of the present disclosure provide modified Clostridial Botulinum neurotoxin (BoNT) polypeptides comprising a modified receptor binding domain of Clostridial Botulinum serotype A1 (BoNT/A1) or a modified receptor binding domain of Clostridial Botulinum serotype A1 (BoNT/A2). In some aspects, the present disclosure provides modified receptor binding domains of BoNT/A1 or BoNT/A2. In some aspects, the present disclosure provides chimeric BoNT polypeptides comprising a modified receptor binding domain of BoNT/A1 or BoNT/A2. In some embodiments, a BoNT comprising the modified receptor binding domain of a BoNT/A1 or BoNT/A2 increases binding of the BoNT to a BoNT receptor protein compared to a BoNT comprising a wildtype BoNT/A1 or BoNT/A2 receptor binding domain. In some embodiments, a BoNT comprising the modified receptor binding domain of BoNT/A1 or BoNT/A2 reduces systemic toxicity at a dosage that induces the same degree of paralysis as a BoNT comprising a wildtype BoNT/A1 or BoNT/A2 receptor binding domain.

In some embodiments, a BoNT comprising the modified receptor binding domain of a BoNT/A1 or BoNTA2 increases local paralysis compared to a BoNT comprising a wildtype BoNT/A receptor binding domain. In some embodiments, local paralysis induced by a BoNT comprising a modified BoNT/A1 or BoNT/A2 receptor binding domain is quantified using a Digital Abduction Score (DAS) assay. In some embodiments, the BoNT comprising a modified BoNT/A1 or BoNT/A2 receptor binding domain has an increased DAS score as compared by a wildtype BoNT. In some embodiments, the DAS score of BoNT comprising a modified BoNT/A1 or BoNT/A2 receptor binding domain is greater than the DAS Score of a wildtype BoNT by 0.5, 1, 2, 3 or 4. In some embodiments, the DAS score of BoNT comprising a modified BoNT/A1 or BoNT/A2 receptor binding domain is greater than the DAS Score of a wildtype BoNT by at least 0.5, 1, 2, 3 or 4. In some embodiments, the BoNT comprising a modified BoNT/A1 or BoNT/A2 receptor binding domain has a DAS score of at least 1 (e.g. at least 1, at least 2, at least 3, or least 4). In some embodiments, the BoNT comprising a modified BoNT/A1 or BoNT/A2 receptor binding domain has a DAS score of 1-2, 2-3, or 3-4.

In some embodiments, the modified Clostridial Botulinum neurotoxin (BoNT) polypeptide comprises a modified receptor binding domain of Clostridial Botulinum serotype A1 (BoNT/A1) comprising one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1.

In some embodiments, the modified Clostridial Botulinum neurotoxin (BoNT) polypeptide comprises a modified receptor binding domain of Clostridial Botulinum serotype A1 (BoNT/A1) comprising one or more amino acid substitutions at positions corresponding to 954, 955, 957, 1063, 1064, 1025, 1026, 1156, 1232, 1278, 1294, and 1295 in SEQ ID NO: 1.

In some embodiments, the modified Clostridial Botulinum neurotoxin (BoNT) polypeptide comprises a modified receptor binding domain of Clostridial Botulinum serotype A1 (BoNT/A1) comprising an amino acid substitution at a position corresponding to 917 in SEQ ID NO: 1, optionally wherein the amino acid substitution corresponds to F917R or F917K in SEQ ID NO: 1.

In some embodiments, the modified receptor binding domain of the modified BoNT/A1 polypeptide comprises an amino acid substitution at a position corresponding to 953 in SEQ ID NO: 1, optionally wherein the amino acid substitution corresponds to F953H or F953Y in SEQ ID NO: 1.

In some embodiments, the modified receptor binding domain of the modified BoNT/A1 polypeptide comprises an amino acid substitution at a position corresponding to 954 in SEQ ID NO: 1, optionally wherein the amino acid substitution corresponds to N954S in SEQ ID NO: 1.

In some embodiments, the modified receptor binding domain of the modified BoNT/A1 polypeptide comprises an amino acid substitution at a position corresponding to 955 in SEQ ID NO: 1, optionally wherein the amino acid substitution corresponds to S955K in SEQ ID NO: 1.

In some embodiments, the modified receptor binding domain of the modified BoNT/A1 polypeptide comprises an amino acid substitution at a position corresponding to 957 in SEQ ID NO: 1, optionally wherein the amino acid substitution corresponds to S957N, S957Q, or S957Y in SEQ ID NO: 1.

In some embodiments, the modified receptor binding domain of the modified BoNT/A1 polypeptide comprises an amino acid substitution at a position corresponding to 968 in SEQ ID NO: 1, optionally wherein the amino acid substitution corresponds to M968I in SEQ ID NO: 1.

In some embodiments, the modified receptor binding domain of the modified BoNT/A1 polypeptide comprises an amino acid substitution at a position corresponding to 1025 in SEQ ID NO: 1, optionally wherein the amino acid substitution corresponds to N1025T in SEQ ID NO: 1.

In some embodiments, the modified receptor binding domain of the modified BoNT/A1 polypeptide comprises an amino acid substitution at a position corresponding to 1026 in SEQ ID NO: 1, optionally wherein the amino acid substitution corresponds to N1026K in SEQ ID NO: 1.

In some embodiments, the modified receptor binding domain of the modified BoNT/A1 polypeptide comprises an amino acid substitution at a position corresponding to 1052 in SEQ ID NO: 1, optionally wherein the amino acid substitution corresponds to N1052K in SEQ ID NO: 1.

In some embodiments, the modified receptor binding domain of the modified BoNT/A1 polypeptide comprises an amino acid substitution at a position corresponding to 1062 in SEQ ID NO: 1, optionally wherein the amino acid substitution corresponds to D1062E in SEQ ID NO: 1.

In some embodiments, the modified receptor binding domain of the modified BoNT/A1 polypeptide comprises an amino acid substitution at a position corresponding to 1063 in SEQ ID NO: 1, optionally wherein the amino acid substitution corresponds to T1063P in SEQ ID NO: 1.

In some embodiments, the modified receptor binding domain of the modified BoNT/A1 polypeptide comprises an amino acid substitution at a position corresponding to 1064 in SEQ ID NO: 1, optionally wherein the amino acid substitution corresponds to H1064R or H1064Q in SEQ ID NO: 1.

In some embodiments, the modified receptor binding domain of the modified BoNT/A1 polypeptide comprises an amino acid substitution at a position corresponding to 1065 in SEQ ID NO: 1, optionally wherein the amino acid substitution corresponds to R1065N in SEQ ID NO: 1.

In some embodiments, the modified receptor binding domain of the modified BoNT/A1 polypeptide comprises an amino acid substitution at a position corresponding to 1066 in SEQ ID NO: 1, optionally wherein the amino acid substitution corresponds to Y1066R or Y1066K in SEQ ID NO: 1.

In some embodiments, the modified receptor binding domain of the modified BoNT/A1 polypeptide comprises an amino acid substitution at a position corresponding to 1145 in SEQ ID NO: 1, optionally wherein the amino acid substitution corresponds to T1145Y in SEQ ID NO: 1.

In some embodiments, the modified receptor binding domain of the modified BoNT/A1 polypeptide comprises an amino acid substitution at a position corresponding to 1156 in SEQ ID NO: 1, optionally wherein the amino acid substitution corresponds to R1156M or R1156I in SEQ ID NO: 1.

In some embodiments, the modified receptor binding domain of the modified BoNT/A1 polypeptide comprises an amino acid substitution at a position corresponding to 1232 in SEQ ID NO: 1, optionally wherein the amino acid substitution corresponds to T1232R or T1232K in SEQ ID NO: 1.

In some embodiments, the modified receptor binding domain of the modified BoNT/A1 polypeptide comprises an amino acid substitution at a position corresponding to 1272 in SEQ ID NO: 1, optionally wherein the amino acid substitution corresponds to E1272G in SEQ ID NO: 1.

In some embodiments, the modified receptor binding domain of the modified BoNT/A1 polypeptide comprises an amino acid substitution at a position corresponding to 1278 in SEQ ID NO: 1, optionally wherein the amino acid substitution corresponds to L1278F, L1278Y, or L1278W in SEQ ID NO: 1.

In some embodiments, the modified receptor binding domain of the modified BoNT/A1 polypeptide comprises an amino acid substitution at a position corresponding to 1288 in SEQ ID NO: 1, optionally wherein the amino acid substitution corresponds to D1288E or D1288N in SEQ ID NO: 1.

In some embodiments, the modified receptor binding domain of the modified BoNT/A1 polypeptide comprises an amino acid substitution at a position corresponding to 1289 in SEQ ID NO: 1, optionally wherein the amino acid substitution corresponds to D1289Y in SEQ ID NO: 1.

In some embodiments, the modified receptor binding domain of the modified BoNT/A1 polypeptide comprises an amino acid substitution at a position corresponding to 1292 in SEQ ID NO: 1, optionally wherein the amino acid substitution corresponds to G1292R or G1292K in SEQ ID NO: 1.

In some embodiments, the modified receptor binding domain of the modified BoNT/A1 polypeptide comprises an amino acid substitution at a position corresponding to 1294 in SEQ ID NO: 1, optionally wherein the amino acid substitution corresponds to R1294S or R1294T in SEQ ID NO: 1.

In some embodiments, the modified receptor binding domain of the modified BoNT/A1 polypeptide comprises an amino acid substitution at a position corresponding to 1295 in SEQ ID NO: 1, optionally wherein the amino acid substitution corresponds to P1295S or P1295T in SEQ ID NO: 1.

In some embodiments, the modified receptor binding domain of the modified BoNT/A1 polypeptide comprises an amino acid sequence that is at least 80% identical to the amino acid sequence of SEQ ID NO: 51-85

In some embodiments, the modified receptor binding domain of the modified BoNT/A1 polypeptide comprises the amino acid sequence of any one of SEQ ID NOs:51-85.

In some embodiments, the modified receptor binding domain of the modified BoNT/A1 polypeptide further comprises a protease domain and a translocation domain from BoNT/A1.
In some embodiments, the modified BoNT/A1 polypeptide comprises an amino acid sequence that is at least 80% identical to the amino acid sequence of SEQ ID NO: 3-37.
In some embodiments, the modified BoNT/A1 polypeptide comprises the amino acid sequence of SEQ ID NO: 3-37.

In some embodiments, the modified BoNT/A1 polypeptide comprises a protease domain and a translocation domain from a second BoNT optionally wherein the second BoNT is of serotype B, C, D, E, F, G, H, X, or En.

In some embodiments, the modified BoNT/A1 polypeptide comprising a protease domain and a translocation domain from a second BoNT comprises an amino acid sequence that is at least 80% identical to the amino acid sequence of any one of SEQ ID NO: 97-105 fused to any one of SEQ ID NO: 51-85.

In some embodiments, the modified BoNT/A1 polypeptide comprising a protease domain and a translocation domain from a second BoNT comprises the amino acid sequence of any one of SEQ ID NO: 97-105 fused to any one of SEQ ID NO: 51-85.

In some embodiments, the modified Clostridial Botulinum neurotoxin (BoNT) polypeptide comprises a modified receptor binding domain of Clostridial Botulinum serotype A2 (BoNT/A2) comprising one or more amino acid substitutions at positions corresponding to 915, 923, 1090, 1103, 1117, 1156, 1170, 1227, 1254, 1255, or 1256 in SEQ ID NO: 2.

In some embodiments, the modified receptor binding domain of the modified BoNT/A2 polypeptide comprises an amino acid substitution at a position corresponding to 915 in SEQ ID NO: 2, optionally wherein the amino acid substitution corresponds to K915Q in SEQ ID NO: 2.

In some embodiments, the modified receptor binding domain of the modified BoNT/A2 polypeptide comprises an amino acid substitution at a position corresponding to 923 in SEQ ID NO: 2, optionally wherein the amino acid substitution corresponds to T923K in SEQ ID NO: 2.

In some embodiments, the modified receptor binding domain of the modified BoNT/A2 polypeptide comprises an amino acid substitution at a position corresponding to 1090 in SEQ ID NO: 2, optionally wherein the amino acid substitution corresponds to S1090N in SEQ ID NO: 2.

In some embodiments, the modified receptor binding domain of the modified BoNT/A2 polypeptide comprises an amino acid substitution at a position corresponding to 1103 in SEQ ID NO: 2, optionally wherein the amino acid substitution corresponds to N1103D in SEQ ID NO: 2.

In some embodiments, the modified receptor binding domain of the modified BoNT/A2 polypeptide comprises an amino acid substitution at a position corresponding to 1117 in SEQ ID NO: 2, optionally wherein the amino acid substitution corresponds to F1117Y in SEQ ID NO: 2.

In some embodiments, the modified receptor binding domain of the modified BoNT/A2 polypeptide comprises an amino acid substitution at a position corresponding to 1156 in SEQ ID NO: 2, optionally wherein the amino acid substitution corresponds to E1156M in SEQ ID NO: 2.

In some embodiments, the modified receptor binding domain of the modified BoNT/A2 polypeptide comprises an amino acid substitution at a position corresponding to 1170 in SEQ ID NO: 2, optionally wherein the amino acid substitution corresponds to E1170K in SEQ ID NO: 2.

In some embodiments, the modified receptor binding domain of the modified BoNT/A2 polypeptide comprises an amino acid substitution at a position corresponding to 1227 in SEQ ID NO: 2, optionally wherein the amino acid substitution corresponds to D1227N in SEQ ID NO: 2.

In some embodiments, the modified receptor binding domain of the modified BoNT/A2 polypeptide comprises an amino acid substitution at a position corresponding to 1254 in SEQ ID NO: 2, optionally wherein the amino acid substitution corresponds to L1254Q in SEQ ID NO: 2.

In some embodiments, the modified receptor binding domain of the modified BoNT/A2 polypeptide comprises an amino acid substitution at a position corresponding to 1255 in SEQ ID NO: 2, optionally wherein the amino acid substitution corresponds to Y1255F in SEQ ID NO: 2.

In some embodiments, the modified receptor binding domain of the modified BoNT/A2 polypeptide comprises an amino acid substitution at a position corresponding to 1256 in SEQ ID NO: 2, optionally wherein the amino acid substitution corresponds to D1256N in SEQ ID NO: 2.

In some embodiments, the modified receptor binding domain of the modified BoNT/A2 polypeptide comprises an amino acid sequence that is at least 80% identical to the amino acid sequence of SEQ ID NO: 86-96.

In some embodiments, the modified receptor binding domain of the modified BoNT/A2 polypeptide comprises the amino acid sequence of any one of SEQ ID NOs: 86-96

In some embodiments, the modified BoNT/A2 polypeptide further comprises a protease domain and a translocation domain from BoNT/A1. In some embodiments, the modified BoNT/A2 polypeptide comprises an amino acid sequence that is at least 80% identical to the amino acid sequence of SEQ ID NO: 38-48. In some embodiments, the modified BoNT/A2 polypeptide comprises the amino acid sequence of SEQ ID NO: 38-48. In some embodiments, the modified BoNT/A2 polypeptide, further comprises a protease domain and a translocation domain from a second BoNT, optionally wherein the second BoNT is of serotype B, C, D, E, F, G, H, X, or En. In some embodiments, the modified BoNT/A2 polypeptide comprising a protease domain and a translocation domain from a second BoNT comprises an amino acid sequence is at least 80% identical to the amino acid sequence of any one of SEQ ID NO: 97-105 fused to any one of SEQ ID NO: 86-96. In some embodiments, the modified BoNT/A2 polypeptide comprising a protease domain and a translocation domain from a second BoNT comprises the amino acid sequence of any one of SEQ ID NO: 97-105 fused to any one of SEQ ID NO: 86-96.

In certain aspects, a nucleic acid molecule comprises a polynucleotide encoding any one of the modified BoNT polypeptides, modified receptor binding domains, or chimeric BoNTs described herein. In some embodiments, a nucleic acid vector comprises the nucleic acid molecule comprising the polynucleotide encoding any one of the modified BoNT polypeptides, modified receptor binding domains, or chimeric BoNTs described herein. In some embodiments, a cell comprises the nucleic acid molecule or the nucleic acid vector encoding any one of the modified BoNT polypeptides, modified receptor binding domains, or chimeric BoNTs described herein.

In some aspects the present disclosure provides methods of producing a modified BoNT polypeptide comprising the steps of culturing the cell comprising the nucleic acid molecule or the nucleic acid vector encoding any one of the modified BoNT polypeptides, modified receptor binding domains, or chimeric BoNTs described herein under conditions wherein the modified BoNT polypeptide is produced. In some embodiments, the method of producing a modified BoNT polypeptide further comprises recovering the modified BoNT polypeptide from the culture.

In some aspects, a pharmaceutical composition comprises the modified BoNT polypeptide of any one of the modified BoNT polypeptides, the modified receptor binding domains, or the chimeric BoNTs described herein or a nucleic acid or nucleic acid vector encoding any one of the modified BoNT polypeptides, modified receptor binding domains, chimeric BoNTs described herein. In some embodiments, the pharmaceutical composition further comprises a pharmaceutically acceptable excipient.

In some aspects, a kit comprising a pharmaceutical composition described herein is provided with directions for therapeutic administration of the pharmaceutical composition. In some aspects, the disclosure provides a method of treating a condition, the method comprising administering a therapeutically effective amount of the modified BoNT polypeptide, modified BoNT receptor binding domain or a modified Chimeric BoNT described herein, or the pharmaceutical compositions described herein to a subject to treat the condition. In some embodiments, the condition to be treated is associated with overactive neurons or glands.

In some embodiments, the condition is selected from the group consisting of: spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite, depression.

In some embodiments, the condition is not associated with unwanted neuronal activity. In some embodiments, the condition is selected from the group consisting of: psoriasis, allergy, haemophagocytic lymphohistiocytosis, and alcoholic pancreatic disease.

In some embodiments, treating a condition comprises administering via injection to where unwanted neuronal activity is present.

Other aspects of the present disclosure provide use of the modified BoNT polypeptide, modified BoNT receptor binding domain or a modified Chimeric BoNT, or the pharmaceutical compositions described herein treating a condition associated with unwanted neuronal activity.

Further provided herein are uses of the modified BoNT polypeptide, modified BoNT receptor binding domain or a modified Chimeric BoNT, or the pharmaceutical compositions described herein in medicine.

Further provided herein are cosmetic uses of the modified BoNT polypeptide, modified BoNT receptor binding domain or a modified Chimeric BoNT, or the pharmaceutical compositions described herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show the schematic view of BoNT action and its domains: (FIG. 1A) A schematic view of BoNT actions: BoNTs recognize neurons by binding to their specific receptors, enter neurons via receptor-mediated endocytosis, the light chains of BoNTs then translocate across endosomal membranes into the cytosol, where these light chains act as proteases to cleave target host proteins. FIG. 1A is adapted from Arnon, S. et al, JAMA, 285:1059, 2001 31. (FIG. 1B) BoNTs are composed of a light chain and a heavy chain, connected via a disulfide bond. The heavy chain can be further divided into two domains: the translocation domain (H_{N}) and the receptor binding domain (H_{C}).

FIGS. 2A-2B show the sequence alignment of BoNT/A subtypes and the selected mutation sites: (FIG. 2A) Sequence alignment of the H_{C}s of BoNT/A1-A8 and BoNT/H, with the selected consensus mutation site noted with boxes. (FIG. 2B) Crystal structure of H_{C}/A1 with targeted mutagenesis sites labeled.

FIG. 3 shows H_{C}/A-SV2 complex structure with selected mutagenesis sites labeled for enhancing binding to glycosylated SV2: Crystal structure of H_{C}/A1 complexed with glycosylated SV2C-LD4 (PDB: 5JLV) are shown with glycan moiety. The residues involved in glycan binding are marked as black with residue label.

FIG. 4A-4F show sortase-mediated ligation of toxins: (FIG. 4A) A schematic drawing of sortase ligation: A1-LC-H_{N} has a sortase tag (LPETG) on its C-terminus which can be ligated with N-terminal free glycine of A1-H_{C} by sortase. After ligation, the full-length protein should be activated by thrombin to cleave a linker between LC and H_{N} to obtain its functional activity. (FIGS. 4B-4F) SDS-PAGE gel image showing the ligation and activation. The full-length ligated toxins (fl-BoNT/A) can be shown at ˜150 kDa without treatment of 2-mercaptoethanol (2-ME), but separated into LC and HC with 2-ME. All the mutant toxins tested in this study were ligated and analyzed using this method.

FIG. 5A-5E show evaluation of mutant toxins in vivo using DAS assays: (FIG. 5A) A standard scoring for DAS assay. Score 0 means normal, while score 4 represents most severe paralysis. (FIG. 5B) DAS results of H_{C}/A1 mutants: NSIS954-957SKIN, TQEIK990-994NKQNI, T1063P/H1064R and L1278F along with H_{C}/A1-WT (control). These mutants showed similar or reduced activity compared to WT. The most detrimental mutations were T1063P/H1064R. (FIG. 5C) DAS results of H_{C}/A1 mutants: F917R, F917K, N954Q, S957Q and D1289Y along with H_{C}/A1-WT (control). These mutants target glycan binding sites of the toxin. All tested 5 mutants showed reduced activity compared to WT, while S957Q showed no paralysis. (FIG. 5D) DAS results of H_{C}/A1 mutants: N1025T/N1026K, R1156M, T1232R, and R1294S/P1295S along with H_{C}/A1-WT (control). These mutants all showed significantly higher paralysis than WT. (FIG. 5E) DAS results of H_{C}/A1 mutants: T1063P and H1064Q along with H_{C}/A1-WT. T1063P showed higher scores than WT, while H1064Q showed reduced activity compared to WT.

### DETAILED DESCRIPTION OF CERTAIN EMBODIMENTS

Clostridium botulinum neurotoxins (BoNTs) are a family of bacterial toxins produced by Clostridium bacteria, with seven well-established serotypes (BoNT/A-G) ^{1, 32-33} and two recently discovered serotypes X and EN described in U.S. patent application Ser. Nos. 16/315,698 and 16/651,720 (both incorporated herein by reference). They are one of the most dangerous potential bio-terrorism agents, classified as a “Category A” select agent by Center for Disease Control (CDC) of United States ³¹. These toxins are produced as a single polypeptide and can be separated by bacterial or host proteases into a light chain (LC, ˜50 kDa) and a heavy chain (HC, ˜100 kDa). The two chains remain connected via an inter-chain disulfide bond. The HC contains two sub-domains: the N-terminal H_{N} domain that mediates translocation of the LC across endosomal membranes, and the C-terminal H_{C} domain that mediates binding to receptors on neurons. The inter-chain disulfide bond is reduced once the LC translocates into the cytosol ³⁴⁻³⁵. Released LC acts as a protease to specifically cleave a set of neuronal proteins: BoNT/A, C, and E cleave at distinct sites on a protein known as SNAP-25; BoNT/B, D, F, and G cleave at different sites on a vesicle protein VAMP; and BoNT/C also cleaves a transmembrane protein syntaxin 1 ^{1, 32-33}. These three proteins form a complex, known as SNARE complex, which is essential for release of neurotransmitters ³⁶⁻³⁷. Cleavage of any one of these three SNARE proteins blocks neurotransmitters release from neurons, thus paralyzing muscles. Recently discovered BoNT/X cleaves SNARE proteins like other BoNTs, but also cleaves non-canonical substrates VAMP4, VAMP5 and Ykt6. Recently discovered BoNT/EN cleaves VAMP1/2/3 and several other SNARE proteins including SNAP-25, SNAP-23, syntaxin 1B and syntaxin 4.

BoNTs are the most potent toxins known and cause the human and animal disease known as botulism ³³. The major form of botulism is caused by ingesting food contaminated with BoNTs (food botulism). Other forms also exist such as infant botulism, which is due to colonization of the intestine by toxin-producing bacteria in infants. BoNTs are always produced together with another 150 kDa protein known as NTNHA (non-toxic non-hemagglutinin protein), which forms a pH-dependent complex with BoNTs and protects BoNTs from proteases in the gastrointestinal tract ³⁸.

Because local injections of minute amounts of toxins can attenuate neuronal activity in targeted regions, BoNTs have been used to treat a growing list of medical conditions ³⁻⁵, including muscle spasms, chronic pain, overactive bladder problems, as well as for cosmetic applications. The market for BoNTs has already surpassed $3 billion in 2018. Among the seven types of BoNT toxins, BoNT/A and BoNT/B are the two toxins that are currently FDA-approved for use in humans ³⁻⁵. BoNT/A is the dominant type used for both medical and cosmetic applications, marketed as Botox from Allergan Inc., Dysport from IPSEN Inc., and Xeomin from Merz Inc. BoNT/B is marketed as Myobloc by USWorld Med.

As the application of BoNTs grows, limitations and adverse effects have been reported. The major limitation is the generation of neutralizing antibodies in patients, which renders future treatment ineffective ⁶. Termination of BoNT usage often leaves patients with no other effective ways to treat or relieve their disorders. The probability of antibody responses is directly related to both toxin doses and the frequency of injection ⁶. Therefore, this limitation mainly occurs in treating muscle spasms, which involves high dose of toxins. Consistently, antibody responses have not been observed in cosmetic applications, which use extremely low toxin doses ⁶.

The major adverse effects are also often associated with treating muscle spasms, but not cosmetic applications. This is because the adverse effects are largely due to diffusion of toxins to other regions of the body and the possibility of toxin diffusion is directly related to injected doses. The adverse effects range from transient non-serious events such as ptosis and diplopia to life-threatening events even death ^{7, 8}. In a petition letter filed in 2008 by Dr. Sidney Wolfe to FDA, a total of 180 serious adverse events, including 16 deaths have been documented. As a result, FDA now requires the “Black box warning” on all BoNT products, highlighting the risk of the spread of toxins, following similar warnings issued by the European Union.

Because both the generation of neutralizing antibodies and toxin diffusion are directly related to injected doses, lowering toxin doses (while maintaining the same levels of toxin activity) is highly desired, which means the efficacy of individual toxin molecules to induce local muscle paralysis has to be enhanced. Such modified BoNTs with improved local efficacy would also reduce any potential off-target effects due to toxin diffusion to other regions.

The action of BoNTs has three major steps: (1) receptor binding: these toxins target motor nerve terminals by first binding specifically to their receptors expressed in neurons; (2) translocation: after binding to receptors, BoNTs enter cells via receptor-mediated endocytosis into endosomes, and the low pH conditions within endosomes then induce conformational changes of toxin, resulting in its penetration of endosomal membrane and release of its protease domain into the cytosol of neurons; (3) substrate cleavage: within the cytosol of neurons, the released protease domain of BoNTs then cleave proteins required for synaptic transmission, therefore blocking neurotransmission ². Corresponding to these three steps of action, BoNTs are composed of three functional domains ²: (1) the C-terminal receptor-binding domain (H_{C}, ˜50 kDa); (2) the membrane translocation domain in the middle (H_{N}, ˜50 kDa); (3) the N-terminal protease domain (also known as light chain, LC, ˜50 kDa). The H_{C} and H_{N} together form the heavy chain (HC, ˜100 kDa).

Receptor-binding appears to be a rate-limiting step. For instance, enhancing the ability of BoNTs to recognize their neuronal receptors will facilitate absorbance of toxins into neurons at the injection site, therefore shielding toxins from triggering immune responses and also preventing their diffusion. Enhanced affinity and specificity to neuronal receptors will also reduce potential off-target effects due to non-specific entry into other cell types. The receptors for most BoNTs have been identified in recent years. BoNT/B, G, and a mosaic toxin DC share two homologous synaptic vesicle proteins synaptotagmin I and II (Syt I/II) as their receptors ⁹⁻¹⁶. Another family of synaptic vesicle protein SV2 acts as receptors for BoNT/A, E, D, and potentially F ^{10, 17-22}. In addition to protein receptors, all BoNTs require lipid co-receptor gangliosides, which are abundant on neuronal surfaces ²³.

Accordingly, some aspects of the present disclosure provide modified Clostridial Botulinum neurotoxins (BoNT) comprising a modified receptor binding domain of Clostridial Botulinum serotype A (BoNT/A). In some embodiments, a BoNT comprising the modified receptor binding domain of a BoNT/A increases binding of the BoNT to a BoNT receptor protein compared to a BoNT comprising a wildtype BoNT/A receptor binding domain. In some embodiments, a BoNT comprising the modified receptor binding domain of BoNT/A reduces systemic toxicity at a dosage that induces the same degree of local paralysis as a BoNT comprising a wildtype BoNT/A receptor binding domain. In some embodiments, a BoNT comprising the modified receptor binding domain of a BoNT/A increases local paralysis compared to a BoNT comprising a wildtype BoNT/A receptor binding domain.

As used herein, the term “Clostridial Botulinum neurotoxin (BoNT)” encompasses any polypeptide or fragment from a Botulinum neurotoxin. In some embodiments, the term BoNT refers to a full-length BoNT. In some embodiments, the term BoNT refers to a fragment of the BoNT that can execute the overall cellular mechanism whereby a BoNT enters a neuron and inhibits neurotransmitter release. In some embodiments, the term BoNT simply refers to a fragment of the BoNT, without requiring the fragment to have any specific function or activity. Other terms that may be used throughout the present disclosure for “Clostridial Botulinum neurotoxins” may be BoNTs, Botulinum toxins, or C. Botulium toxins. It is to be understood that these terms are used interchangeably.

A “modified Clostridial Botulinum neurotoxin (BoNT)” encompasses a BoNT comprising any modifications in the amino acid sequence, e.g., truncation, addition, amino acid substitution, and any combination thereof. For example, a BoNT/A1 comprising amino acid substitution mutation in F917 or S955 is a modified BoNT. In another example, a fragment or a domain of the full-length BoNT (e.g., the receptor binding domain) is considered a modified BoNT. In some embodiments, a domain of the BoNT may also comprise amino acid substitution mutation(s), e.g., a receptor binding domain comprising substitution mutation at positions corresponding to F917R or S955 of the full-length BoNT.

As used herein, the term “Clostridial Botulinum neurotoxin (BoNT) protease domain” means a BoNT domain that can execute the enzymatic target modification step of the intoxication process. Thus, a BoNT protease domain specifically targets a C. Botulinum toxin substrate and encompasses the proteolytic cleavage of a C. Botulinum toxin substrate, such as, e.g., SNARE proteins like a SNAP-25 substrate, a VAMP substrate and a Syntaxin substrate.

As used herein, the term “Clostridial Botulinum neurotoxin (BoNT) translocation domain” or “H_{N}” means a BoNT domain that can execute the translocation step of the intoxication process that mediates BoNT light chain translocation. Thus, an H_{N} facilitates the movement of a BoNT light chain across a membrane into the cytoplasm of a cell. Non-limiting examples of a Hn include a BoNT/A H_{N}, a BoNT/B H_{N}, a BoNT/Cl H_{N}, a BoNT/D H_{N}, a BoNT/E H_{N}, a BoNT/F H_{N}, and a BoNT/G H_{N}.

As used herein, the term “Clostridial Botulinum neurotoxin (BoNT) receptor-binding domain” is synonymous with “H_{C} domain” and “H_{C}”, and means any naturally occurring BoNT receptor binding domain that can execute the cell binding step of the intoxication process, including, e.g., the binding of the BoNT to a BoNT-specific receptor system located on the plasma membrane surface of a target cell. Some aspects of present disclosure relate to modified BoNT receptor binding domains from serotype A (BoNT/A), that enhances the binding of the BoNT/A to a cell, e.g., neurons or a BoNT/A receptor. BoNT/A has eight subtypes, BoNT/A1, BoNT/A2, BoNT/A3, BoNT/A4, BoNT/A5, BoNT/A6, BoNT/A7, and BoNT/A8. Thus, the present disclosure encompasses modified BoNT/A receptor binding domain from all and any of the eight subtypes. It is appreciated that when “BoNT/A” is referred to, it encompasses all the subtypes of BoNT/A. In some embodiments, a “modified BoNT/A receptor binding domain” comprises novel amino acid substitution mutations described in the present disclosure.

Some aspects of present disclosure relate to modified BoNT receptor binding domains from serotype A1 (BoNT/A1), that enhances the binding of the BoNT/A1 to a cell, e.g., neurons or a BoNT/A1 receptor. Thus, the present disclosure encompasses modified BoNT/A1 receptor binding domain from all and any of the eight subtypes. In some embodiments, a “modified BoNT/A1 receptor binding domain” comprises novel amino acid substitution mutations described in the present disclosure. In some embodiments, the modified receptor binding domain of BoNT/A1 comprises about amino acids 873-1296 of SEQ ID NO: 1. It is to be understood that the border of the BoNT/A1 receptor binding domain fragment may vary by 1-10 amino acids. For example, the modified BoNT/A1 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 1.

Some aspects of present disclosure relate to modified BoNT receptor binding domains from serotype A2 (BoNT/A2), that enhances the binding of the BoNT/A2 to a cell, e.g., neurons or a BoNT/A2 receptor. Thus, the present disclosure encompasses modified BoNT/A2 receptor binding domain from all and any of the eight subtypes. In some embodiments, a “modified BoNT/A2 receptor binding domain” comprises novel amino acid substitution mutations described in the present disclosure. In some embodiments, the modified receptor binding domain of BoNT/A2 comprises about amino acids 873-1296 of SEQ ID NO: 2. It is to be understood that the border of the BoNT/A2 receptor binding domain fragment may vary by 1-10 amino acids. For example, the modified BoNT/A2 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 2.

In some aspects, the modified BoNT polypeptide is a chimeric toxin, wherein protease domain and translocation domain are from a serotype selected from the group consisting of A, B, C, D, E, F, G, X or En and combinations thereof and the modified receptor binding domain comprises any one of the BoNT/A1 or BoNT/A2 modified receptor binding domains described herein. In some embodiments the protease and translocation domains may be fused with any one of the modified receptor binding domains of BoNT/A1 or BoNT/A2. In a non-limiting example, a chimeric BoNT/B|A1-F917R (nomenclature: protease and transmembrane domain from BoNT/B and modified receptor binding from BoNT/A1 with a F917R modification) may be produced by fusing the protease and transmembrane domain of a BoNT of serotype B with a modified receptor binding domain of BoNT/A1 comprising a phenylalanine to arginine mutation at position 917 of SEQ ID NO: 1. In a non-limiting example, a chimeric BoNT/C|A2-K915Q may be produced by fusing the protease and transmembrane domain of a BoNT of serotype C with a modified receptor binding domain of BoNT/A2 comprising a lysine to glutamine mutation at position 915 of SEQ ID NO: 2.

In some embodiments, the modified BoNT polypeptide is a chimeric toxin, wherein protease domain and translocation domain comprise the BoNT/B serotype. The protease and transmembrane domain of BoNT/B comprises about amino acids 1-857 of SEQ ID NO: 97. It is to be understood that the border of the BoNT/B protease and transmembrane domain may vary by 1-10 amino acids. For example, the BoNT/B protease and transmembrane domain that may be used for the chimeric toxin may comprise amino acids 1-847, 1-848, 1-849, 1-850, 1-851, 1-852, 1-853, 1-854, 1-855, 1-856, 1-857, 1-858, 1-859, 1-860, 1-861, 1-862, 1-863, 1-864, 1-865, 1-866, or 1-867 of SEQ ID NO: 97.

In some embodiments, the modified BoNT polypeptide is a chimeric toxin, wherein protease domain and translocation domain comprise the BoNT/C serotype. The protease and transmembrane domain of BoNT/C comprises about amino acids 1-870 of SEQ ID NO: 98. It is to be understood that the border of the BoNT/C protease and transmembrane domain may vary by 1-10 amino acids. For example, the BoNT/C protease and transmembrane domain that may be used for the chimeric toxin may comprise amino acids 1-860, 1-861, 1-862, 1-863, 1-864, 1-865, 1-866, 1-867, 1-868, 1-869, 1-870, 1-871, 1-872, 1-873, 1-874, 1-875, 1-876, 1-877, 1-878, 1-879, or 1-880 of SEQ ID NO: 98.

In some embodiments, the modified BoNT polypeptide is a chimeric toxin, wherein protease domain and translocation domain comprise the BoNT/D serotype. The protease and transmembrane domain of BoNT/D comprises about amino acids 1-862 of SEQ ID NO: 99. It is to be understood that the border of the BoNT/D protease and transmembrane domain may vary by 1-10 amino acids. For example, the BoNT/D protease and transmembrane domain that may be used for the chimeric toxin may comprise amino acids 1-852, 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, 1-872 of SEQ ID NO: 99.

In some embodiments, the modified BoNT polypeptide is a chimeric toxin, wherein protease domain and translocation domain comprise the BoNT/E serotype. The protease and transmembrane domain of BoNT/E comprises about amino acids 1-844 of SEQ ID NO: 100. It is to be understood that the border of the BoNT/E protease and transmembrane domain may vary by 1-10 amino acids. For example, the BoNT/E protease and transmembrane domain that may be used for the chimeric toxin may comprise amino acids 1-834, 1-835, 1-836, 1-837, 1-838, 1-839, 1-840, 1-841, 1-842, 1-843, 1-844, 1-845, 1-846, 1-847, 1-848, 1-849, 1-850, 1-851, 1-852, 1-853, 1-854 of SEQ ID NO: 100.

In some embodiments, the modified BoNT polypeptide is a chimeric toxin, wherein protease domain and translocation domain comprise the BoNT/F serotype. The protease and transmembrane domain of BoNT/F comprises about amino acids 1-863 of SEQ ID NO: 101. It is to be understood that the border of the BoNT/F protease and transmembrane domain may vary by 1-10 amino acids. For example, the BoNT/F protease and transmembrane domain that may be used for the chimeric toxin may comprise amino acids 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, 1-872, or 1-873 of SEQ ID NO: 101.

In some embodiments, the modified BoNT polypeptide is a chimeric toxin, wherein protease domain and translocation domain comprise the BoNT/G serotype. The protease and transmembrane domain of BoNT/G comprises about amino acids 1-862 of SEQ ID NO: 105. It is to be understood that the border of the BoNT/G protease and transmembrane domain may vary by 1-10 amino acids. For example, the BoNT/G protease and transmembrane domain that may be used for the chimeric toxin may comprise amino acids 1-852, 1-853, 1-854, 1-855, 1-856, 1-857, 1-858, 1-859, 1-860, 1-861, 1-862, 1-863, 1-864, 1-865, 1-866, 1-867, 1-868, 1-869, 1-870, 1-871, or 1-872 of SEQ ID NO: 105.

In some embodiments, the modified BoNT polypeptide is a chimeric toxin, wherein protease domain and translocation domain comprise the BoNT/H serotype. The protease and transmembrane domain of BoNT/H comprises about amino acids 1-858 of SEQ ID NO: 102. It is to be understood that the border of the BoNT/H protease and transmembrane domain may vary by 1-10 amino acids. For example, the BoNT/H protease and transmembrane domain that may be used for the chimeric toxin may comprise amino acids 1-848, 1-849, 1-850, 1-851, 1-852, 1-853, 1-854, 1-855, 1-856, 1-857, 1-858, 1-859, 1-860, 1-861, 1-862, 1-863, 1-864, 1-865, 1-866, 1-867 or 1-868 of SEQ ID NO: 102.

In some embodiments, the modified BoNT polypeptide is a chimeric toxin, wherein protease domain and translocation domain comprise the BoNT/X serotype as described in U.S. application Ser. No. 16/315,698 (incorporated herein by reference). The protease and transmembrane domain of BoNT/X comprises about amino acids 1-889 of SEQ ID NO: 103. It is to be understood that the border of the BoNT/X protease and transmembrane domain may vary by 1-10 amino acids. For example, the BoNT/X protease and transmembrane domain that may be used for the chimeric toxin may comprise amino acids 1-879, 1-880, 1-881, 1-882, 1-883, 1-884, 1-885, 1-886, 1-887, 1-888, 1-889, 1-890, 1-891, 1-892, 1-893, 1-894, 1-895, 1-896, 1-897, 1-898 or 1-899 of SEQ ID NO: 103.

In some embodiments, the modified BoNT polypeptide is a chimeric toxin, wherein protease domain and translocation domain comprise the BoNT/EN serotype as described in U.S. application Ser. No. 16/651,720 (incorporated herein by reference). The protease and transmembrane domain of BoNT/EN comprises about amino acids 1-874 of SEQ ID NO: 104. It is to be understood that the border of the BoNT/EN protease and transmembrane domain may vary by 1-10 amino acids. For example, the BoNT/EN protease and transmembrane domain that may be used for the chimeric toxin may comprise amino acids 1-864, 1-865, 1-866, 1-867, 1-868, 1-869, 1-870, 1-871, 1-872, 1-873, 1-874, 1-875, 1-876, 1-877, 1-878, 1-879, 1-880, 1-881, 1-882, 1-883 or 1-884 of SEQ ID NO: 104.

Some aspects of the present disclosure provide modified Clostridial Botulinum neurotoxin (BoNT) polypeptides comprising a modified receptor binding domain of Clostridial Botulinum serotype A1 (BoNT/A1). In some embodiments, the modified receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified receptor binding domain of BoNT/A1 comprises one or more amino acid substitutions at positions corresponding to 954, 955, 957, 1063, 1064, 1025, 1026, 1156, 1232, 1278, 1294, and 1295 in SEQ ID NO: 1.

In some embodiments, the modified receptor binding domain comprises an amino acid substitution at a position corresponding to 917 in SEQ ID NO: 1. In some embodiments, the modified receptor binding domain comprises an amino acid substitution corresponding to F917R or F917K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to F917R or F917K in SEQ ID NO: 49. In some embodiments, the modified BoNT polypeptide comprises the amino acid sequence of SEQ ID NO: 51 or SEQ ID NO: 52, In some embodiments, the modified BoNT polypeptide consists of the amino acid sequence of SEQ ID NO: 51 or SEQ ID NO: 52.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to F917R or F917K in SEQ ID NO: 1. In some embodiments, the modified BoNT polypeptide comprises the amino acid sequence of SEQ ID NO: 3 or SEQ ID NO: 4. In some embodiments, the modified BoNT polypeptide consists of the amino acid sequence of SEQ ID NO: 3 or SEQ ID NO: 4.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to F917R or F917K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to F917R or F917K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 51 or SEQ ID NO: 52. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 51 or SEQ ID NO: 52. In some embodiments, the modified BoNT polypeptide comprises the amino acid sequence of any one of SEQ ID NOs: 105-113 having an amino acid substitution corresponding to F917R or F917K in SEQ ID NO: 1.

In some embodiments, the modified receptor binding domain comprises an amino acid substitution at a position corresponding to 953 in SEQ ID NO: 1. In some embodiments, the modified receptor binding domain comprises an amino acid substitution corresponding to F953H or F953Y in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to F953H or F953Y in SEQ ID NO: 49. In some embodiments, the modified BoNT polypeptide comprises the amino acid sequence of SEQ ID NO: 53 or SEQ ID NO: 54, In some embodiments, the modified BoNT polypeptide consists of the amino acid sequence of SEQ ID NO: 53 or SEQ ID NO: 54.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to F953H or F953Y in SEQ ID NO: 1. In some embodiments, the modified BoNT polypeptide comprises the amino acid sequence of SEQ ID NO: 5 or SEQ ID NO: 6. In some embodiments, the modified BoNT polypeptide consists of the amino acid sequence of SEQ ID NO: 5 or SEQ ID NO: 6.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to F953H or F953Y in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to F953H or F953Y in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 53 or SEQ ID NO: 54. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 53 or SEQ ID NO: 54. In some embodiments, the modified BoNT polypeptide comprises the amino acid sequence of any one of SEQ ID NOs: 105-113 having an amino acid substitution corresponding to F953H or F953Y in SEQ ID NO: 1.

In some embodiments, the modified receptor binding domain comprises an amino acid substitution at a position corresponding to 954 in SEQ ID NO: 1. In some embodiments, the modified receptor binding domain comprises an amino acid substitution corresponding to N954S in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to N954S in SEQ ID NO: 49. In some embodiments, the modified BoNT polypeptide comprises the amino acid sequence of SEQ ID NO: 55, In some embodiments, the modified BoNT polypeptide consists of the amino acid sequence of SEQ ID NO: 55.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to N954S in SEQ ID NO: 1. In some embodiments, the modified BoNT polypeptide comprises the amino acid sequence of SEQ ID NO: 7. In some embodiments, the modified BoNT polypeptide consists of the amino acid sequence of SEQ ID NO: 7.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to N954S in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to N954S in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 55. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 55. In some embodiments, the modified BoNT polypeptide comprises the amino acid sequence of any one of SEQ ID NOs: 105-113 having an amino acid substitution corresponding to N954S in SEQ ID NO: 1.

In some embodiments, the modified receptor binding domain comprises an amino acid substitution at a position corresponding to 955 in SEQ ID NO: 1. In some embodiments, the modified receptor binding domain comprises an amino acid substitution corresponding to S955K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to S955K in SEQ ID NO: 49. In some embodiments, the modified BoNT polypeptide comprises the amino acid sequence of SEQ ID NO: 56, In some embodiments, the modified BoNT polypeptide consists of the amino acid sequence of SEQ ID NO: 56.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to S955K in SEQ ID NO: 1. In some embodiments, the modified BoNT polypeptide comprises the amino acid sequence of SEQ ID NO: 8. In some embodiments, the modified BoNT polypeptide consists of the amino acid sequence of SEQ ID NO: 8.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to S955K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to S955K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 56. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 56. In some embodiments, the modified BoNT polypeptide comprises the amino acid sequence of any one of SEQ ID NOs: 105-113 having an amino acid substitution corresponding to S955K in SEQ ID NO: 1.

In some embodiments, the modified receptor binding domain comprises an amino acid substitution at a position corresponding to 957 in SEQ ID NO: 1. In some embodiments, the modified receptor binding domain comprises an amino acid substitution corresponding to S957N in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to S957N in SEQ ID NO: 49. In some embodiments, the modified BoNT polypeptide comprises the amino acid sequence of SEQ ID NO: 57, In some embodiments, the modified BoNT polypeptide consists of the amino acid sequence of SEQ ID NO: 57.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to S957N in SEQ ID NO: 1. In some embodiments, the modified BoNT polypeptide comprises the amino acid sequence of SEQ ID NO: 9. In some embodiments, the modified BoNT polypeptide consists of the amino acid sequence of SEQ ID NO: 9.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to S957N in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to S957N in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 57. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 57. In some embodiments, the modified BoNT polypeptide comprises the amino acid sequence of any one of SEQ ID NOs: 105-113 having an amino acid substitution corresponding S957N in SEQ ID NO: 1.

In some embodiments, the modified receptor binding domain comprises an amino acid substitution at a position corresponding to 968 in SEQ ID NO: 1. In some embodiments, the modified receptor binding domain comprises an amino acid substitution corresponding to M968I in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to M968I in SEQ ID NO: 49. In some embodiments, the modified BoNT polypeptide comprises the amino acid sequence of SEQ ID NO: 58, In some embodiments, the modified BoNT polypeptide consists of the amino acid sequence of SEQ ID NO: 58.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to M968I in SEQ ID NO: 1. In some embodiments, the modified BoNT polypeptide comprises the amino acid sequence of SEQ ID NO: 10. In some embodiments, the modified BoNT polypeptide consists of the amino acid sequence of SEQ ID NO: 10.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to M968I in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to M968I in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 58. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 58. In some embodiments, the modified BoNT polypeptide comprises the amino acid sequence of any one of SEQ ID NOs: 105-113 having an amino acid substitution corresponding to M968I in SEQ ID NO: 1.

In some embodiments, the modified receptor binding domain comprises an amino acid substitution at a position corresponding to 1025 in SEQ ID NO: 1. In some embodiments, the modified receptor binding domain comprises an amino acid substitution corresponding to N1025T in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to N1025T in SEQ ID NO: 49. In some embodiments, the modified BoNT polypeptide comprises the amino acid sequence of SEQ ID NO: 59, In some embodiments, the modified BoNT polypeptide consists of the amino acid sequence of SEQ ID NO: 59.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to N1025T in SEQ ID NO: 1. In some embodiments, the modified BoNT polypeptide comprises the amino acid sequence of SEQ ID NO: 11. In some embodiments, the modified BoNT polypeptide consists of the amino acid sequence of SEQ ID NO: 11.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to N1025T in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to N1025T in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 59. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 59. In some embodiments, the modified BoNT polypeptide comprises the amino acid sequence of any one of SEQ ID NOs: 105-113 having an amino acid substitution corresponding to N1025T in SEQ ID NO: 1.

In some embodiments, the modified receptor binding domain comprises an amino acid substitution at a position corresponding to 1026 in SEQ ID NO: 1. In some embodiments, the modified receptor binding domain comprises an amino acid substitution corresponding to N1026K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to N1026K in SEQ ID NO: 49. In some embodiments, the modified BoNT polypeptide comprises the amino acid sequence of SEQ ID NO: 60, In some embodiments, the modified BoNT polypeptide consists of the amino acid sequence of SEQ ID NO: 60.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to N1026K in SEQ ID NO: 1. In some embodiments, the modified BoNT polypeptide comprises the amino acid sequence of SEQ ID NO: 12. In some embodiments, the modified BoNT polypeptide consists of the amino acid sequence of SEQ ID NO: 12.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to N1026K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to N1026K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 60. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 60. In some embodiments, the modified BoNT polypeptide comprises the amino acid sequence of any one of SEQ ID NOs: 105-113 having an amino acid substitution corresponding to N1026K in SEQ ID NO: 1.

In some embodiments, the modified receptor binding domain comprises an amino acid substitution at a position corresponding to 1052 in SEQ ID NO: 1. In some embodiments, the modified receptor binding domain comprises an amino acid substitution corresponding to N1052K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to N1052K in SEQ ID NO: 49. In some embodiments, the modified BoNT polypeptide comprises the amino acid sequence of SEQ ID NO: 61. In some embodiments, the modified BoNT polypeptide consists of the amino acid sequence of SEQ ID NO: 61.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to N1052K in SEQ ID NO: 1. In some embodiments, the modified BoNT polypeptide comprises the amino acid sequence of SEQ ID NO: 13. In some embodiments, the modified BoNT polypeptide consists of the amino acid sequence of SEQ ID NO: 13.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to N1052K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to N1052K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 61. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 61. In some embodiments, the modified BoNT polypeptide comprises the amino acid sequence of any one of SEQ ID NOs: 105-113 having an amino acid substitution corresponding to N1052K in SEQ ID NO: 1.

In some embodiments, the modified receptor binding domain comprises an amino acid substitution at a position corresponding to 1062 in SEQ ID NO: 1. In some embodiments, the modified receptor binding domain comprises an amino acid substitution corresponding to D1062E in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to D1062E in SEQ ID NO: 49. In some embodiments, the modified BoNT polypeptide comprises the amino acid sequence of SEQ ID NO: 62. In some embodiments, the modified BoNT polypeptide consists of the amino acid sequence of SEQ ID NO: 62.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to D1062E in SEQ ID NO: 1. In some embodiments, the modified BoNT polypeptide comprises the amino acid sequence of SEQ ID NO: 14. In some embodiments, the modified BoNT polypeptide consists of the amino acid sequence of SEQ ID NO: 14.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to D1062E in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to D1062E in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 62. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 62. In some embodiments, the modified BoNT polypeptide comprises the amino acid sequence of any one of SEQ ID NOs: 105-113 having an amino acid substitution corresponding to D1062E in SEQ ID NO: 1.

In some embodiments, the modified receptor binding domain comprises an amino acid substitution at a position corresponding to 1063 in SEQ ID NO: 1. In some embodiments, the modified receptor binding domain comprises an amino acid substitution corresponding to T1063P in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to T1063P in SEQ ID NO: 49. In some embodiments, the modified BoNT polypeptide comprises the amino acid sequence of SEQ ID NO: 63. In some embodiments, the modified BoNT polypeptide consists of the amino acid sequence of SEQ ID NO: 63.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to T1063P in SEQ ID NO: 1. In some embodiments, the modified BoNT polypeptide comprises the amino acid sequence of SEQ ID NO: 15. In some embodiments, the modified BoNT polypeptide consists of the amino acid sequence of SEQ ID NO: 15.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to T1063P in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to T1063P in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 63. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 63. In some embodiments, the modified BoNT polypeptide comprises the amino acid sequence of any one of SEQ ID NOs: 105-113 having an amino acid substitution corresponding to T1063P in SEQ ID NO: 1.

In some embodiments, the modified receptor binding domain comprises an amino acid substitution at a position corresponding to 1064 in SEQ ID NO: 1. In some embodiments, the modified receptor binding domain comprises an amino acid substitution corresponding to H1064R or H1064Q in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to H1064R or H1064Q in SEQ ID NO: 49. In some embodiments, the modified BoNT polypeptide comprises the amino acid sequence of SEQ ID NO: 64 or SEQ ID NO: 65. In some embodiments, the modified BoNT polypeptide consists of the amino acid sequence of SEQ ID NO: 64 or SEQ ID NO: 65.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to H1064R or H1064Q in SEQ ID NO: 1. In some embodiments, the modified BoNT polypeptide comprises the amino acid sequence of SEQ ID NO: 16 or SEQ ID NO: 17. In some embodiments, the modified BoNT polypeptide consists of the amino acid sequence of SEQ ID NO: 16 or SEQ ID NO: 17.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to H1064R or H1064Q in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to H1064R or H1064Q in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 64 or SEQ ID NO: 65. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 64 or SEQ ID NO: 65. In some embodiments, the modified BoNT polypeptide comprises the amino acid sequence of any one of SEQ ID NOs: 105-113 having an amino acid substitution corresponding to H1064R or H1064Q in SEQ ID NO: 1.

In some embodiments, the modified receptor binding domain comprises an amino acid substitution at a position corresponding to 1065 in SEQ ID NO: 1. In some embodiments, the modified receptor binding domain comprises an amino acid substitution corresponding to R1065N in SEQ ID NO: 1. These modified receptor binding domains can be referred to as BoNT/A1-R1065* (any amino acid substitution at position corresponding to 1064) and BoNT/A1-R1065N.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to R1065N in SEQ ID NO: 49. In some embodiments, the modified BoNT polypeptide comprises the amino acid sequence of SEQ ID NO: 66. In some embodiments, the modified BoNT polypeptide consists of the amino acid sequence of SEQ ID NO: 66.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to R1065N in SEQ ID NO: 1. In some embodiments, the modified BoNT polypeptide comprises the amino acid sequence of SEQ ID NO: 18. In some embodiments, the modified BoNT polypeptide consists of the amino acid sequence of SEQ ID NO: 18.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to R1065N in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to R1065N in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 66. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 66. In some embodiments, the modified BoNT polypeptide comprises the amino acid sequence of any one of SEQ ID NOs: 105-113 having an amino acid substitution corresponding to R1065N in SEQ ID NO: 1.

In some embodiments, the modified receptor binding domain comprises an amino acid substitution at a position corresponding to 1066 in SEQ ID NO: 1. In some embodiments, the modified receptor binding domain comprises an amino acid substitution corresponding to Y1066R or Y1066K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to Y1066R or Y1066K in SEQ ID NO: 49. In some embodiments, the modified BoNT polypeptide comprises the amino acid sequence of SEQ ID NO: 67 or SEQ ID NO: 68. In some embodiments, the modified BoNT polypeptide consists of the amino acid sequence of SEQ ID NO: 67 or SEQ ID NO: 68.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to Y1066R or Y1066K in SEQ ID NO: 1. In some embodiments, the modified BoNT polypeptide comprises the amino acid sequence of SEQ ID NO: 19 or SEQ ID NO: 20. In some embodiments, the modified BoNT polypeptide consists of the amino acid sequence of SEQ ID NO: 19 or SEQ ID NO: 20.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to Y1066R or Y1066K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to Y1066R or Y1066K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 67 or SEQ ID NO: 68. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 67 or SEQ ID NO: 68. In some embodiments, the modified BoNT polypeptide comprises the amino acid sequence of any one of SEQ ID NOs: 105-113 having an amino acid substitution corresponding to Y1066R or Y1066K in SEQ ID NO: 1.

In some embodiments, the modified receptor binding domain comprises an amino acid substitution at a position corresponding to 1145 in SEQ ID NO: 1. In some embodiments, the modified receptor binding domain comprises an amino acid substitution corresponding to T1145Y in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to T1145Y in SEQ ID NO: 49. In some embodiments, the modified BoNT polypeptide comprises the amino acid sequence of SEQ ID NO: 69. In some embodiments, the modified BoNT polypeptide consists of the amino acid sequence of SEQ ID NO: 69.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to T1145Y in SEQ ID NO: 1. In some embodiments, the modified BoNT polypeptide comprises the amino acid sequence of SEQ ID NO: 21. In some embodiments, the modified BoNT polypeptide consists of the amino acid sequence of SEQ ID NO: 21.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to T1145Y in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to T1145Y in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 69. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 69. In some embodiments, the modified BoNT polypeptide comprises the amino acid sequence of any one of SEQ ID NOs: 105-113 having an amino acid substitution corresponding to T1145Y in SEQ ID NO: 1.

In some embodiments, the modified receptor binding domain comprises an amino acid substitution at a position corresponding to 1156 in SEQ ID NO: 1. In some embodiments, the modified receptor binding domain comprises an amino acid substitution corresponding to R1156M or R1156I in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to R1156M or R1156I in SEQ ID NO: 49. In some embodiments, the modified BoNT polypeptide comprises the amino acid sequence of SEQ ID NO: 70 or SEQ ID NO: 71. In some embodiments, the modified BoNT polypeptide consists of the amino acid sequence of SEQ ID NO: 70 or SEQ ID NO: 71.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to R1156M or R1156I in SEQ ID NO: 1. In some embodiments, the modified BoNT polypeptide comprises the amino acid sequence of SEQ ID NO: 22 or SEQ ID NO: 23. In some embodiments, the modified BoNT polypeptide consists of the amino acid sequence of SEQ ID NO: 22 or SEQ ID NO: 23.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to R1156M or R1156I in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to R1156M or R1156I in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 22 or SEQ ID NO: 23. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 22 or SEQ ID NO: 23. In some embodiments, the modified BoNT polypeptide comprises the amino acid sequence of any one of SEQ ID NOs: 105-113 having an amino acid substitution corresponding to R1156M or R1156I in SEQ ID NO: 1.

In some embodiments, the modified receptor binding domain comprises an amino acid substitution at a position corresponding to 1232 in SEQ ID NO: 1. In some embodiments, the modified receptor binding domain comprises an amino acid substitution corresponding to T1232R or T1232K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to T1232R or T1232K in SEQ ID NO: 49. In some embodiments, the modified BoNT polypeptide comprises the amino acid sequence of SEQ ID NO: 72 or SEQ ID NO: 73. In some embodiments, the modified BoNT polypeptide consists of the amino acid sequence of SEQ ID NO: 72 or SEQ ID NO: 73.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to T1232R or T1232K in SEQ ID NO: 1. In some embodiments, the modified BoNT polypeptide comprises the amino acid sequence of SEQ ID NO: 24 or SEQ ID NO: 25. In some embodiments, the modified BoNT polypeptide consists of the amino acid sequence of SEQ ID NO: 24 or SEQ ID NO: 25.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to T1232R or T1232K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to T1232R or T1232K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 72 or SEQ ID NO: 73. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 72 or SEQ ID NO: 73. In some embodiments, the modified BoNT polypeptide comprises the amino acid sequence of any one of SEQ ID NOs: 105-113 having an amino acid substitution corresponding to T1232R or T1232K in SEQ ID NO: 1.

In some embodiments, the modified receptor binding domain comprises an amino acid substitution at a position corresponding to 1272 in SEQ ID NO: 1. In some embodiments, the modified receptor binding domain comprises an amino acid substitution corresponding to E1272G in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to E1272G in SEQ ID NO: 49. In some embodiments, the modified BoNT polypeptide comprises the amino acid sequence of SEQ ID NO: 74. In some embodiments, the modified BoNT polypeptide consists of the amino acid sequence of SEQ ID NO: 74.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to E1272G in SEQ ID NO: 1. In some embodiments, the modified BoNT polypeptide comprises the amino acid sequence of SEQ ID NO: 26. In some embodiments, the modified BoNT polypeptide consists of the amino acid sequence of SEQ ID NO: 26.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to E1272G in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to E1272G in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 74. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 74. In some embodiments, the modified BoNT polypeptide comprises the amino acid sequence of any one of SEQ ID NOs: 105-113 having an amino acid substitution corresponding to E1272G in SEQ ID NO: 1.

In some embodiments, the modified receptor binding domain comprises an amino acid substitution at a position corresponding to 1278 in SEQ ID NO: 1. In some embodiments, the modified receptor binding domain comprises an amino acid substitution corresponding to L1278F, L1278Y or L1278W in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to L1278F, L1278Y or L1278W in SEQ ID NO: 49. In some embodiments, the modified BoNT polypeptide comprises the amino acid sequence of SEQ ID NO: 75, SEQ ID NO: 76 or SEQ ID NO: 77. In some embodiments, the modified BoNT polypeptide consists of the amino acid sequence of SEQ ID NO: 75, SEQ ID NO: 76 or SEQ ID NO: 77.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to L1278F, L1278Y or L1278W in SEQ ID NO: 1. In some embodiments, the modified BoNT polypeptide comprises the amino acid sequence of SEQ ID NO: 27, SEQ ID NO: 28 or SEQ ID NO: 29. In some embodiments, the modified BoNT polypeptide consists of the amino acid sequence of SEQ ID NO: 27, SEQ ID NO: 28 or SEQ ID NO: 29.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to L1278F, L1278Y or L1278W in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to L1278F, L1278Y or L1278W in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 75, SEQ ID NO: 76 or SEQ ID NO: 77. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 75, SEQ ID NO: 76 or SEQ ID NO: 77. In some embodiments, the modified BoNT polypeptide comprises the amino acid sequence of any one of SEQ ID NOs: 105-113 having an amino acid substitution corresponding to L1278F, L1278Y or L1278W in SEQ ID NO: 1.

In some embodiments, the modified receptor binding domain comprises an amino acid substitution at a position corresponding to 1288 in SEQ ID NO: 1. In some embodiments, the modified receptor binding domain comprises an amino acid substitution corresponding to D1288E in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to D1288E in SEQ ID NO: 49. In some embodiments, the modified BoNT polypeptide comprises the amino acid sequence of SEQ ID NO: 78. In some embodiments, the modified BoNT polypeptide consists of the amino acid sequence of SEQ ID NO: 78.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to D1288E in SEQ ID NO: 1. In some embodiments, the modified BoNT polypeptide comprises the amino acid sequence of SEQ ID NO: 30. In some embodiments, the modified BoNT polypeptide consists of the amino acid sequence of SEQ ID NO: 30.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to D1288E in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to D1288E in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 78. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 78. In some embodiments, the modified BoNT polypeptide comprises the amino acid sequence of any one of SEQ ID NOs: 105-113 having an amino acid substitution corresponding to D1288E in SEQ ID NO: 1.

In some embodiments, the modified receptor binding domain comprises an amino acid substitution at a position corresponding to 1289 in SEQ ID NO: 1. In some embodiments, the modified receptor binding domain comprises an amino acid substitution corresponding to D1289Y in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to D1289Y in SEQ ID NO: 49. In some embodiments, the modified BoNT polypeptide comprises the amino acid sequence of SEQ ID NO: 79. In some embodiments, the modified BoNT polypeptide consists of the amino acid sequence of SEQ ID NO: 79.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to D1289Y in SEQ ID NO: 1. In some embodiments, the modified BoNT polypeptide comprises the amino acid sequence of SEQ ID NO: 31. In some embodiments, the modified BoNT polypeptide consists of the amino acid sequence of SEQ ID NO: 31.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to D1289Y in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to D1289Y in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 79. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 79. In some embodiments, the modified BoNT polypeptide comprises the amino acid sequence of any one of SEQ ID NOs: 105-113 having an amino acid substitution corresponding to D1289Y in SEQ ID NO: 1.

In some embodiments, the modified receptor binding domain comprises an amino acid substitution at a position corresponding to 1292 in SEQ ID NO: 1. In some embodiments, the modified receptor binding domain comprises an amino acid substitution corresponding to G1292R or G1292K in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to G1292R or G1292K in SEQ ID NO: 49. In some embodiments, the modified BoNT polypeptide comprises the amino acid sequence of SEQ ID NO: 80 or SEQ ID NO: 81. In some embodiments, the modified BoNT polypeptide consists of the amino acid sequence of SEQ ID NO: 80 or SEQ ID NO: 81.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to G1292R or G1292K in SEQ ID NO: 1. In some embodiments, the modified BoNT polypeptide comprises the amino acid sequence of SEQ ID NO: 32 or SEQ ID NO: 33. In some embodiments, the modified BoNT polypeptide consists of the amino acid sequence of SEQ ID NO: 32 or SEQ ID NO: 33.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to G1292R or G1292K in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to G1292R or G1292K in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 80 or SEQ ID NO: 81. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 80 or SEQ ID NO: 81. In some embodiments, the modified BoNT polypeptide comprises the amino acid sequence of any one of SEQ ID NOs: 105-113 having an amino acid substitution corresponding to G1292R or G1292K in SEQ ID NO: 1.

In some embodiments, the modified receptor binding domain comprises an amino acid substitution at a position corresponding to R1294S in SEQ ID NO: 1. In some embodiments, the modified receptor binding domain comprises an amino acid substitution corresponding to R1294S or R1294T in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to R1294S or R1294T in SEQ ID NO: 49. In some embodiments, the modified BoNT polypeptide comprises the amino acid sequence of SEQ ID NO: 82 or SEQ ID NO: 83. In some embodiments, the modified BoNT polypeptide consists of the amino acid sequence of SEQ ID NO: 82 or SEQ ID NO: 83.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to R1294S or R1294T in SEQ ID NO: 1. In some embodiments, the modified BoNT polypeptide comprises the amino acid sequence of SEQ ID NO: 34 or SEQ ID NO: 35. In some embodiments, the modified BoNT polypeptide consists of the amino acid sequence of SEQ ID NO: 34 or SEQ ID NO: 35.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to R1294S or R1294T in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to R1294S or R1294T in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 82 or SEQ ID NO: 83. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 82 or SEQ ID NO: 83. In some embodiments, the modified BoNT polypeptide comprises the amino acid sequence of any one of SEQ ID NOs: 105-113 having an amino acid substitution corresponding to R1294S or R1294T in SEQ ID NO: 1.

In some embodiments, the modified receptor binding domain comprises an amino acid substitution at a position corresponding to 1295 in SEQ ID NO: 1. In some embodiments, the modified receptor binding domain comprises an amino acid substitution corresponding to P1295S or P1295T in SEQ ID NO: 1.

In some embodiments, the modified BoNT polypeptide is a modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 49, and comprises an amino acid substitution corresponding to P1295S or P1295T in SEQ ID NO: 49. In some embodiments, the modified BoNT polypeptide comprises the amino acid sequence of SEQ ID NO: 84 or SEQ ID NO: 85. In some embodiments, the modified BoNT polypeptide consists of the amino acid sequence of SEQ ID NO: 84 or SEQ ID NO: 85.

In some embodiments, the modified BoNT polypeptide is a full-length BoNT/A1 polypeptide comprising the modified receptor binding domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an amino acid substitution corresponding to P1295S or P1295T in SEQ ID NO: 1. In some embodiments, the modified BoNT polypeptide comprises the amino acid sequence of SEQ ID NO: 36 or SEQ ID NO: 37. In some embodiments, the modified BoNT polypeptide consists of the amino acid sequence of SEQ ID NO: 36 or SEQ ID NO: 37.

In some embodiments, the modified BoNT polypeptide is a chimeric BoNT polypeptide comprising the modified receptor binding of BoNT/A1 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid substitution corresponding to P1295S or P1295T in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A1 comprising an amino acid sequence that is at least 70% (e.g., at least 70%, at least 75%, at least 80%, at least 85%, at least 90%, at least 95%, or at least 99%) identical to SEQ ID NO: 49 and comprises an amino acid substitution corresponding to P1295S or P1295T in SEQ ID NO: 1. In some embodiments, the modified BoNT polypeptide comprises a polypeptide comprising the amino acid sequence of any one of SEQ ID NOs: 97-105, fused to a modified receptor bonding domain of BoNT/A1 comprising the amino acid sequence of SEQ ID NO: 84 or SEQ ID NO: 85. In some embodiments, the modified BoNT polypeptide comprises a polypeptide consisting of the amino acid sequence of any one of SEQ ID NOs: 97-105, fused to a modified receptor bonding domain of BoNT/A1 consisting of the amino acid sequence of SEQ ID NO: 84 or SEQ ID NO: 85. In some embodiments, the modified BoNT polypeptide comprises the amino acid sequence of any one of SEQ ID NOs: 105-113 having an amino acid substitution corresponding to P1295S or P1295T in SEQ ID NO: 1.

Some aspects of the present disclosure provide modified Clostridial Botulinum neurotoxin (BoNT) polypeptides comprising a modified receptor binding domain of Clostridial Botulinum serotype A2 (BoNT/A2). In some embodiments, the modified receptor binding domain of BoNT/A2 comprises one or more amino acid substitutions at positions corresponding to 915, 923, 1090, 1103, 1117, 1156, 1170, 1227, 1254, 1255, or 1256 in SEQ ID NO: 2. in SEQ ID NO: 2.

In some embodiments, the modified receptor binding domain comprises an amino acid substitution at a position corresponding to 915 in SEQ ID NO: 2. In some embodiments, the modified receptor binding domain comprises an amino acid substitution corresponding to K915Q in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to K915Q in SEQ ID NO: 50. In some embodiments, the modified BoNT polypeptide comprises the amino acid sequence of SEQ ID NO: 86. In some embodiments, the modified BoNT polypeptide consists of the amino acid sequence of SEQ ID NO: 86.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to K915Q in SEQ ID NO: 2. In some embodiments, the modified BoNT polypeptide comprises the amino acid sequence of SEQ ID NO: 38. In some embodiments, the modified BoNT polypeptide consists of the amino acid sequence of SEQ ID NO: 38.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to K915Q in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to K915Q in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 86. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 86. In some embodiments, the modified BoNT polypeptide comprises the amino acid sequence of any one of SEQ ID NOs: 114-122 having an amino acid substitution corresponding to K915Q in SEQ ID NO: 2.

In some embodiments, the modified receptor binding domain comprises an amino acid substitution at a position corresponding to 923 in SEQ ID NO: 2. In some embodiments, the modified receptor binding domain comprises an amino acid substitution corresponding to T923K in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to T923K in SEQ ID NO: 50. In some embodiments, the modified BoNT polypeptide comprises the amino acid sequence of SEQ ID NO: 87. In some embodiments, the modified BoNT polypeptide consists of the amino acid sequence of SEQ ID NO: 87.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to T923K in SEQ ID NO: 2. In some embodiments, the modified BoNT polypeptide comprises the amino acid sequence of SEQ ID NO: 39. In some embodiments, the modified BoNT polypeptide consists of the amino acid sequence of SEQ ID NO: 39.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to T923K in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to T923K in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 87. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 87. In some embodiments, the modified BoNT polypeptide comprises the amino acid sequence of any one of SEQ ID NOs: 114-122 having an amino acid substitution corresponding to T923K in SEQ ID NO: 2.

In some embodiments, the modified receptor binding domain comprises an amino acid substitution at a position corresponding to 1090 in SEQ ID NO: 2. In some embodiments, the modified receptor binding domain comprises an amino acid substitution corresponding to S1090N in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to S1090N in SEQ ID NO: 50. In some embodiments, the modified BoNT polypeptide comprises the amino acid sequence of SEQ ID NO: 88. In some embodiments, the modified BoNT polypeptide consists of the amino acid sequence of SEQ ID NO: 88.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to S1090N in SEQ ID NO: 2. In some embodiments, the modified BoNT polypeptide comprises the amino acid sequence of SEQ ID NO: 40. In some embodiments, the modified BoNT polypeptide consists of the amino acid sequence of SEQ ID NO: 40.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to S1090N in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to S1090N in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 88. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 88. In some embodiments, the modified BoNT polypeptide comprises the amino acid sequence of any one of SEQ ID NOs: 114-122 having an amino acid substitution corresponding to S1090N in SEQ ID NO: 2.

In some embodiments, the modified receptor binding domain comprises an amino acid substitution at a position corresponding to 1103 in SEQ ID NO: 2. In some embodiments, the modified receptor binding domain comprises an amino acid substitution corresponding to N1103D in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to N1103D in SEQ ID NO: 50. In some embodiments, the modified BoNT polypeptide comprises the amino acid sequence of SEQ ID NO: 89. In some embodiments, the modified BoNT polypeptide consists of the amino acid sequence of SEQ ID NO: 89.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to N1103D in SEQ ID NO: 2. In some embodiments, the modified BoNT polypeptide comprises the amino acid sequence of SEQ ID NO: 41. In some embodiments, the modified BoNT polypeptide consists of the amino acid sequence of SEQ ID NO: 41.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to N1103D in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to N1103D in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 89. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 89. In some embodiments, the modified BoNT polypeptide comprises the amino acid sequence of any one of SEQ ID NOs: 114-122 having an amino acid substitution corresponding to N1103D in SEQ ID NO: 2.

In some embodiments, the modified receptor binding domain comprises an amino acid substitution at a position corresponding to 1117 in SEQ ID NO: 2. In some embodiments, the modified receptor binding domain comprises an amino acid substitution corresponding to F1117Y in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to F1117Y in SEQ ID NO: 50. In some embodiments, the modified BoNT polypeptide comprises the amino acid sequence of SEQ ID NO: 90. In some embodiments, the modified BoNT polypeptide consists of the amino acid sequence of SEQ ID NO: 90.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to F1117Y in SEQ ID NO: 2. In some embodiments, the modified BoNT polypeptide comprises the amino acid sequence of SEQ ID NO: 42. In some embodiments, the modified BoNT polypeptide consists of the amino acid sequence of SEQ ID NO: 42.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to F1117Y in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to F1117Y in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 90. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 90. In some embodiments, the modified BoNT polypeptide comprises the amino acid sequence of any one of SEQ ID NOs: 114-122 having an amino acid substitution corresponding to F1117Y in SEQ ID NO: 2.

In some embodiments, the modified receptor binding domain comprises an amino acid substitution at a position corresponding to 1156 in SEQ ID NO: 2. In some embodiments, the modified receptor binding domain comprises an amino acid substitution corresponding to E1156M in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to E1156M in SEQ ID NO: 50. In some embodiments, the modified BoNT polypeptide comprises the amino acid sequence of SEQ ID NO: 91. In some embodiments, the modified BoNT polypeptide consists of the amino acid sequence of SEQ ID NO: 91.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to E1156M in SEQ ID NO: 2. In some embodiments, the modified BoNT polypeptide comprises the amino acid sequence of SEQ ID NO: 43. In some embodiments, the modified BoNT polypeptide consists of the amino acid sequence of SEQ ID NO: 43.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to E1156M in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to E1156M in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 91. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 91. In some embodiments, the modified BoNT polypeptide comprises the amino acid sequence of any one of SEQ ID NOs: 114-122 having an amino acid substitution corresponding to E1156M in SEQ ID NO: 2.

In some embodiments, the modified receptor binding domain comprises an amino acid substitution at a position corresponding to 1170 in SEQ ID NO: 2. In some embodiments, the modified receptor binding domain comprises an amino acid substitution corresponding to E1170K in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to E1170K in SEQ ID NO: 50. In some embodiments, the modified BoNT polypeptide comprises the amino acid sequence of SEQ ID NO: 92. In some embodiments, the modified BoNT polypeptide consists of the amino acid sequence of SEQ ID NO: 92.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to E1170K in SEQ ID NO: 2. In some embodiments, the modified BoNT polypeptide comprises the amino acid sequence of SEQ ID NO: 44. In some embodiments, the modified BoNT polypeptide consists of the amino acid sequence of SEQ ID NO: 44.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to E1170K in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to E1170K in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 92. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 92. In some embodiments, the modified BoNT polypeptide comprises the amino acid sequence of any one of SEQ ID NOs: 114-122 having an amino acid substitution corresponding to E1170K in SEQ ID NO: 2.

In some embodiments, the modified receptor binding domain comprises an amino acid substitution at a position corresponding to 1227 in SEQ ID NO: 2. In some embodiments, the modified receptor binding domain comprises an amino acid substitution corresponding to D1227N in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to D1227N in SEQ ID NO: 50. In some embodiments, the modified BoNT polypeptide comprises the amino acid sequence of SEQ ID NO: 93. In some embodiments, the modified BoNT polypeptide consists of the amino acid sequence of SEQ ID NO: 93.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to D1227N in SEQ ID NO: 2. In some embodiments, the modified BoNT polypeptide comprises the amino acid sequence of SEQ ID NO: 45. In some embodiments, the modified BoNT polypeptide consists of the amino acid sequence of SEQ ID NO: 45.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to D1227N in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to D1227N in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 93. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 93. In some embodiments, the modified BoNT polypeptide comprises the amino acid sequence of any one of SEQ ID NOs: 114-122 having an amino acid substitution corresponding to D1227N in SEQ ID NO: 2.

In some embodiments, the modified receptor binding domain comprises an amino acid substitution at a position corresponding to 1254 in SEQ ID NO: 2. In some embodiments, the modified receptor binding domain comprises an amino acid substitution corresponding to L1254Q in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to L1254Q in SEQ ID NO: 50. In some embodiments, the modified BoNT polypeptide comprises the amino acid sequence of SEQ ID NO: 94. In some embodiments, the modified BoNT polypeptide consists of the amino acid sequence of SEQ ID NO: 94.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to L1254Q in SEQ ID NO: 2. In some embodiments, the modified BoNT polypeptide comprises the amino acid sequence of SEQ ID NO: 46. In some embodiments, the modified BoNT polypeptide consists of the amino acid sequence of SEQ ID NO: 46.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to L1254Q in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to L1254Q in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 94. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 94. In some embodiments, the modified BoNT polypeptide comprises the amino acid sequence of any one of SEQ ID NOs: 114-122 having an amino acid substitution corresponding to L1254Q in SEQ ID NO: 2.

In some embodiments, the modified receptor binding domain comprises an amino acid substitution at a position corresponding to 1255 in SEQ ID NO: 2. In some embodiments, the modified receptor binding domain comprises an amino acid substitution corresponding to Y1255F in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to Y1255F in SEQ ID NO: 50. In some embodiments, the modified BoNT polypeptide comprises the amino acid sequence of SEQ ID NO: 95. In some embodiments, the modified BoNT polypeptide consists of the amino acid sequence of SEQ ID NO: 95.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to Y1255F in SEQ ID NO: 2. In some embodiments, the modified BoNT polypeptide comprises the amino acid sequence of SEQ ID NO: 47. In some embodiments, the modified BoNT polypeptide consists of the amino acid sequence of SEQ ID NO: 47.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to Y1255F in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to Y1255F in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 95. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 95. In some embodiments, the modified BoNT polypeptide comprises the amino acid sequence of any one of SEQ ID NOs: 114-122 having an amino acid substitution corresponding to Y1255F in SEQ ID NO: 2.

In some embodiments, the modified receptor binding domain comprises an amino acid substitution at a position corresponding to 1256 in SEQ ID NO: 2. In some embodiments, the modified receptor binding domain comprises an amino acid substitution corresponding to D1256N in SEQ ID NO: 2.

In some embodiments, the modified BoNT polypeptide is a modified receptor binding domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 50, and comprises an amino acid substitution corresponding to D1256N in SEQ ID NO: 50. In some embodiments, the modified BoNT polypeptide comprises the amino acid sequence of SEQ ID NO: 96. In some embodiments, the modified BoNT polypeptide consists of the amino acid sequence of SEQ ID NO: 96.

In some embodiments, the modified BoNT polypeptide is a full-length BoNT/A2 polypeptide comprising the modified receptor binding of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 2, and comprises an amino acid substitution corresponding to D1256N in SEQ ID NO: 2. In some embodiments, the modified BoNT polypeptide comprises the amino acid sequence of SEQ ID NO: 48. In some embodiments, the modified BoNT polypeptide consists of the amino acid sequence of SEQ ID NO: 48.

In some embodiments, the modified BoNT polypeptide is a chimeric BoNT polypeptide comprising the modified receptor binding of BoNT/A2 described herein, and a protease domain and translocation domain from a BoNT of a different serotype (e.g., BoNT/B, C, D, E, F, G, H, X, or EN). In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid substitution corresponding to D1256N in SEQ ID NO: 2. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 97-105, fused to a modified receptor bonding domain of BoNT/A2 comprising an amino acid sequence that is at least 70% (e.g., at least 70%, at least 75%, at least 80%, at least 85%, at least 90%, at least 95%, or at least 99%) identical to SEQ ID NO: 50 and comprises an amino acid substitution corresponding to D1256N in SEQ ID NO: 2. In some embodiments, the modified BoNT polypeptide comprises a polypeptide comprising the amino acid sequence of any one of SEQ ID NOs: 97-105, fused to a modified receptor bonding domain of BoNT/A2 comprising the amino acid sequence of SEQ ID NO: 96. In some embodiments, the modified BoNT polypeptide comprises a polypeptide consisting of the amino acid sequence of any one of SEQ ID NOs: 97-105, fused to a modified receptor bonding domain of BoNT/A2 consisting of the amino acid sequence of SEQ ID NO: 96. In some embodiments, the modified BoNT polypeptide comprises the amino acid sequence of any one of SEQ ID NOs: 114-122 having an amino acid substitution corresponding to D1256N in SEQ ID NO: 2.

In some embodiments, the modified BoNT receptor binding domains described herein may be referred to using a generic nomenclature as follows: H_{C}-[BoNT serotype]-[wildtype amino acid corresponding to modification] [modification position in full length BoNT serotype amino acid sequence][substitution mutation]. In a non-limiting example, H_{C}-BoNT/A1-F917* refers to a modified BoNT/A1 receptor binding domain comprising an amino acid substitution at a position corresponding to 917 in SEQ ID NO: 1, where * indicates a substitution mutation to any other amino acid. In a non-limiting example, H_{C}-BoNT/A1-F917R refers to a modified BoNT/A1 receptor binding domain comprising an amino acid substitution at a position corresponding to 917 in SEQ ID NO: 1, wherein the amino acid substitution corresponds to F917R in SEQ ID NO: 1. This nomenclature is used in Table 8.

In some embodiments, the modified BoNT described herein may be referred to using a generic nomenclature as follows: [BoNT serotype]-[wildtype amino acid corresponding to modification] [modification position in full length BoNT serotype amino acid sequence][substitution mutation]. In a non-limiting example, BoNT/A1-F917* refers to a modified BoNT/A1 comprising an amino acid substitution at a position corresponding to 917 in SEQ ID NO: 1 where * indicates a substitution mutation to any other amino acid. In a non-limiting example, BoNT/A1-F917R refers to a modified BoNT/A1 comprising an amino acid substitution at a position corresponding to 917 in SEQ ID NO: 1, wherein the amino acid substitution corresponds to F917R in SEQ ID NO: 1. This nomenclature is used in Table 8.

In some embodiments, a generic nomenclature may be used to describe a chimeric BoNT polypeptide with a modified receptor binding domain as follows: [BoNT protease and translocation domain serotype]|[BoNT receptor binding domain serotype]-[wildtype amino acid corresponding to modification] [modification position in full length BoNT serotype amino acid sequence][substitution mutation]. In a non-limiting example, BoNT/BIA1-F917* refers to the protease and transmembrane domain of BoNT/B fused with a BoNT/A1 modified receptor binding domain comprising a mutation at position 917 of SEQ ID NO: 1, where * indicates a substitution mutation to any other amino acid. In a non-limiting example, BoNT/BIA1-F917R refers to the protease and transmembrane domain of BoNT/B fused with a BoNT/A1 modified receptor binding domain comprising a phenylalanine to arginine mutation at position 917 of SEQ ID NO: 1. This nomenclature is used in Table 8.

The modified BoNT polypeptides of the present disclosure (e.g., without limitation, polypeptides comprising amino acid sequence of any of SEQ ID NOs: 3-48 and 51-96), will generally be produced by expression form recombinant nucleic acids in appropriate cells (e.g., E. coli, or insect cells) and isolated. The nucleic acids encoding the polypeptides described herein may be obtained, and the nucleotide sequence of the nucleic acids determined, by any method known in the art.

Further provided herein are isolated and/or recombinant nucleic acids encoding any of the modified BoNT polypeptides disclosed herein. The nucleic acids encoding the isolated polypeptide fragments of the present disclosure, may be DNA or RNA, double-stranded or single stranded. In certain aspects, the subject nucleic acids encoding the isolated polypeptide fragments are further understood to include nucleic acids encoding polypeptides that are variants of any of the modified BoNT polypeptides described herein.

Variant nucleotide sequences include sequences that differ by one or more nucleotide substitutions, additions or deletions, such as allelic variants. In some embodiments, the isolated nucleic acid molecule of the present disclosure comprising a polynucleotide encoding a polypeptide comprising an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identity of any of SEQ ID NOs: 3-48 and 51-96. In some embodiments, the isolated nucleic acid molecule of the present disclosure comprising a polynucleotide encoding a polypeptide comprising an amino acid sequence that has 85%, 86%, 87%, 88%, 89%, 90%, 91%, 92%, 93%, 94%, 95%, 96%, 97%, 98%, 99%, or 100% identity of any of SEQ ID NOs: 3-48 and 51-96.

In some embodiments, the nucleic acid is comprised within a vector, such as an expression vector. In some embodiments, the vector comprises a promoter operably linked to the nucleic acid.

A variety of promoters can be used for expression of the polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)].

Other systems include FK506 dimer, VP16 or p65 using astradiol, RU486, diphenol murislerone, or rapamycin. Inducible systems are available from Invitrogen, Clontech and Ariad. Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

The host cells used to express the isolated polypeptides described herein may be either bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667). A variety of host-expression vector systems may be utilized to express the isolated polypeptides described herein. Such host-expression systems represent vehicles by which the coding sequences of the isolate d polypeptides described herein may be produced and subsequently purified, but also represent cells which may, when transformed or transfected with the appropriate nucleotide coding sequences, express the isolated polypeptides described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the isolated polypeptides described herein; yeast (e.g., Saccharomyces pichia) transformed with recombinant yeast expression vectors containing sequences encoding the isolated polypeptides described herein; insect cell systems infected with recombinant virus expression vectors (e.g., baclovirus) containing the sequences encoding the isolated polypeptides described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the isolated polypeptides described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the polypeptides being expressed. For example, when a large quantity of such a protein is to be produced, for the generation of pharmaceutical compositions of polypeptides described herein, vectors which direct the expression of high levels of fusion protein products that are readily purified may be desirable. Such vectors include, but are not limited, to the E. coli expression vector pUR278 (Rüther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The 1pp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione.

The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic.

The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the polypeptides described herein may be expressed as a single gene product (e.g., as a single polypeptide chain, i.e., as a polyprotein precursor), requiring proteolytic cleavage by native or recombinant cellular mechanisms to form separate polypeptides described herein.

The disclosure thus encompasses engineering a nucleic acid sequence to encode a polyprotein precursor molecule comprising the polypeptides described herein, which includes coding sequences capable of directing post translational cleavage of said polyprotein precursor. Post-translational cleavage of the polyprotein precursor results in the polypeptides described herein. The post translational cleavage of the precursor molecule comprising the polypeptides described herein may occur in vivo (i.e., within the host cell by native or recombinant cell systems/mechanisms, e.g. furin cleavage at an appropriate site) or may occur in vitro (e.g. incubation of said polypeptide chain in a composition comprising proteases or peptidases of known activity and/or in a composition comprising conditions or reagents known to foster the desired proteolytic action).

Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express polypeptides described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptides described herein. Such engineered cell lines may be particularly useful in screening and evaluation of polypeptides that interact directly or indirectly with the polypeptides described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of polypeptides described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a polypeptide described herein is amplifiable, increase in the level of inhibitor present in culture of host cell will increase the number of copies of the marker gene. Since the amplified region is associated with the nucleotide sequence of a polypeptide described herein or a polypeptide described herein, production of the polypeptide will also increase (Crouse et al. (1983) “Expression And Amplification Of Engineered Mouse Dihydrofolate Reductase Minigenes,” Mol. Cell. Biol. 3:257-266).

Once a polypeptide described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard technique for the purification of polypeptides or antibodies. Other aspects of the present disclosure relate to a cell comprising a nucleic acid described herein or a vector described herein.

The cell may be a prokaryotic or eukaryotic cell. In some embodiments, the cell in a mammalian cell. Exemplary cell types are described herein. Other aspects of the present disclosure related to a cell expressing the modified BoNT polypeptides described herein. The cell may be a prokaryotic or eukaryotic cell. In some embodiments, the cell in a mammalian cell. Exemplary cell types are described herein. The cell can be for propagation of the nucleic acid or for expression of the nucleic acid, or both. Such cells include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4.sup.th ed. 2000); ANIMAL CELL CULTURE: A PRACTICAL APPROACH (John R. W. Masters ed., Oxford University Press, 3.sup.rd ed. 2000); MOLECULAR CLONING A LABORATORY MANUAL, supra, (2001); BASIC CELL CULTURE: A PRACTICAL APPROACH (John M. Davis, Oxford Press, 2.sup.nd ed. 2002); and CURRENT PROTOCOLS IN MOLECULAR BIOLOGY, supra, (2004).

These protocols are routine procedures within the scope of one skilled in the art and from the teaching herein. Yet other aspects of the present disclosure relate to a method of producing a polypeptide described herein, the method comprising obtaining a cell described herein and expressing nucleic acid described herein in said cell. In some embodiments, the method further comprises isolating and purifying a polypeptide described herein.

In some embodiments, botulinum neurotoxin can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive.

The bacterial strains that make botulinum toxin serotypes A and G possess endogenous proteases and serotypes A and G can therefore be recovered from bacterial cultures in predominantly their active form. In contrast, botulinum toxin serotypes Ci, D and E are synthesized by non-proteolytic strains and are therefore typically unactivated when recovered from culture. Serotypes B and F are produced by both proteolytic and non-proteolytic strains and therefore can be recovered in either the active or inactive form. The proteolytic strains that produce, for example, the botulinum toxin type A serotype may only cleave a portion of the toxin produced.

The exact proportion of nicked to un-nicked molecules depends on the length of incubation and the temperature of the culture. Therefore, a certain percentage of a preparation of, for example, the botulinum toxin type A toxin may be inactive. In one embodiment, the neurotoxin of the present disclosure is in an active state. In one embodiment, the neurotoxin is in an inactive state. In one embodiment, a combination of active and inactive neurotoxin is envisioned.

It is also envisioned that the modified receptor binding domain of BoNT/A1 or BoNT/A2 described here can be utilized as a delivery tool to target neurons in humans. For example, the modified receptor binding domain of BoNT/A1 or BoNT/A2 can be linked to other therapeutic agents, covalently or non-covalently, and acts as the targeting vehicle to deliver the therapeutic agents to neurons in humans.

As such, another aspect of the disclosure relates to a chimeric polypeptide molecule comprising a first portion that is a modified receptor binding domain of C. Botulinum serotype B, comprising one or more substitution mutation(s) which leads to significantly enhanced binding to neurons, linked to a second portion. The second portion of the molecule can be a bioactive molecule such as a therapeutic agent (e.g., a polypeptide or drug). Linkage of the first and second portions of the molecule can be covalent (e.g., in the form of a fusion protein) or non-covalent. Methods of such linkage are known in the art and can readily be applied by the skilled practitioner. When the second portion of the chimeric molecule is a polypeptide and the chimeric molecule is in the form of a protein, nucleic acids and nucleic acid vectors encoding such chimeric molecules are provided.

Also provided are cells comprising the nucleic acids or nucleic acid vectors, and cells expressing such chimeric molecules. The chimeric molecules in a fusion protein form may be expressed and isolated using the methods disclosed herein.

In some embodiments, such enhanced binding is also specific to a presynaptic nerve terminal. In some embodiments, the presynaptic nerve terminal is in a mammal. In some embodiments, the presynaptic nerve terminal is in a rodent. In some embodiments, the presynaptic nerve terminal is a mouse presynaptic nerve terminal. In some embodiments, the presynaptic nerve terminal is a mouse presynaptic nerve terminal. In some embodiments, the presynaptic nerve terminal is a human presynaptic nerve terminal.

A modified BoNT polypeptide that has enhanced binding affinity to its target cells (e.g., neurons) affords potential for therapeutic use. For example, such modified BoNT polypeptide may be effective at a lower dose. A lower BoNT dose for therapeutic use is generally desirable because less toxin will diffuse to surrounding tissues at the injection site and less neutralizing antibodies may be generated against the BoNT.

Thus, the present disclosure also contemplates pharmaceutically compositions comprising the modified BoNTs or the chimeric molecules of the present disclosure. As it may also become clear later in the present disclosure, the pharmaceutical composition of the present disclosure, may further comprise other therapeutic agents suitable for the specific disease such composition is designed to treat. In some embodiments, the pharmaceutically composition of the present disclosure further comprises pharmaceutically-acceptable carriers.

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the polypeptide from one site (e.g., the delivery site) of the body, to another site (e.g., organ, tissue or portion of the body).

A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethylcellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, a modified BoNT polypeptide of the present disclosure in a composition is administered by injection, by means of a catheter, by means of a suppository, or by means of an implant, the implant being of a porous, non-porous, or gelatinous material, including a membrane, such as a sialastic membrane, or a fiber.

Typically, when administering the composition, materials to which the polypeptide of the disclosure does not absorb are used. In other embodiments, the modified BoNT polypeptides of the present disclosure are delivered in a controlled release system. Such compositions and methods for administration are provides in U.S. Patent publication No. 2007/0020295, the contents of which are herein incorporated by reference. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The modified BoNT polypeptides of the present disclosure can be administered as pharmaceutical compositions comprising a therapeutically effective amount of a binding agent and one or more pharmaceutically compatible ingredients. In typical embodiments, the pharmaceutical composition is formulated in accordance with routine procedures as a pharmaceutical composition adapted for intravenous or subcutaneous administration to a subject, e.g., a human being.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration. A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein.

The polypeptides of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757. The pharmaceutical compositions of the present disclosure may be administered or packaged as a unit dose, for example.

The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle. In some embodiments, the modified BoNT polypeptides described herein may be conjugated to a therapeutic moiety, e.g., an antibiotic. Techniques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158. Further, the pharmaceutical composition can be provided as a pharmaceutical kit comprising (a) a container containing a polypeptide of the disclosure in lyophilized form and (b) a second container containing a pharmaceutically acceptable diluent (e.g., sterile water) for injection. The pharmaceutically acceptable diluent can be used for reconstitution or dilution of the lyophilized polypeptide of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration. In another aspect, an article of manufacture containing materials useful for the treatment of the diseases described above is included. In some embodiments, the article of manufacture comprises a container and a label.

Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an isolated polypeptide of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

The modified BoNT polypeptides, the chimeric molecules, and the pharmaceutical compositions of the present disclosure may be used for the treatment of conditions associated with unwanted neuronal activities. Thus, further provided herein are methods of treating a condition associated with unwanted neuronal activity, the method comprising administering a therapeutically effective amount of the modified BoNT polypeptide, the chimeric molecule, or the pharmaceutical composition described herein to thereby treat the condition. In some embodiments, the modified BoNT polypeptides, the chimeric molecules, and the pharmaceutic compositions of the present disclosure contact one or more neuron(s) exhibiting unwanted neuronal activity,

Condition typically treated with a neurotoxin (e.g., skeletal muscle conditions, smooth muscle conditions, glandular conditions, a neuromuscular disorder, an autonomic disorder, pain, or an aesthetic/cosmetic condition) are associated with unwanted neuronal activity, as determined by the skilled practitioner. Administration is by a route that contacts an effective amount of the composition to neurons exhibiting the unwanted activity. In some embodiments, the condition may be associated with overactive neurons or glands. Specific conditions envisioned for treatment by the methods discussed herein include, without limitation, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions as well as other secretory disorders, pain from muscle spasms, headache pain. In addition, the present disclosure can be used to treat dermato logical or aesthetic/cosmetic conditions, for example, reduction of brow furrows, reduction of skin wrinkles.

The present disclosure can also be used in the treatment of sports injuries. Borodic U.S. Pat. No. 5,053,005 discloses methods for treating juvenile spinal curvature, i.e. scoliosis, using botulinum type A. The disclosure of Borodic is incorporated in its entirety herein by reference. In one embodiment, using substantially similar methods as disclosed by Borodic, a modified neurotoxin can be administered to a mammal, preferably a human, to treat spinal curvature. In a suitable embodiment, a modified neurotoxin comprising botulinum type E fused with a leucine-based motif is administered. Even more preferably, a modified neurotoxin comprising botulinum type A-E with a leucine-based motif fused to the carboxyl terminal of its light chain is administered to the mammal, preferably a human, to treat spinal curvature.

In addition, the modified neurotoxin can be administered to treat other neuromuscular disorders using well known techniques that are commonly performed with botulinum type A. For example, the present disclosure can be used to treat pain, for example, headache pain, pain from muscle spasms and various forms of inflammatory pain. For example, Aoki U.S. Pat. No. 5,721,215 and Aoki U.S. Pat. No. 6,113,915 disclose methods of using botulinum toxin type A for treating pain. The disclosure of these two patents is incorporated in its entirety herein by reference.

Autonomic nervous system disorders can also be treated with a modified neurotoxin. For example, glandular malfunctioning is an autonomic nervous system disorder. Glandular malfunctioning includes excessive sweating and excessive salivation. Respiratory malfunctioning is another example of an autonomic nervous system disorder. Respiratory malfunctioning includes chronic obstructive pulmonary disease and asthma. Sanders et al. disclose methods for treating the autonomic nervous system; for example, treating autonomic nervous system disorders such as excessive sweating, excessive salivation, asthma, etc., using naturally existing botulinum toxins. The disclosure of Sander et al. is incorporated in its entirety by reference herein.

In one embodiment, substantially similar methods to that of Sanders et al. can be employed, but using a modified neurotoxin, to treat autonomic nervous system disorders such as the ones discussed above. For example, a modified neurotoxin can be locally applied to the nasal cavity of the mammal in an amount sufficient to degenerate cholinergic neurons of the autonomic nervous system that control the mucous secretion in the nasal cavity. Pain that can be treated by a modified neurotoxin includes pain caused by muscle tension, or spasm, or pain that is not associated with muscle spasm. For example, Binder in U.S. Pat. No. 5,714,468 discloses that headache caused by vascular disturbances, muscular tension, neuralgia and neuropathy can be treated with a naturally occurring botulinum toxin, for example Botulinum type A. The disclosures of Binder are incorporated in its entirety herein by reference.

In one embodiment, substantially similar methods to that of Binder can be employed, but using a modified neurotoxin, to treat headache, especially the ones caused by vascular disturbances, muscular tension, neuralgia and neuropathy. Pain caused by muscle spasm can also be treated by an administration of a modified neurotoxin. For example, a botulinum type E fused with a leucine-based motif, preferably at the carboxyl terminal of the botulinum type E light chain, can be administered intramuscularly at the pain/spasm location to alleviate pain. Furthermore, a modified neurotoxin can be administered to a mammal to treat pain that is not associated with a muscular disorder, such as spasm.

In one broad embodiment, methods of the present disclosure to treat non-spasm related pain include central administration or peripheral administration of the modified neurotoxin. For example, Foster et al. in U.S. Pat. No. 5,989,545 discloses that a botulinum toxin conjugated with a targeting moiety can be administered centrally (intrathecally) to alleviate pain. The disclosures of Foster et al. are incorporated in its entirety by reference herein.

In one embodiment, substantially similar methods to that of Foster et al. can be employed, but using the compositions described herein to treat pain. The pain to be treated can be an acute pain or chronic pain. An acute or chronic pain that is not associated with a muscle spasm can also be alleviated with a local, peripheral administration of the modified neurotoxin to an actual or a perceived pain location on the mammal.

In one embodiment, the modified neurotoxin is administered subcutaneously at or near the location of pain, for example, at or near a cut. In some embodiments, the modified neurotoxin is administered intramuscularly at or near the location of pain, for example, at or near a bruise location on the mammal. In some embodiments, the modified BoNT polypeptide is injected directly into a joint of a mammal, for treating or alleviating pain caused by arthritic conditions. Also, frequent repeated injection or infusion of the modified neurotoxin to a peripheral pain location is within the scope of the present disclosure. Routes of administration for such methods are known in the art and easily adapted to the methods described herein by the skilled practitioner (e.g., see for example, Harrison's Principles of Internal Medicine (1998), edited by Anthony Fauci et al., 14.sup.th edition, published by McGraw Hill).

By way of non-limiting example, the treatment of a neuromuscular disorder can comprise a step of locally administering an effective amount of the molecule to a muscle or a group of muscles, the treatment of an autonomic disorder can comprise a step of locally administering an effective of the molecule to a gland or glands, and the treatment of pain can comprise a step of administering an effective amount of the molecule the site of the pain. In addition, the treatment of pain can comprise a step of administering an effective amount of a modified neurotoxin to the spinal cord.

“A therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including the polypeptide used) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide (such as the half-life of the polypeptide, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the polypeptide is administered for preventive or therapeutic purposes, previous therapy, the subject's clinical history and response to the antagonist, and the discretion of the attending physician. Typically the clinician will administer a polypeptide until a dosage is reached that achieves the desired result.

Administration of one or more polypeptides can be continuous or intermittent, depending, for example, upon the recipient's physiological condition, whether the purpose of the administration is therapeutic or prophylactic, and other factors known to skilled practitioners. The administration of a polypeptide may be essentially continuous over a preselected period of time or may be in a series of spaced dose, e.g., either before, during, or after developing a disease. As used herein, the term “treating” refers to the application or administration of a polypeptide or composition including the polypeptide to a subject in need thereof.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has CDI. In some embodiments, the subject has cancer. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

As used herein, a “subject” refers to a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human.

The terms, “patient” and “subject” are used interchangeably herein. A subject can be male or female. A subject can be a fully developed subject (e.g., an adult) or a subject undergoing the developmental process (e.g., a child, infant or fetus). Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with unwanted neuronal activity. In addition, the methods and compositions described herein can be used to treat domesticated animals and/or pets.

### Examples

Botulinum neurotoxins are a family of bacterial toxins, including seven major serotypes (BoNT/A-G) ^{1, 2} and recently discovered BoNT/EN and BoNT/X. These toxins act by blocking neurotransmitter release from neurons, thus paralyzing animals and humans. In recent years, BoNTs have been widely used to treat a growing list of medical conditions: local injections of minute amount of toxins can attenuate neuronal activity in targeted regions, which can be beneficial in many medical conditions as well as for cosmetic purposes ³⁻⁵.

BoNT/A and BoNT/B are the only two BoNTs that are currently FDA-approved for use in humans ³⁻⁵. These are toxins purified from bacteria without any sequence modifications (defined as wild type, WT). As the application of BoNTs grows, limitations and adverse effects have been reported. The major limitation is the generation of neutralizing antibodies in patients, which renders future treatment ineffective ⁶. Termination of BoNT usage often leaves patients with no other effective ways to treat or relieve their disorders. The probability of antibody responses is directly related to both toxin doses and the frequency of injection ⁶. Therefore, this limitation mainly occurs in treating muscle spasms, which involves high dose of toxins.

The observed adverse effects of BoNTs are largely due to diffusion of toxins to other regions of the body and the possibility of toxin diffusion is directly related to injected doses. The adverse effects range from transient non-serious events such as ptosis and diplopia to life-threatening events even death ^{7, 8}. In a petition letter filed in 2008 by Dr. Sidney Wolfe to FDA, a total of 180 serious adverse events, including 16 deaths have been documented. As a result, FDA now requires the “Black box warning” on all BoNT products, highlighting the risk of the spread of toxins, following similar warnings issued by the European Union.

Because both the generation of neutralizing antibodies and toxin diffusion are directly related to injected doses, lowering toxin doses (while maintaining the same levels of toxin activity) is highly desired, which means the efficacy of individual toxin molecules to induce local muscle paralysis has to be enhanced. Such modified BoNTs with improved local efficacy will also reduce any potential off-target effects due to toxin diffusion to other regions.

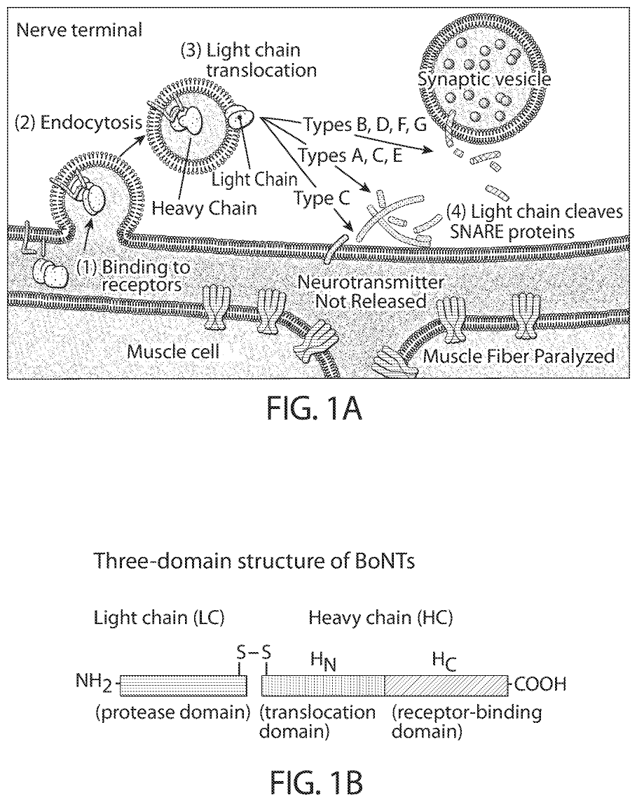

BoNTs have a three-step mechanism (FIG. 1A): (1) receptor binding: these toxins target motor nerve terminals by first binding specifically to their receptors expressed in neurons; (2) translocation: after binding to receptors, BoNTs enter cells via receptor-mediated endocytosis into endosomes, and the low pH conditions within endosomes then induce conformational changes of toxin, resulting in its penetration of endosomal membrane and release of its protease domain into the cytosol of neurons; (3) substrate cleavage: within the cytosol of neurons, the released protease domain of BoNTs then cleave proteins required for synaptic transmission, therefore blocking neurotransmission 2. Corresponding to these three steps of action, BoNTs are composed of three functional domains (FIG. 1B) 2: (1) the C-terminal receptor-binding domain (H_{C}, ˜50 kDa); (2) the membrane translocation domain in the middle (H_{N}, ˜50 kDa); (3) the N-terminal protease domain (also known as light chain, LC, ˜50 kDa). The H_{C} and H_{N} together forms the heavy chain (HC, ˜100 kDa).

Receptor-binding appears to be a rate-limiting step. For instance, enhancing the ability of BoNTs to recognize their neuronal receptors will facilitate absorbance of toxins into neurons at the injection site, therefore shielding toxins from triggering immune responses and also preventing their diffusion. Enhanced affinity and specificity to neuronal receptors will also reduce potential off-target effects due to non-specific entry into other cell types. The receptors for most BoNTs have been identified in recent years. BoNT/B, G, and a mosaic toxin DC share two homologous synaptic vesicle proteins synaptotagmin I and II (Syt I/II) as their receptors ⁹⁻¹⁶. Another family of synaptic vesicle protein SV2 acts as receptors for BoNT/A, E, D, and potentially F ^{10, 17-22}. In addition to protein receptors, all BoNTs require lipid co-receptor gangliosides, which are abundant on neuronal surfaces ²³.

Recent progresses in genomic sequencing revealed that there are a growing number of subtype sequences within each major serotype of BoNTs ²⁴. For instance, BoNT/A currently contains at least 8 subtypes (A1-A8), with a total of 37 distinct sequences (bontbase.org). There are as much as 15% differences at protein levels among these subtypes. These differences may not alter the receptor or basic function of the toxin, but may have significant impacts on binding affinity to receptors, translocation efficacy, protein stability in serum, etc. For example, BoNT/A2 has been shown to have a higher level of efficacy on cultured neurons compared with the standard BoNT/A1 (which is BOTOX®) ²⁵. Therefore, they offer a vast resource to explore residue changes that may improve the overall efficacy of a toxin. However, these residue changes occurred randomly in nature. Although some of the changes are beneficial for the toxin and improving its efficacy, many other residue changes may simultaneously reduce the efficacy of the toxin.

Presented herein is a consensus mutagenesis approach wherein all available BoNT/A subtype sequences were compared, and the most commonly appearing residues at a certain position within the H_{C} were selected, with the assumption that these residues were optimal for that position as it was preserved in most BoNT/A sequences. Second, available H_{C} of BoNT/A1 (H_{C}/A1) complex with its receptor glycosylated SV2 26 was used to design specific mutations aiming to enhance binding of H_{C}/A1 to glycosylated SV2.

### Rational Design of Consensus Mutagenesis of H_{C}/A1.

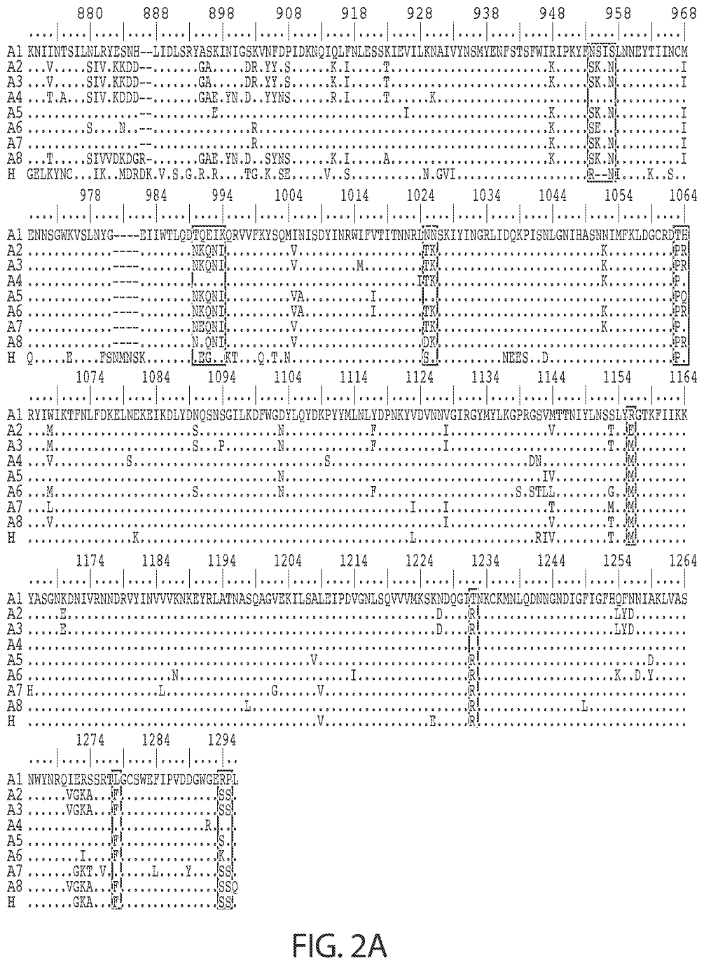

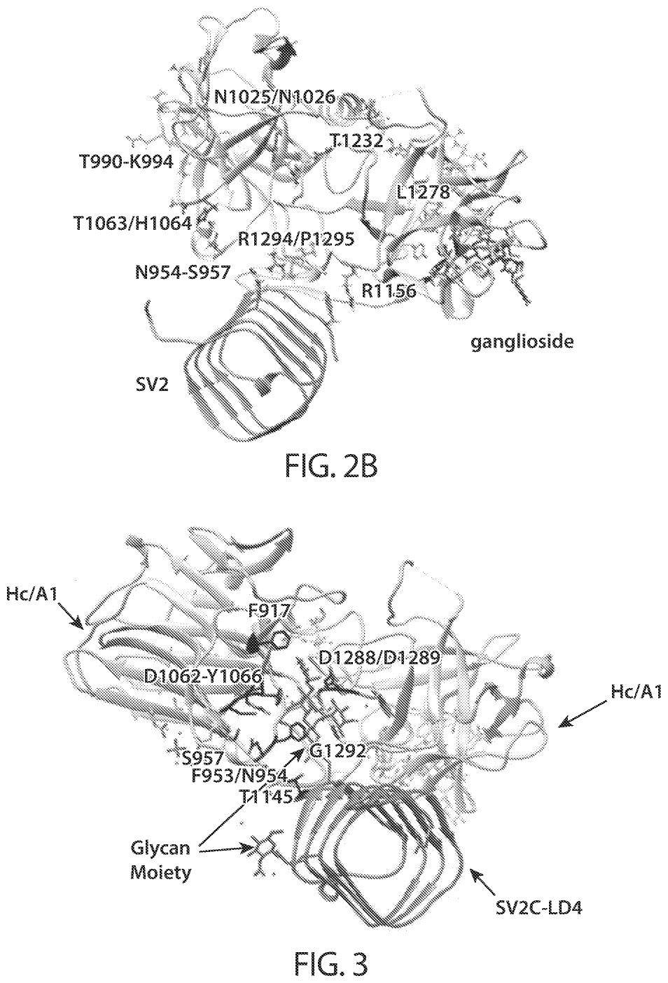

First, H_{C}/A1 sequences were compared with all other BoNT/A subtypes (A1-A8). The H_{C} of BoNT/HA, a recently discovered chimeric toxin containing a H_{C} with over 80% identity with A1, was also included (FIG. 2A) ²⁷. From this sequence comparison, a total of 23 positions (FIG. 2A and Table 1) were identified based on a residue with distinct properties that appeared in other subtypes compared with A1.

TABLE 1
Mostly conserved residues in A subtypes, but not in A1.

| Sequence in A1 | Major in other subtypes |
|---|---|
| R948 | K |
| N954 | S |
| S955 | K |
| S957 | N |
| M968 | I |
| T990 | N |
| Q991 | K |
| E992 | Q |
| I993 | N |
| K994 | I |
| I1005 | V |
| N1025 | T |
| N1026 | K |
| N1052 | K |
| T1063 | P |
| H1064 | R |
| R1156 | M |
| T1232 | R |
| E1272 | G |
| R1273 | K |
| L1278 | F |
| R1294 | S |
| P1295 | S |

Among these 23 positions, 17 positions were selected for experimental validation based on their high frequency appearing in multiple subtypes and also excluding the ones with similar residues (e.g. R versus K) (marked with boxes in FIG. 2A, Table 2). A few of these residues are next to each other and they were mutated at the same time. In total, nine mutants were selected (Table 2). The location of these 17 mutation sites were marked in the structure of HC/A (FIG. 2B).

TABLE 2
Selected 9 consensus mutants for evaluation

| Consensus mutations | Mimicking subtypes |
|---|---|
| N954S/S955K/S957N | A2, A3, A5, A7, A8 |
| T990N/Q991K/E992Q/I993N/K994I | A2, A3, A5, A6, A8 |
| N1025T/N1026K | A2, A3, A4, A6, A7 |
| T1063P/H1064R | A2, A3, A6, A8 |
| R1156M | A3, A4, A5, A6, A7, A8, H |
| T1232R | A2, A3, A5, A6, A7, A8, H |
| L1278F | A2, A3, A5, A6, A8, H |
| R1294S/P1295S | A2, A3, A7, A8, H |

### Rationale Design of H_{C}/A1 Mutations to Enhance Binding to Glycosylated SV2

The co-crystal structure of H_{C}/A1 in complex with glycosylated human SV2C shows that H_{C}/A1 binds to mainly the N-linked glycan via extensive contacts (FIG. 3) ²⁶. The protein-protein interactions between H_{C}/A1 and SV2C are mainly through backbone to backbone interactions, which lack the specificity of side-chain mediated interactions. Therefore, the binding to the N-linked glycan was enhanced by altering key residues that formed contacts with the glycan. A total of 19 mutations were designed (FIG. 3, Table 3).

TABLE 3
Designed mutations for enhancing glycan binding
Mutations for glycan binding

F917R
F917K
F953H
F953Y
N954Q
S957Q
S957Y
D1062E
T1063P
H1064Q
R1065N
Y1066R
Y1066K
T1145Y
D1288E
D1288N
D1289Y
G1292R
G1292K

Seven mutation of the mutations in Table 3 were selected for experimental validation because they contained natural variations at these positions among BoNT/A subtypes (Table 4).

TABLE 4
Evaluated mutations for glycan binding variants
Mutations for glycan binding

F917R
F917K
N954Q
S957Q
T1063P
H1064Q
D1289Y

### Experimental Validation of BoNT/A1 Mutations

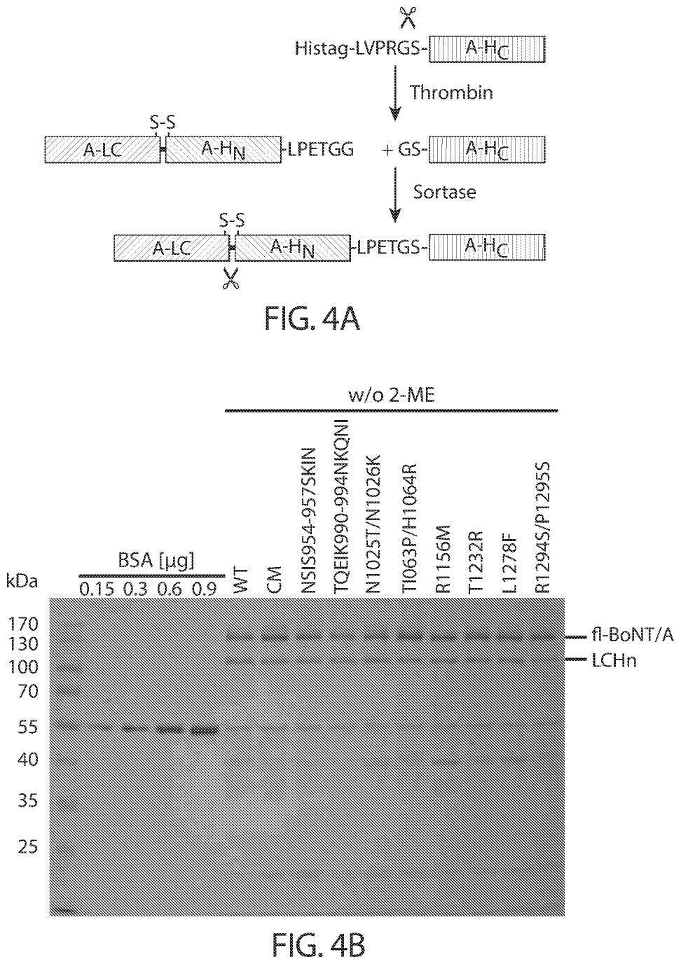

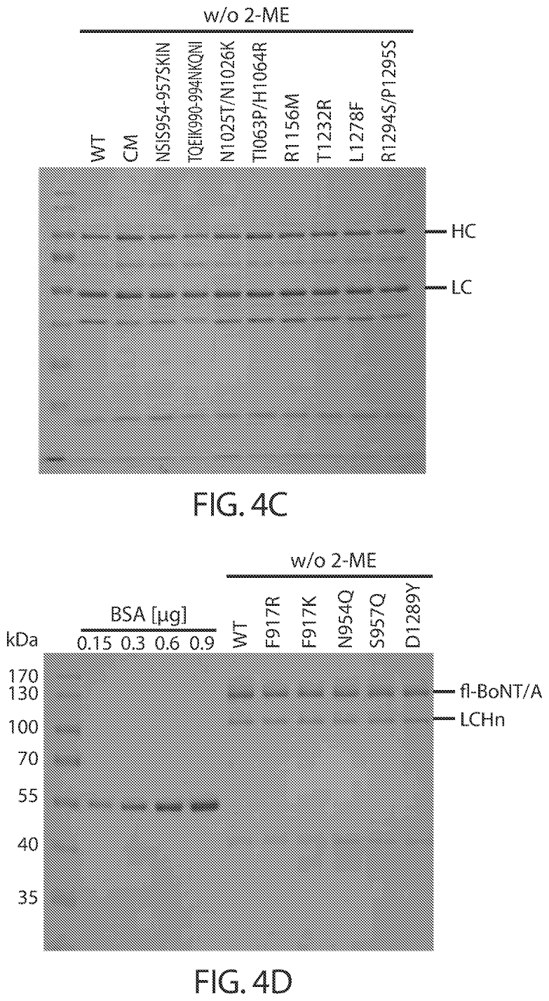

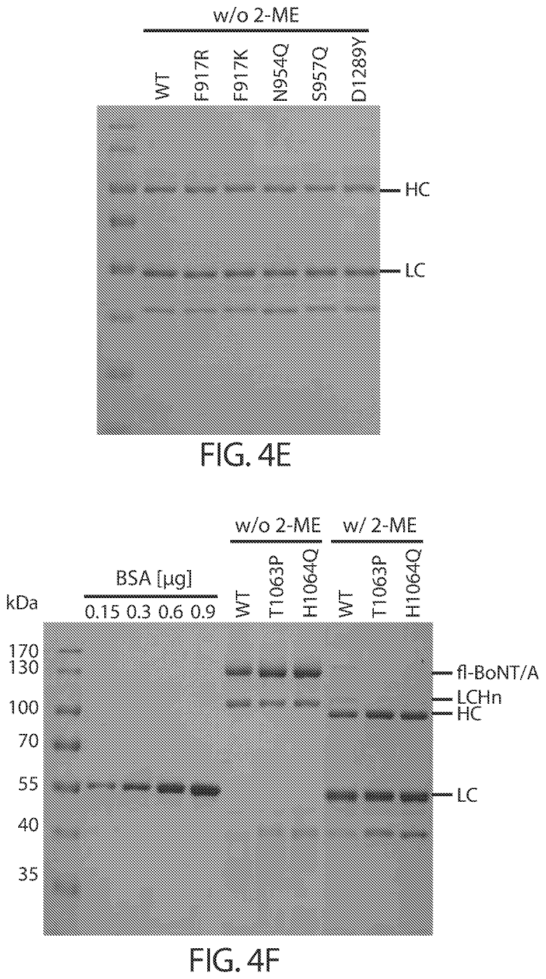

The selected mutations were evaluated to determine whether they would enhance or alter the overall efficacy of BoNT/A1 over the natural form in vivo. To ensure biosafety, cDNA sequences that encoded full-length toxin were not produced and live organisms that could harbor the full toxin gene were not developed. Instead, a sortase-mediated ligation method was used (FIG. 4A) ^{28, 29}. This method relied on producing two separated pieces of BoNTs: the LC-H_{N} fragment with a sortase recognition site at the C-terminus; and the H_{C} with a free glycine at its N-terminus. Each piece alone was not toxic. Then, the two proteins were ligated together biochemically by adding sortase, a bacterial transpeptidase, which recognizes the sortase tag (LPETG) and ligates it to the glycine at the N-terminal of H_{C}, thus producing a full-length toxin protein, without ever having the coding sequence for the full-length toxin. As a result, the amount of toxins produced in the test tube could be precisely controlled to ensure that the total amount was below the exempted amount for BoNTs (<1 mg).

Wild type (WT) H_{C}/A1 and mutant H_{C}/A1 proteins containing point mutations described in Table 2 and Table 4 were produced in E. coli, and then ligated to the same LC-HN/A1 fragment derived from BoNT/A1, generating full toxins at ˜150 kDa (FIG. 4B, FIG. 4D). BoNTs are produced as a single polypeptide. The linker region between LC and H_{N} needs to be separated by proteases in order to release LC upon entry into the cytosol. This process is known as “activation.” Activated LC-H_{N} remains connected by a disulfide bond before reaching the cytosol. To facilitate the activation step, an extra thrombin cleavage site was introduced between LC and HN. Treatment of thrombin resulted in almost compete activation of these ligated toxins (FIG. 4C, FIG. 4E-F).

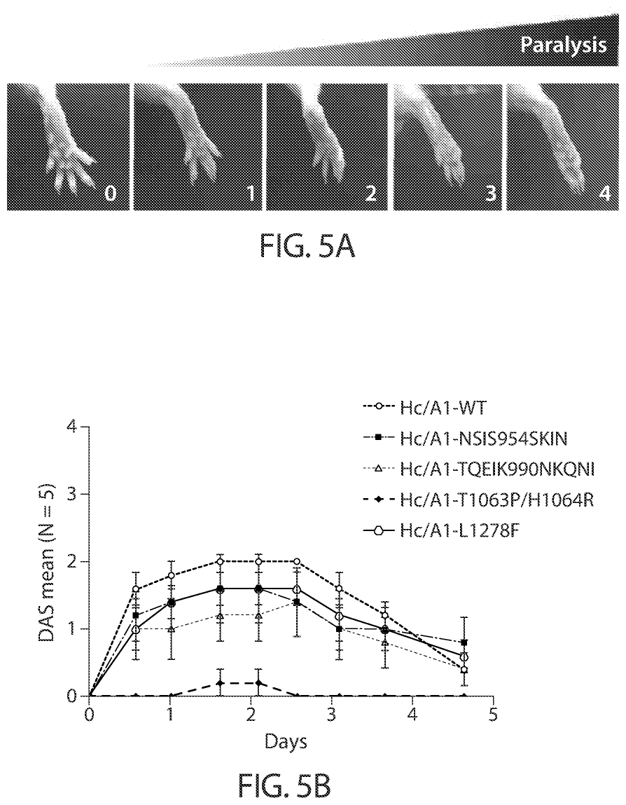

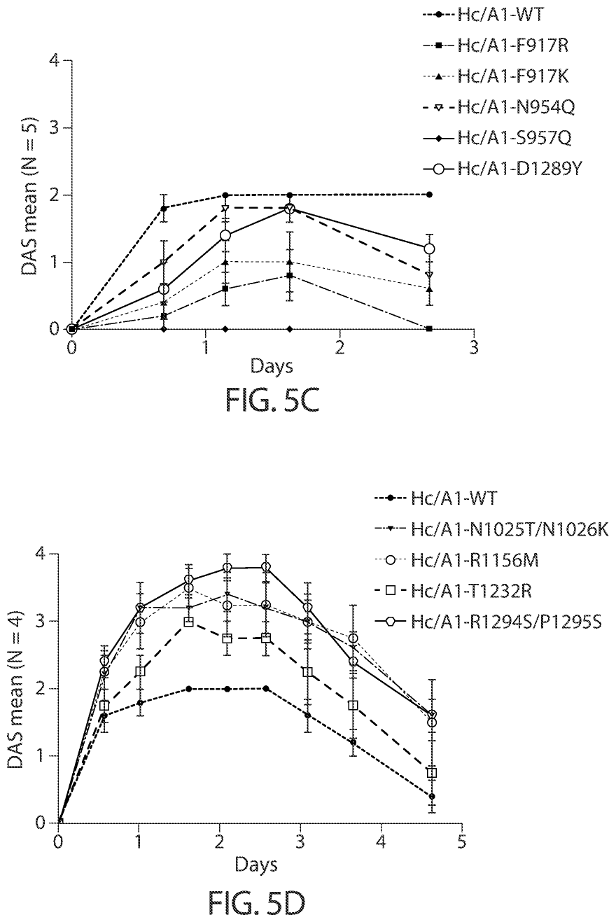

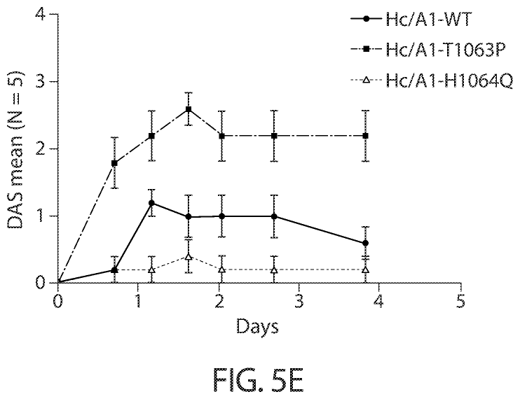

The ligated toxins were evaluated for their efficacy in inducing local muscle paralysis using a well-established mouse Digit Abduction Score assay ³⁰. In this assay, a tiny amount of ligated toxin was injected into the hind leg of mice. Paralysis of this leg leads to loss of ability to spread the toe during startle response. The degree of toe spread reflects the degree of muscle paralysis, and can be quantified on the scale of 0 (no paralysis) to 4 (severest paralysis) (FIG. 5A). Compared with the same dose of wild type (WT), the T1063P/H1064R combination clearly and consistently induced a lower degree of paralysis (FIG. 5B), indicating that this combination was detrimental to BoNT/A1. Similarly, F917R, F917K, N954Q, S957Q, and D1289Y all showed lower activity than WT toxin (FIG. 5C). Many other mutations such as NSIS954-957SKINTQEIK990-994NKQNI, and L1278F showed no significant impact on the degree of paralysis (FIG. 5B). Interestingly, N1025T/N1026K, R1156M, T1232R, R1294S/P1295S all showed higher degree of paralysis than WT (FIG. 5D), suggesting that these residue changes enhanced the overall efficacy of BoNT/A1 in inducing local muscle paralysis. DAS of T1063P and H1064 were measured along side wildtype H_{C}/A1-WT (FIG. 5E). T1063P showed higher scores than WT, while H1064Q showed reduced activity compared to WT.

The activity of detrimental and neutral mutations such as T1063P/H1064R, NSIS954-957SKIN and TQEIK990-994NKQNI was examined at a high dose (30 pg). At this high dose, toxins diffused to other regions and induced body weight reduction. If body weight reduction reached >20%, mice were euthanized due to safety protocols. Each mouse was weighed and their DAS scores were recorded to determine the degree of toxin diffusion. WT toxin at 30 pg resulted in score 4 and >20% body weight reduction. T1063P/H1064R, NSIS954-957SKIN, and TQEIK990-994NKQNI induced score 4 paralysis similar to WT toxin, (Table 5).

TABLE 5
Maximum DAS scores and weight loss for BoNT
injected mice with 30 pg dose (n > 2).
Mice with >20% body weight loss were
euthanized according to safety protocol.

| | Maximum | Loss of |
|---|---|---|
| Mutants | DAS score | weight, % |
| Hc/A1-WT | 4 | >20 |
| Hc/A1-CM | 3 | 7.3 |
| Hc/A1-NSIS954-957SKIN | 4 | 10.3 |
| Hc/A1-TQEIK990-994NKQNI | 4 | 19.4 |
| Hc/A1-N1025T/N1026K | 4 | >20 |
| Hc/A1-T1063P/H1064R | 4 | 2.5 |
| Hc/A1-R1156M | 4 | >20 |
| Hc/A1-T1232R | 4 | >20 |
| Hc/A1-L1278F | 4 | 17.9 |
| Hc/A1-R1294S/P1295S | 4 | >20 |

Interestingly, T1063P/H1064R, NSIS954-957SKIN, and L1278F showed less reduction in body weight (2.5% for T1063P/H1064R, 10.3% for NSIS954-957SKIN, and 17.9% for L1278F) than WT toxin, and no mice needed euthanization. Although these mutants showed reduced potency when compared to WT toxins, the data suggests that they have reduced systemic toxicity at a dosage that induces the same degree of paralysis as WT toxin. Thus, mutations at these sites also confer beneficial properties of reduced diffusion compared with WT H_{C}/A1.

### Rational Design of Consensus Mutagenesis of H_{C}/A1

Positions for consensus mutagenesis of H_{C}/A2 were also identified (Table 6 and Table 7).

TABLE 6
Consensus mutations for Hc/A2.

| Sequence in A2 | Majority in other subtypes |
|---|---|
| K915 | Q |
| T923 | K |
| S1090 | N |
| N1103 | D |
| F1117 | Y |
| E1156 | M |
| E1170 | K |
| D1227 | N |
| L1254 | Q |
| Y1255 | F |
| D1256 | N |

TABLE 7
Potential effects of each tested mutation for Hc/A1, which might
improve efficacy or reduce diffusion and systemic toxicity.

| | Potential effects | |
|---|---|---|
| Consensus mutations | Improving efficacy | Reducing diffusion |
| N954S/S955K/S957N | | ◯ |
| N1025T/N1026K | ◯ | |
| T1063P/H1064R | | ◯ |
| R1156M | ◯ | |
| T1232R | ◯ | |
| R1294S/P1295S | ◯ | |
| L1278F | | ◯ |

### Materials and Methods

Materials: All protein biomaterials were reconstituted in Tris-buffered saline (TBS: 50 mM Tris, 150 mM NaCl, pH7.5) unless indicated otherwise.

cDNA and constructs: DNA encoding BoNT/A1-LC-H_{N} and BoNT/A1-H_{C} (residue 1-875 and residue 882-1296, respectively based on GenBank access No:ACS52162.1) were synthesized by Genewiz Inc., and its codon was optimized for expression in E. coli. The construct of LC-H_{N} contained an artificial linker with a thrombin cleavage site (ASLVPRGSGGSAAA) (SEQ ID NO: 124) between residue 441 and 442. DNA encoding the recombinant proteins was subcloned into pET28a vectors to contain both a His6 tag and a thrombin cleavage site (LVPRGS) (SEQ ID NO: 125) fused to the N-terminus of BoNT/A1-H_{C}, and both a sortase tag (LPETG) (SEQ ID NO: 126) and a His6 tag fused to the C-terminus of BoNT/A1-LC-HN. Mutations in BoNT/A1-H_{C} were generated via PCR using a pair of primers containing each mutation. All constructs were verified by sequencing.

Protein expression and purification: WT and mutants of BoNT/A1-H_{C} were expressed as His6 tagged recombinant proteins in E. coli BL21(DE3) strain with the induction temperature at 18° C. overnight in the autoinduction medium (Formedium, Ltd.). His6-fusion proteins were purified using Ni-NTA bead charged column. Briefly, bacterial lysates were incubated with Ni-NTA beads, followed by flow-through of washing buffer (40 mM imidazole, TBS, pH7.5) and elution buffer (100-500 mM imidazole, TBS, pH7.5). Eluted proteins were desalted and digested with thrombin Sepharose bead (BioVision, Inc.). Inactivated proteins were removed using prewashed Ni-NTA beads, and supernatant (activated proteins) was concentrated, quantified and frozen (−80° C.) until use.

Sortase ligation: 4 μM of substrate proteins, LC-H_{N} and H_{C} were premixed with TBS, pH 7.5 containing 10 mM CaCl₂. 0.5 μM of recombinant sortase was added and incubated for 40 minutes at room temperature. Prewashed Ni-NTA beads were loaded to remove His-tagged sortase and unligated substrate proteins. Supernatant was only activated using 25 U/ml thrombin (Millipore) for 40 minutes at room temperature. Some of ligated products were analyzed on the SDS-PAGE gel with or without 2-mercaptoethanol to confirm ligation and activation. Full-length toxins were quantified based on protein density on the gel using BSA as a standard (0.15-0.9 μg).

Digital Abduction Score (DAS) assay: The control BoNT/A1 WT and mutant toxins (ligated and thrombin-activated full-length toxins) were reconstituted in phosphate buffer containing 0.2% gelatin (pH 6.3), and serial dilutions were prepared based on preliminary dose-response analysis. The following final concentrations were tested in DAS assay: 5, 15 and 30 pg/mouse (CD-1 strain, Envigo). The 10 μl diluted toxins were injected into the right gastrocnemius muscle of each mouse. Their scores were monitored according to FIG. 5A.

TABLE 8
Amino acid sequences

| SEQ | | |
|---|---|---|
| ID | | |
| NO: | Description | Sequence |
| 1 | BoNT/ | MPFVNKQFNYKDPVNGVDIAYIKIPNAGQM |
| | A1 | QPVKAFKIHNKIWVIPERDTFTNPEEGDLN |
| | WT Full | PPPEAKQVPVSYYDSTYLSTDNEKDNYLKG |
| | length | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 2 | BoNT/ | MPFVNKQFNYKDPVNGVDIAYIKIPNAGQM |
| | A2 | QPVKAFKIHNKIWVIPERDTFTNPEEGDLN |
| | WT Full | PPPEAKQVPVSYYDSTYLSTDNEKDNYLKG |
| | length | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 3 | BoNT/ | MPFVNKQFNYKDPVNGVDIAYIKIPNAGQM |
| | A1-F917R | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLRNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 4 | BoNT/ | MPFVNKQFNYKDPVNGVDIAYIKIPNAGQM |
| | A1-F917K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLKNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 5 | BoNT/ | MPFVNKQFNYKDPVNGVDIAYIKIPNAGQM |
| | A1-F953H | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYHNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 6 | BoNT/ | MPFVNKQFNYKDPVNGVDIAYIKIPNAGQM |
| | A1-F953Y | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYYNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 7 | BoNT/ | MPFVNKQFNYKDPVNGVDIAYIKIPNAGQM |
| | A1-N954S | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFSSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 8 | BoNT/ | MPFVNKQFNYKDPVNGVDIAYIKIPNAGQM |
| | A1-S955K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNKISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 9 | BoNT/ | MPFVNKQFNYKDPVNGVDIAYIKIPNAGQM |
| | A1-S957N | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSINLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 10 | BoNT/ | MPFVNKQFNYKDPVNGVDIAYIKIPNAGQM |
| | A1-M9681 | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCIENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 11 | BoNT/ | MPFVNKQFNYKDPVNGVDIAYIKIPNAGQM |
| | A1-N1025T | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLTNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 12 | BoNT/ | MPFVNKQFNYKDPVNGVDIAYIKIPNAGQM |
| | A1-N1026K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNKSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 13 | BoNT/ | MPFVNKQFNYKDPVNGVDIAYIKIPNAGQM |
| | A1-N1052K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NKIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 14 | BoNT/ | MPFVNKQFNYKDPVNGVDIAYIKIPNAGQM |
| | A1-D1062E | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRETHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 15 | BoNT/ | MPFVNKQFNYKDPVNGVDIAYIKIPNAGQM |
| | A1-T1063P | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDPHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 16 | BoNT/ | MPFVNKQFNYKDPVNGVDIAYIKIPNAGQM |
| | A1-H1064R | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTRRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 17 | BoNT/ | MPFVNKQFNYKDPVNGVDIAYIKIPNAGQM |
| | A1-H1064Q | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTQRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 18 | BoNT/ | MPFVNKQFNYKDPVNGVDIAYIKIPNAGQM |
| | A1-R1065N | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHNYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 19 | BoNT/ | MPFVNKQFNYKDPVNGVDIAYIKIPNAGQM |
| | A1-Y1066R | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRRIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 20 | BoNT/ | MPFVNKQFNYKDPVNGVDIAYIKIPNAGQM |
| | A1-Y1066K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRKIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 21 | BoNT/ | MPFVNKQFNYKDPVNGVDIAYIKIPNAGQM |
| | A1-T1145Y | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMYTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 22 | BoNT/ | MPFVNKQFNYKDPVNGVDIAYIKIPNAGQM |
| | A1-R1156M | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYMGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 23 | BoNT/ | MPFVNKQFNYKDPVNGVDIAYIKIPNAGQM |
| | A1-R11561 | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYIGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERPL |
| 24 | BoNT/ | MPFVNKQFNYKDPVNGVDIAYIKIPNAGQM |
| | A1-T1232R | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | IRNKCKMNLQDNNGNDIGFIGFHQFNNIAK |
| | | LVASNWYNRQIERSSRTLGCSWEFIPVDDG |
| | | WGERPL |
| 25 | BoNT/ | MPFVNKQFNYKDPVNGVDIAYIKIPNAGQM |
| | A1-T1232K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | IKNKCKMNLQDNNGNDIGFIGFHQFNNIAK |
| | | LVASNWYNRQIERSSRTLGCSWEFIPVDDG |
| | | WGERPL |
| 26 | BoNT/ | MPFVNKQFNYKDPVNGVDIAYIKIPNAGQM |
| | A1-E1272G | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIGRSSRTLGCSWEFIPVDDG |
| | | WGERPL |
| 27 | BoNT/ | MPFVNKQFNYKDPVNGVDIAYIKIPNAGQM |
| | A1-L1278F | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTFGCSWEFIPVDDG |
| | | WGERPL |
| 28 | BoNT/ | MPFVNKQFNYKDPVNGVDIAYIKIPNAGQM |
| | A1-L1278Y | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTYGCSWEFIPVDDG |
| | | WGERPL |
| 29 | BoNT/ | MPFVNKQFNYKDPVNGVDIAYIKIPNAGQM |
| | A1-L1278W | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTWGCSWEFIPVDDG |
| | | WGERPL |
| 30 | BoNT/ | MPFVNKQFNYKDPVNGVDIAYIKIPNAGQM |
| | A1-D1288E | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVEDG |
| | | WGERPL |
| 31 | BoNT/ | MPFVNKQFNYKDPVNGVDIAYIKIPNAGQM |
| | A1-D1289Y | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDYG |
| | | WGERPL |
| 32 | BoNT/ | MPFVNKQFNYKDPVNGVDIAYIKIPNAGQM |
| | A1-G1292R | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WRERPL |
| 33 | BoNT/ | |
| | A1-G1292K | MPFVNKQFNYKDPVNGVDIAYIKIPNAGQM |
| | | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WKERPL |
| 34 | BoNT/ | MPFVNKQFNYKDPVNGVDIAYIKIPNAGQM |
| | A1-R1294S | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGESPL |
| 35 | BoNT/ | MPFVNKQFNYKDPVNGVDIAYIKIPNAGQM |
| | A1-R1294T | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGETPL |
| 36 | BoNT/ | MPFVNKQFNYKDPVNGVDIAYIKIPNAGQM |
| | A1-P1295S | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERSL |
| 37 | BoNT/ | MPFVNKQFNYKDPVNGVDIAYIKIPNAGQM |
| | A1-P1295T | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHEVLNLTRNGY |
| | | GSTQYIRFSPDFTFGFEESLEVDTNPLLGA |
| | | GKFATDPAVTLAHELIHAGHRLYGIAINPN |
| | | RVFKVNTNAYYEMSGLEVSFEELRTFGGHD |
| | | AKFIDSLQENEFRLYYYNKFKDIASTLNKA |
| | | KSIVGTTASLQYMKNVFKEKYLLSEDTSGK |
| | | FSVDKLKFDKLYKMLTEIYTEDNFVKFFKV |
| | | LNRKTYLNFDKAVFKINIVPKVNYTIYDGF |
| | | NLRNTNLAANFNGQNTEINNMNFTKLKNFT |
| | | GLFEFYKLLCVRGIITSKTKSLDKGYNKAL |
| | | NDLCIKVNNWDLFFSPSEDNFTNDLNKGEE |
| | | ITSDTNIEAAEENISLDLIQQYYLTFNFDN |
| | | EPENISIENLSSDIIGQLELMPNIERFPNG |
| | | KKYELDKYTMFHYLRAQEFEHGKSRIALTN |
| | | SVNEALLNPSRVYTFFSSDYVKKVNKATEA |
| | | AMFLGWVEQLVYDFTDETSEVSTTDKIADI |
| | | TIIIPYIGPALNIGNMLYKDDFVGALIFSG |
| | | AVILLEFIPEIAIPVLGTFALVSYIANKVL |
| | | TVQTIDNALSKRNEKWDEVYKYIVTNWLAK |
| | | VNTQIDLIRKKMKEALENQAEATKAIINYQ |
| | | YNQYTEEEKNNINFNIDDLSSKLNESINKA |
| | | MININKFLNQCSVSYLMNSMIPYGVKRLED |
| | | FDASLKDALLKYIYDNRGTLIGQVDRLKDK |
| | | VNNTLSTDIPFQLSKYVDNQRLLSTFTEYI |
| | | KNIINTSILNLRYESNHLIDLSRYASKINI |
| | | GSKVNFDPIDKNQIQLFNLESSKIEVILKN |
| | | AIVYNSMYENFSTSFWIRIPKYFNSISLNN |
| | | EYTIINCMENNSGWKVSLNYGEIIWTLQDT |
| | | QEIKQRVVFKYSQMINISDYINRWIFVTIT |
| | | NNRLNNSKIYINGRLIDQKPISNLGNIHAS |
| | | NNIMFKLDGCRDTHRYIWIKYFNLFDKELN |
| | | EKEIKDLYDNQSNSGILKDFWGDYLQYDKP |
| | | YYMLNLYDPNKYVDVNNVGIRGYMYLKGPR |
| | | GSVMTTNIYLNSSLYRGTKFIIKKYASGNK |
| | | DNIVRNNDRVYINVVVKNKEYRLATNASQA |
| | | GVEKILSALEIPDVGNLSQVVVMKSKNDQG |
| | | ITNKCKMNLQDNNGNDIGFIGFHQFNNIAK |
| | | LVASNWYNRQIERSSRTLGCSWEFIPVDDG |
| | | WGERTL |
| 38 | BoNT/ | MPFVNKQFNYKDPVNGVDIAYIKIPNAGQM |
| | A2-K915Q | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIQLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 39 | BoNT/ | MPFVNKQFNYKDPVNGVDIAYIKIPNAGQM |
| | A2-T923K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSKIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 40 | BoNT/ | MPFVNKQFNYKDPVNGVDIAYIKIPNAGQM |
| | A2-S1090N | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDNQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 41 | BoNT/ | MPFVNKQFNYKDPVNGVDIAYIKIPNAGQM |
| | A2-N1103D | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGDYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 42 | BoNT/ | MPFVNKQFNYKDPVNGVDIAYIKIPNAGQM |
| | | QPVKAFKIHNKIWVIPERDTFTNPEEGDLN |
| | A2-F1117Y | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLYDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 43 | BoNT/ | MPFVNKQFNYKDPVNGVDIAYIKIPNAGQMQ |
| | A2-E1156M | PVKAFKIHNKIWVIPERDTFTNPEEGDLNP |
| | | PPEAKQVPVSYYDSTYLSTDNEKDNYLKGV |
| | | TKLFERIYSTDLGRMLLTSIVRGIPFWGGS |
| | | TIDTELKVIDTNCINVIQPDGSYRSEELNL |
| | | VIIGPSADIIQFECKSFGHDVLNLTRNGYG |
| | | STQYIRFSPDFTFGFEESLEVDTNPLLGAG |
| | | KFATDPAVTLAHELIHAEHRLYGIAINPNR |
| | | VFKVNTNAYYEMSGLEVSFEELRTFGGHDA |
| | | KFIDSLQENEFRLYYYNKFKDVASTLNKAK |
| | | SIIGTTASLQYMKNVFKEKYLLSEDTSGKF |
| | | SVDKLKFDKLYKMLTEIYTEDNFVNFFKVI |
| | | NRKTYLNFDKAVFRINIVPDENYTIKDGFN |
| | | LKGANLSTNFNGQNTEINSRNFTRLKNFTG |
| | | LFEFYKLLCVRGIIPFKTKSLDEGYNKALN |
| | | DLCIKVNNWDLFFSPSEDNFTNDLDKVEEI |
| | | TADTNIEAAEENISLDLIQQYYLTFDFDNE |
| | | PENISIENLSSDIIGQLEPMPNIERFPNGK |
| | | KYELDKYTMFHYLRAQEFEHGDSRIILTNS |
| | | AEEALLKPNVAYTFFSSKYVKKINKAVEAF |
| | | MFLNWAEELVYDFTDETNEVTTMDKIADIT |
| | | IIVPYIGPALNIGNMLSKGEFVEAIIFTGV |
| | | VAMLEFIPEYALPVFGTFAIVSYIANKVLT |
| | | VQTINNALSKRNEKWDEVYKYTVTNWLAKV |
| | | NTQIDLIREKMKKALENQAEATKAIINYQY |
| | | NQYTEEEKNNINFNIDDLSSKLNESINSAM |
| | | ININKFLDQCSVSYLMNSMIPYAVKRLKDF |
| | | DASVRDVLLKYIYDNRGTLVLQVDRLKDEV |
| | | NNTLSADIPFQLSKYVDNKKLLSTFTEYIK |
| | | NIVNTSILSIVYKKDDLIDLSRYGAKINIG |
| | | DRVYYDSIDKNQIKLINLESSTIEVILKNA |
| | | IVYNSMYENFSTSFWIKIPKYFSKINLNNE |
| | | YTIINCIENNSGWKVSLNYGEIIWTLQDNK |
| | | QNIQRVVFKYSQMVNISDYINRWIFVTITN |
| | | NRLTKSKIYINGRLIDQKPISNLGNIHASN |
| | | KIMFKLDGCRDPRRYIMIKYFNLFDKELNE |
| | | KEIKDLYDSQSNSGILKDFWGNYLQYDKPY |
| | | YMLNLFDPNKYVDVNNIGIRGYMYLKGPRG |
| | | SVVTTNIYLNSTLYMGTKFIIKKYASGNED |
| | | NIVRNNDRVYINVVVKNKEYRLATNASQAG |
| | | VEKILSALEIPDVGNLSQVVVMKSKDDQGI |
| | | RNKCKMNLQDNNGNDIGFIGFHLYDNIAKL |
| | | VASNWYNRQVGKASRTFGCSWEFIPVDDGW |
| | | GESSL |
| 44 | BoNT/ | MPFVNKQFNYKDPVNGVDIAYIKIPNAGQM |
| | A2-E1170K | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNK |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGESSL |
| 45 | BoNT/ | MPFVNKQFNYKDPVNGVDIAYIKIPNAGQM |
| | A2-D1227N | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKNDQG |
| | | IRNKCKMNLQDNNGNDIGFIGFHLYDNIAK |
| | | LVASNWYNRQVGKASRTFGCSWEFIPVDDG |
| | | WGESSL |
| 46 | BoNT/ | MPFVNKQFNYKDPVNGVDIAYIKIPNAGQM |
| | A2-L1254Q | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHQYDNIAK |
| | | LVASNWYNRQVGKASRTFGCSWEFIPVDDG |
| | | WGESSL |
| 47 | BoNT/ | MPFVNKQFNYKDPVNGVDIAYIKIPNAGQM |
| | A2-Y1255F | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLFDNIAK |
| | | LVASNWYNRQVGKASRTFGCSWEFIPVDDG |
| | | WGESSL |
| 48 | BoNT/ | MPFVNKQFNYKDPVNGVDIAYIKIPNAGQM |
| | A2-D1256N | QPVKAFKIHNKIWVIPERDTFTNPEEGDLN |
| | | PPPEAKQVPVSYYDSTYLSTDNEKDNYLKG |
| | | VTKLFERIYSTDLGRMLLTSIVRGIPFWGG |
| | | STIDTELKVIDTNCINVIQPDGSYRSEELN |
| | | LVIIGPSADIIQFECKSFGHDVLNLTRNGY |
| | | GSTQYIRFSPDFTFGFEESLEVDTNPLLGA |
| | | GKFATDPAVTLAHELIHAEHRLYGIAINPN |
| | | RVFKVNTNAYYEMSGLEVSFEELRTFGGHD |
| | | AKFIDSLQENEFRLYYYNKFKDVASTLNKA |
| | | KSIIGTTASLQYMKNVFKEKYLLSEDTSGK |
| | | FSVDKLKFDKLYKMLTEIYTEDNFVNFFKV |
| | | INRKTYLNFDKAVFRINIVPDENYTIKDGF |
| | | NLKGANLSTNFNGQNTEINSRNFTRLKNFT |
| | | GLFEFYKLLCVRGIIPFKTKSLDEGYNKAL |
| | | NDLCIKVNNWDLFFSPSEDNFTNDLDKVEE |
| | | ITADTNIEAAEENISLDLIQQYYLTFDFDN |
| | | EPENISIENLSSDIIGQLEPMPNIERFPNG |
| | | KKYELDKYTMFHYLRAQEFEHGDSRIILTN |
| | | SAEEALLKPNVAYTFFSSKYVKKINKAVEA |
| | | FMFLNWAEELVYDFTDETNEVTTMDKIADI |
| | | TIIVPYIGPALNIGNMLSKGEFVEAIIFTG |
| | | VVAMLEFIPEYALPVFGTFAIVSYIANKVL |
| | | TVQTINNALSKRNEKWDEVYKYTVTNWLAK |
| | | VNTQIDLIREKMKKALENQAEATKAIINYQ |
| | | YNQYTEEEKNNINFNIDDLSSKLNESINSA |
| | | MININKFLDQCSVSYLMNSMIPYAVKRLKD |
| | | FDASVRDVLLKYIYDNRGTLVLQVDRLKDE |
| | | VNNTLSADIPFQLSKYVDNKKLLSTFTEYI |
| | | KNIVNTSILSIVYKKDDLIDLSRYGAKINI |
| | | GDRVYYDSIDKNQIKLINLESSTIEVILKN |
| | | AIVYNSMYENFSTSFWIKIPKYFSKINLNN |
| | | EYTIINCIENNSGWKVSLNYGEIIWTLQDN |
| | | KQNIQRVVFKYSQMVNISDYINRWIFVTIT |
| | | NNRLTKSKIYINGRLIDQKPISNLGNIHAS |
| | | NKIMFKLDGCRDPRRYIMIKYFNLFDKELN |
| | | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYNNIAK |
| | | LVASNWYNRQVGKASRTFGCSWEFIPVDDG |
| | | WGESSL |
| 49 | WT receptor | IINTSILNLRYESNHLIDLSRYASKINIGS |
| | binding | KVNFDPIDKNQIQLFNLESSKIEVILKNAI |
| | domain (H_{c}) | VYNSMYENFSTSFWIRIPKYFNSISLNNEY |
| | BoNT/ | TIINCMENNSGWKVSLNYGEIIWTLQDTQE |
| | A1- | IKQRVVFKYSQMINISDYINRWIFVTITNN |
| | residues 873- | RLNNSKIYINGRLIDQKPISNLGNIHASNN |
| | 1296 of full | IMFKLDGCRDTHRYIWIKYFNLFDKELNEK |
| | length | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 50 | WT receptor | SLIVNTSILSIVYKKDDLIDLSRYGAKINI |
| | binding | GDRVYYDSIDKNQIKLINLESSTIEVILKN |
| | domain (H_{c}) | AIVYNSMYENFSTSFWIKIPKYFSKINLNN |
| | BoNT/ | EYTIINCIENNSGWKVSLNYGEIIWTLQDN |
| | A2- | KQNIQRVVFKYSQMVNISDYINRWIFVTIT |
| | residues 873- | NNRLTKSKIYINGRLIDQKPISNLGNIHAS |
| | 1296 of full | NKIMFKLDGCRDPRRYIMIKYFNLFDKELN |
| | length | EKEIKDLYDSQSNSGILKDFWGNYLQYDKP |
| | | YYMLNLFDPNKYVDVNNIGIRGYMYLKGPR |
| | | GSVVTTNIYLNSTLYEGTKFIIKKYASGNE |
| | | DNIVRNNDRVYINVVVKNKEYRLATNASQA |
| | | GVEKILSALEIPDVGNLSQVVVMKSKDDQG |
| | | IRNKCKMNLQDNNGNDIGFIGFHLYDNIAK |
| | | LVASNWYNRQVGKASRTFGCSWEFIPVDDG |
| | | WGES |
| 51 | H_{c}-BoNT/ | IINTSILNLRYESNHLIDLSRYASKINIGS |
| | A1- | KVNFDPIDKNQIQLRNLESSKIEVILKNAI |
| | F917R (of full | VYNSMYENFSTSFWIRIPKYFNSISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 52 | H_{c}-BoNT/ | IINTSILNLRYESNHLIDLSRYASKINIGS |
| | A1- | KVNFDPIDKNQIQLKNLESSKIEVILKNAI |
| | F917K (of full | VYNSMYENFSTSFWIRIPKYFNSISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 53 | H_{c}-BoNT/ | IINTSILNLRYESNHLIDLSRYASKINIGS |
| | A1- | KVNFDPIDKNQIQLFNLESSKIEVILKNAI |
| | F953H (of full | VYNSMYENFSTSFWIRIPKYHNSISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 54 | H_{c}-BoNT/ | IINTSILNLRYESNHLIDLSRYASKINIGS |
| | A1- | KVNFDPIDKNQIQLFNLESSKIEVILKNAI |
| | F953Y (of full | VYNSMYENFSTSFWIRIPKYYNSISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 55 | H_{c}-BoNT/ | IINTSILNLRYESNHLIDLSRYASKINIGS |
| | A1- | KVNFDPIDKNQIQLFNLESSKIEVILKNAI |
| | N954S (of full | VYNSMYENFSTSFWIRIPKYFSSISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 56 | H_{c}-BoNT/ | IINTSILNLRYESNHLIDLSRYASKINIGS |
| | A1- | KVNFDPIDKNQIQLFNLESSKIEVILKNAI |
| | S955K (of full | VYNSMYENFSTSFWIRIPKYFNKISLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 57 | H_{c}-BoNT/ | IINTSILNLRYESNHLIDLSRYASKINIGS |
| | A1- | KVNFDPIDKNQIQLFNLESSKIEVILKNAI |
| | S957N (of full | VYNSMYENFSTSFWIRIPKYFNSINLNNEY |
| | length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 58 | H_{c}-BoNT/ | IINTSILNLRYESNHLIDLSRYASKINIGS |
| | A1- | KVNFDPIDKNQIQLFNLESSKIEVILKNAI |
| | M9681 (of full | VYNSMYENFSTSFWIRIPKYFNSISLNNEY |
| | length) | TIINCIENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 59 | H_{c}-BoNT/ | IINTSILNLRYESNHLIDLSRYASKINIGS |
| | A1- | KVNFDPIDKNQIQLFNLESSKIEVILKNAI |
| | N1025T (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLTNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 60 | H_{c}-BoNT/ | IINTSILNLRYESNHLIDLSRYASKINIGS |
| | A1- | KVNFDPIDKNQIQLFNLESSKIEVILKNAI |
| | N1026K (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNKSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 61 | H_{c}-BoNT/ | IINTSILNLRYESNHLIDLSRYASKINIGS |
| | A1- | KVNFDPIDKNQIQLFNLESSKIEVILKNAI |
| | N1052K (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNK |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 62 | H_{c}-BoNT/ | IINTSILNLRYESNHLIDLSRYASKINIGS |
| | A1- | KVNFDPIDKNQIQLFNLESSKIEVILKNAI |
| | D1062E (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRETHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 63 | H_{c}-BoNT/ | IINTSILNLRYESNHLIDLSRYASKINIGS |
| | A1- | KVNFDPIDKNQIQLFNLESSKIEVILKNAI |
| | T1063P (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDPHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 64 | H_{c}-BoNT/ | IINTSILNLRYESNHLIDLSRYASKINIGS |
| | A1- | KVNFDPIDKNQIQLFNLESSKIEVILKNAI |
| | H1064R (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTRRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 65 | H_{c}-BoNT/ | IINTSILNLRYESNHLIDLSRYASKINIGS |
| | A1- | KVNFDPIDKNQIQLFNLESSKIEVILKNAI |
| | H1064Q (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTQRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 66 | H_{c}-BoNT/ | IINTSILNLRYESNHLIDLSRYASKINIGS |
| | A1- | KVNFDPIDKNQIQLFNLESSKIEVILKNAI |
| | R1065N (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHNYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 67 | H_{c}-BoNT/ | IINTSILNLRYESNHLIDLSRYASKINIGS |
| | A1- | KVNFDPIDKNQIQLFNLESSKIEVILKNAI |
| | Y1066R (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRRIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 68 | H_{c}-BoNT/ | IINTSILNLRYESNHLIDLSRYASKINIGS |
| | A1- | KVNFDPIDKNQIQLFNLESSKIEVILKNAI |
| | Y1066K (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRKIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 69 | H_{c}-BoNT/ | IINTSILNLRYESNHLIDLSRYASKINIGS |
| | A1- | KVNFDPIDKNQIQLFNLESSKIEVILKNAI |
| | T1145Y (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMYTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 70 | H_{c}-BoNT/ | IINTSILNLRYESNHLIDLSRYASKINIGS |
| | A1- | KVNFDPIDKNQIQLFNLESSKIEVILKNAI |
| | R1156M (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYMGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 71 | H_{c}-BoNT/ | IINTSILNLRYESNHLIDLSRYASKINIGS |
| | A1- | KVNFDPIDKNQIQLFNLESSKIEVILKNAI |
| | R11561 (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYIGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 72 | H_{c}-BoNT/ | IINTSILNLRYESNHLIDLSRYASKINIGS |
| | A1- | KVNFDPIDKNQIQLFNLESSKIEVILKNAI |
| | T1232R (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIR |
| | | NKCKMNLQDNNGNDIGFIGFHQFNNIAKLV |
| | | ASNWYNRQIERSSRTLGCSWEFIPVDDGWG |
| | | ERPL |
| 73 | H_{c}-BoNT/ | IINTSILNLRYESNHLIDLSRYASKINIGS |
| | A1- | KVNFDPIDKNQIQLFNLESSKIEVILKNAI |
| | T1232K (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIK |
| | | NKCKMNLQDNNGNDIGFIGFHQFNNIAKLV |
| | | ASNWYNRQIERSSRTLGCSWEFIPVDDGWG |
| | | ERPL |
| 74 | H_{c}-BoNT/ | IINTSILNLRYESNHLIDLSRYASKINIGS |
| | A1- | KVNFDPIDKNQIQLFNLESSKIEVILKNAI |
| | E1272G (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIGRSSRTLGCSWEFIPVDDGWG |
| | | ERPL |
| 75 | H_{c}-BoNT/ | IINTSILNLRYESNHLIDLSRYASKINIGS |
| | A1- | KVNFDPIDKNQIQLFNLESSKIEVILKNAI |
| | L1278F (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTFGCSWEFIPVDDGWG |
| | | ERPL |
| 76 | H_{c}-BoNT/ | IINTSILNLRYESNHLIDLSRYASKINIGS |
| | A1- | KVNFDPIDKNQIQLFNLESSKIEVILKNAI |
| | L1278Y (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTYGCSWEFIPVDDGWG |
| | | ERPL |
| 77 | H_{c}-BoNT/ | IINTSILNLRYESNHLIDLSRYASKINIGS |
| | A1- | KVNFDPIDKNQIQLFNLESSKIEVILKNAI |
| | L1278W (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTWGCSWEFIPVDDGWG |
| | | ERPL |
| 78 | H_{c}-BoNT/ | IINTSILNLRYESNHLIDLSRYASKINIGS |
| | A1- | KVNFDPIDKNQIQLFNLESSKIEVILKNAI |
| | D1288E (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVEDGWG |
| | | ERPL |
| 79 | H_{c}-BoNT/ | IINTSILNLRYESNHLIDLSRYASKINIGS |
| | A1- | KVNFDPIDKNQIQLFNLESSKIEVILKNAI |
| | D1289Y (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDYGWG |
| | | ERPL |
| 80 | H_{c}-BoNT/ | IINTSILNLRYESNHLIDLSRYASKINIGS |
| | A1- | KVNFDPIDKNQIQLFNLESSKIEVILKNAI |
| | G1292R (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWR |
| | | ERPL |
| 81 | H_{c}-BoNT/ | IINTSILNLRYESNHLIDLSRYASKINIGS |
| | A1- | KVNFDPIDKNQIQLFNLESSKIEVILKNAI |
| | G1292K (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWK |
| | | ERPL |
| 82 | H_{c}-BoNT/ | IINTSILNLRYESNHLIDLSRYASKINIGS |
| | A1- | KVNFDPIDKNQIQLFNLESSKIEVILKNAI |
| | R1294S (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ESPL |
| 83 | H_{c}-BoNT/ | IINTSILNLRYESNHLIDLSRYASKINIGS |
| | A1- | KVNFDPIDKNQIQLFNLESSKIEVILKNAI |
| | R1294T (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ETPL |
| 84 | H_{c}-BoNT/ | IINTSILNLRYESNHLIDLSRYASKINIGS |
| | A1- | KVNFDPIDKNQIQLFNLESSKIEVILKNAI |
| | P1295S (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERSL |
| 85 | H_{c}-BoNT/ | IINTSILNLRYESNHLIDLSRYASKINIGS |
| | A1- | KVNFDPIDKNQIQLFNLESSKIEVILKNAI |
| | P1295T (of | VYNSMYENFSTSFWIRIPKYFNSISLNNEY |
| | full length) | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERTL |
| 86 | H_{c}-BoNT/ | IVNTSILSIVYKKDDLIDLSRYGAKINIGD |
| | A2- | RVYYDSIDKNQIQLINLESSTIEVILKNAI |
| | K915Q (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 87 | H_{c}-BoNT/ | IVNTSILSIVYKKDDLIDLSRYGAKINIGD |
| | A2- | RVYYDSIDKNQIKLINLESSKIEVILKNAI |
| | T923K (of full | VYNSMYENFSTSFWIKIPKYFSKINLNNEY |
| | length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 88 | H_{c}-BoNT/ | IVNTSILSIVYKKDDLIDLSRYGAKINIGD |
| | A2- | RVYYDSIDKNQIKLINLESSTIEVILKNAI |
| | S1090N (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDNQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 89 | H_{c}-BoNT/ | IVNTSILSIVYKKDDLIDLSRYGAKINIGD |
| | A2- | RVYYDSIDKNQIKLINLESSTIEVILKNAI |
| | N1103D (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGDYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 90 | H_{c}-BoNT/ | IVNTSILSIVYKKDDLIDLSRYGAKINIGD |
| | A2- | RVYYDSIDKNQIKLINLESSTIEVILKNAI |
| | F1117Y (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLYDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 91 | H_{c}-BoNT/ | IVNTSILSIVYKKDDLIDLSRYGAKINIGD |
| | A2- | RVYYDSIDKNQIKLINLESSTIEVILKNAI |
| | E1156M (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYMGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 92 | H_{c}-BoNT/ | IVNTSILSIVYKKDDLIDLSRYGAKINIGD |
| | A2- | RVYYDSIDKNQIKLINLESSTIEVILKNAI |
| | E1170K (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNKDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 93 | H_{c}-BoNT/ | IVNTSILSIVYKKDDLIDLSRYGAKINIGD |
| | A2- | RVYYDSIDKNQIKLINLESSTIEVILKNAI |
| | D1227N (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKNDQGIR |
| | | NKCKMNLQDNNGNDIGFIGFHLYDNIAKLV |
| | | ASNWYNRQVGKASRTFGCSWEFIPVDDGWG |
| | | ESSL |
| 94 | H_{c}-BoNT/ | IVNTSILSIVYKKDDLIDLSRYGAKINIGD |
| | A2- | RVYYDSIDKNQIKLINLESSTIEVILKNAI |
| | L1254Q (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHQYDNIAKLV |
| | | ASNWYNRQVGKASRTFGCSWEFIPVDDGWG |
| | | ESSL |
| 95 | H_{c}-BoNT/ | IVNTSILSIVYKKDDLIDLSRYGAKINIGD |
| | A2- | RVYYDSIDKNQIKLINLESSTIEVILKNAI |
| | Y1255F (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLFDNIAKLV |
| | | ASNWYNRQVGKASRTFGCSWEFIPVDDGWG |
| | | ESSL |
| 96 | H_{c}-BoNT/ | IVNTSILSIVYKKDDLIDLSRYGAKINIGD |
| | A2- | RVYYDSIDKNQIKLINLESSTIEVILKNAI |
| | D1256N (of | VYNSMYENFSTSFWIKIPKYFSKINLNNEY |
| | full length) | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYNNIAKLV |
| | | ASNWYNRQVGKASRTFGCSWEFIPVDDGWG |
| | | ESSL |
| 97 | BoNT/ | MPVTINNFNYNDPIDNNNIIMMEPPFARGT |
| | B | GRYYKAFKITDRIWIIPERYTFGYKPEDFN |
| | protease and | KSSGIFNRDVCEYYDPDYLNTNDKKNIFLQ |
| | translocation | TMIKLFNRIKSKPLGEKLLEMIINGIPYLG |
| | domain (AA | DRRVPLEEFNTNIASVTVNKLISNPGEVER |
| | 1-857) | KKGIFANLIIFGPGPVLNENETIDIGIQNH |
| | | FASREGFGGIMQMKFCPEYVSVFNNVQENK |
| | | GASIFNRRGYFSDPALILMHELIHVLHGLY |
| | | GIKVDDLPIVPNEKKFFMQSTDAIQAEELY |
| | | TFGGQDPSIITPSTDKSIYDKVLQNFRGIV |
| | | DRLNKVLVCISDPNININIYKNKFKDKYKF |
| | | VEDSEGKYSIDVESFDKLYKSLMFGFTETN |
| | | IAENYKIKTRASYFSDSLPPVKIKNLLDNE |
| | | IYTIEEGFNISDKDMEKEYRGQNKAINKQA |
| | | YEEISKEHLAVYKIQMCKSVKAPGICIDVD |
| | | NEDLFFIADKNSFSDDLSKNERIEYNTQSN |
| | | YIENDFPINELILDTDLISKIELPSENTES |
| | | LTDFNVDVPVYEKQPAIKKIFTDENTIFQY |
| | | LYSQTFPLDIRDISLTSSFDDALLFSNKVY |
| | | SFFSMDYIKTANKVVEAGLFAGWVKQIVND |
| | | FVIEANKSNTMDKIADISLIVPYIGLALNV |
| | | GNETAKGNFENAFEIAGASILLEFIPELLI |
| | | PVVGAFLLESYIDNKNKIIKTIDNALTKRN |
| | | EKWSDMYGLIVAQWLSTVNTQFYTIKEGMY |
| | | KALNYQAQALEEIIKYRYNIYSEKEKSNIN |
| | | IDFNDINSKLNEGINQAIDNINNFINGCSV |
| | | SYLMKKMIPLAVEKLLDFDNTLKKNLLNYI |
| | | DENKLYLIGSAEYEKSKVNKYLKTIMPFDL |
| | | SIYTNDTILIEMFNKYN |
| 98 | BoNT/ | MPITINNFNYSDPVDNKNILYLDTHLNTLA |
| | C | NEPEKAFRITGNIWVIPDRFSRNSNPNLNK |
| | protease and | PPRVTSPKSGYYDPNYLSTDSDKDTFLKEI |
| | translocation | IKLFKRINSREIGEELIYRLSTDIPFPGNN |
| | domain | NTPINTFDFDVDFNSVDVKTRQGNNWVKTG |
| | (AA 1-870) | SINPSVIITGPRENIIDPETSTFKLTNNTF |
| | | AAQEGFGALSIISISPRFMLTYSNATNDVG |
| | | EGRFSKSEFCMDPILILMHELNHAMHNLYG |
| | | IAIPNDQTISSVTSNIFYSQYNVKLEYAEI |
| | | YAFGGPTIDLIPKSARKYFEEKALDYYRSI |
| | | AKRLNSITTANPSSFNKYIGEYKQKLIRKY |
| | | RFVVESSGEVTVNRNKFVELYNELTQIFTE |
| | | FNYAKIYNVQNRKIYLSNVYTPVTANILDD |
| | | NVYDIQNGFNIPKSNLNVLFMGQNLSRNPA |
| | | LRKVNPENMLYLFTKFCHKAIDGRSLYNKT |
| | | LDCRELLVKNTDLPFIGDISDVKTDIFLRK |
| | | DINEETEVIYYPDNVSVDQVILSKNTSEHG |
| | | QLDLLYPSIDSESEILPGENQVFYDNRTQN |
| | | VDYLNSYYYLESQKLSDNVEDFTFTRSIEE |
| | | ALDNSAKVYTYFPTLANKVNAGVQGGLFLM |
| | | WANDVVEDFTTNILRKDTLDKISDVSAIIP |
| | | YIGPALNISNSVRRGNFTEAFAVTGVTILL |
| | | EAFPEFTIPALGAFVIYSKVQERNEIIKTI |
| | | DNCLEQRIKRWKDSYEWMMGTWLSRIITQF |
| | | NNISYQMYDSLNYQAGAIKAKIDLEYKKYS |
| | | GSDKENIKSQVENLKNSLDVKISEAMNNIN |
| | | KFIRECSVTYLFKNMLPKVIDELNEFDRNT |
| | | KAKLINLIDSHNIILVGEVDKLKAKVNNSF |
| | | QNTIPFNIFSYTNNSLLKDIINEYFNNIND |
| 99 | BoNT/D | MTWPVKDFNYSDPVNDNDILYLRIPQNKLI |
| | protease and | TTPVKAFMITQNIWVIPERFSSDTNPSLSK |
| | translocation | PPRPTSKYQSYYDPSYLSTDEQKDTFLKGI |
| | domain | IKLFKRINERDIGKKLINYLVVGSPFMGDS |
| | (AA 1-862) | STPEDTFDFTRHTTNIAVEKFENGSWKVTN |
| | | IITPSVLIFGPLPNILDYTASLTLQGQQSN |
| | | PSFEGFGTLSILKVAPEFLLTFSDVTSNQS |
| | | SAVLGKSIFCMDPVIALMHELTHSLHQLYG |
| | | INIPSDKRIRPQVSEGFFSQDGPNVQFEEL |
| | | YTFGGLDVEIIPQIERSQLREKALGHYKDI |
| | | AKRLNNINKTIPSSWISNIDKYKKIFSEKY |
| | | NFDKDNTGNFVVNIDKFNSLYSDLTNVMSE |
| | | VVYSSQYNVKNRTHYFSRHYLPVFANILDD |
| | | NIYTIRDGFNLTNKGFNIENSGQNIERNPA |
| | | LQKLSSESVVDLFTKVCLRLTKNSRDDSTC |
| | | IKVKNNRLPYVADKDSISQEIFENKIITDE |
| | | TNVQNYSDNFSLDESILDGQVPINPEIVDP |
| | | LLPNVNMEPLNLPGEEIVFYDDITKYVDYL |
| | | NSYYYLESQKLSNNVENITLTTSVEEALGY |
| | | SNKIYTFLPSLAEKVNKGVQAGLFLNWANE |
| | | VVEDFTTNIMKKDTLDKISDVSVIIPYIGP |
| | | ALNIGNSALRGNFKQAFATAGVAFLLEGFP |
| | | EFTIPALGVFTFYSSIQEREKIIKTIENCL |
| | | EQRVKRWKDSYQWMVSNWLSRITTQFNHIN |
| | | YQMYDSLSYQADAIKAKIDLEYKKYSGSDK |
| | | ENIKSQVENLKNSLDVKISEAMNNINKFIR |
| | | ECSVTYLFKNMLPKVIDELNKFDLRTKTEL |
| | | INLIDSHNIILVGEVDRLKAKVNESFENTM |
| | | PFNIFSYTNNSLLKDIINEYFN |
| 100 | BoNT/ | MPKINSFNYNDPVNDRTILYIKPGGCQEFY |
| | E | KSFNIMKNIWIIPERNVIGTTPQDFHPPTS |
| | protease and | LKNGDSSYYDPNYLQSDEEKDRFLKIVTKI |
| | translocation | FNRINNNLSGGILLEELSKANPYLGNDNTP |
| | domain | DNQFHIGDASAVEIKFSNGSQDILLPNVII |
| | (AA 1-844) | MGAEPDLFETNSSNISLRNNYMPSNHRFGS |
| | | IAIVTFSPEYSFRFNDNCMNEFIQDPALTL |
| | | MHELIHSLHGLYGAKGITTKYTITQKQNPL |
| | | ITNIRGTNIEEFLTFGGTDLNIITSAQSND |
| | | IYTNLLADYKKIASKLSKVQVSNPLLNPYK |
| | | DVFEAKYGLDKDASGIYSVNINKFNDIFKK |
| | | LYSFTEFDLRTKFQVKCRQTYIGQYKYFKL |
| | | SNLLNDSIYNISEGYNINNLKVNFRGQNAN |
| | | LNPRIITPITGRGLVKKIIRFCKNIVSVKG |
| | | IRKSICIEINNGELFFVASENSYNDDNINT |
| | | PKEIDDTVTSNNNYENDLDQVILNFNSESA |
| | | PGLSDEKLNLTIQNDAYIPKYDSNGTSDIE |
| | | QHDVNELNVFFYLDAQKVPEGENNVNLTSS |
| | | IDTALLEQPKIYTFFSSEFINNVNKPVQAA |
| | | LFVSWIQQVLVDFTTEANQKSTVDKIADIS |
| | | IVVPYIGLALNIGNEAQKGNFKDALELLGA |
| | | GILLEFEPELLIPTILVFTIKSFLGSSDNK |
| | | NKVIKAINNALKERDEKWKEVYSFIVSNWM |
| | | TKINTQFNKRKEQMYQALQNQVNAIKTIIE |
| | | SKYNSYTLEEKNELTNKYDIKQIENELNQK |
| | | VSIAMNNIDRFLTESSISYLMKIINEVKIN |
| | | KLREYDENVKTYLLNYIIQHGSILGESQQE |
| | | LNSMVTDTLNNSIPFKLSSYTDDKILISYF |
| | | NKFF |
| 101 | BoNT/ | MPVVINSFNYNDPVNDDTILYMQIPYEEKS |
| | F | KKYYKAFEIMRNVWIIPERNTIGTDPSDFD |
| | protease and | PPASLENGSSAYYDPNYLTTDAEKDRYLKT |
| | translocation | TIKLFKRINSNPAGEVLLQEISYAKPYLGN |
| | domain (1- | EHTPINEFHPVTRTTSVNIKSSTNVKSSII |
| | 863) | LNLLVLGAGPDIFENSSYPVRKLMDSGGVY |
| | | DPSNDGFGSINIVTFSPEYEYTFNDISGGY |
| | | NSSTESFIADPAISLAHELIHALHGLYGAR |
| | | GVTYKETIKVKQAPLMIAEKPIRLEEFLTF |
| | | GGQDLNIITSAMKEKIYNNLLANYEKIATR |
| | | LSRVNSAPPEYDINEYKDYFQWKYGLDKNA |
| | | DGSYTVNENKFNEIYKKLYSFTEIDLANKF |
| | | KVKCRNTYFIKYGFLKVPNLLDDDIYTVSE |
| | | GFNIGNLAVNNRGQNIKLNPKIIDSIPDKG |
| | | LVEKIVKFCKSVIPRKGTKAPPRLCIRVNN |
| | | RELFFVASESSYNENDINTPKEIDDTTNLN |
| | | NNYRNNLDEVILDYNSETIPQISNQTLNTL |
| | | VQDDSYVPRYDSNGTSEIEEHNVVDLNVFF |
| | | YLHAQKVPEGETNISLTSSIDTALSEESQV |
| | | YTFFSSEFINTINKPVHAALFISWINQVIR |
| | | DFTTEATQKSTFDKIADISLVVPYVGLALN |
| | | IGNEVQKENFKEAFELLGAGILLEFVPELL |
| | | IPTILVFTIKSFIGSSENKNKIIKAINNSL |
| | | MERETKWKEIYSWIVSNWLTRINTQFNKRK |
| | | EQMYQALQNQVDAIKTVIEYKYNNYTSDER |
| | | NRLESEYNINNIREELNKKVSLAMENIERF |
| | | ITESSIFYLMKLINEAKVSKLREYDEGVKE |
| | | YLLDYISEHRSILGNSVQELNDLVTSTLNN |
| | | SIPFELSSYTNDKILILYFNKLY |
| 102 | BoNT/ | MPVVINSFNYDDPVNDNTIIYIRPPYYETS |
| | H | NTYFKAFQIMDNVWIIPERYRLGIDPSLFN |
| | protease and | PPVSLKAGSDGYFDPNYLSTNTEKNKYLQI |
| | translocation | MIKLFKRINSKPAGQILLEEIKNAIPYLGN |
| | domain | SYTQEEQFTTNNRTVSFNVKLANGNIVQQM |
| | (AA 1-858) | ANLIIWGPGPDLTTNKTGGIIYSPYQSMEA |
| | | TPYKDGFGSIMTVEFSPEYATAFNDISIAS |
| | | HSPSLFIKDPALILMHELIHVLHGLYGTYI |
| | | TEYKITPNVVQSYMKVTKPITSAEFLTFGG |
| | | RDRNIVPQSIQSQLYNKVLSDYKRIASRLN |
| | | KVNTATALINIDEFKNLYEWKYQFAKDSNG |
| | | VYSVDLNKFEQLYKKIYSFTEFNLAYEFKI |
| | | KTRLGYLAENFGPFYLPNLLDDSIYTEVDG |
| | | FNIGALSINYQGQNIGSDINSIKKLQGQGV |
| | | VSRVVRLCSNSNTKNSLCITVNNRDLFFIA |
| | | SQESYGENTINTYKEIDDTTTLDPSFEDIL |
| | | DKVILNFNEQVIPQMPNRNVSTDIQKDNYI |
| | | PKYDYNRTDIIDSYEVGRNYNTFFYLNAQK |
| | | FSPNESNITLTSSFDTGLLEGSKVYTFFSS |
| | | DFINNINKPVQALLFIEWVKQVIRDFTTEA |
| | | TKTSTVDKLKDISLVVPYIGLALNIGDEIY |
| | | KQHFAEAVELVGAGLLLEFSPEFLIPTLLI |
| | | FTIKGYLTGSIRDKDKIIKTLDNALNVRDQ |
| | | KWKELYRWVVSKWLTTINTQFNKRKEQMYK |
| | | ALKNQATAIKKIIENKYNNYTTDEKSKIDS |
| | | SYNINEIERTLNEKINLAMKNIEQFITESS |
| | | IAYLINIINNETIQKLKSYDDLVRRYLLGY |
| | | IRNHSSILGNSVEELNSKVNNHLDNGIPFE |
| | | LSSYTNDSLLIRYFNKNY |
| 103 | BoNT/ | MKLEINKFNYNDPIDGINVITMRPPRHSDK |
| | X | INKGKGPFKAFQVIKNIWIVPERYNFTNNT |
| | protease and | NDLNIPSEPIMEADAIYNPNYLNTPSEKDE |
| | translocation | FLQGVIKVLERIKSKPEGEKLLELISSSIP |
| | domain | LPLVSNGALTLSDNETIAYQENNNIVSNLQ |
| | (AA 1-889) | ANLVIYGPGPDIANNATYGLYSTPISNGEG |
| | | TLSEVSFSPFYLKPFDESYGNYRSLVNIVN |
| | | KFVKREFAPDPASTLMHELVHVTHNLYGIS |
| | | NRNFYYNFDTGKIETSRQQNSLIFEELLTF |
| | | GGIDSKAISSLIIKKIIETAKNNYTTLISE |
| | | RLNTVTVENDLLKYIKNKIPVQGRLGNFKL |
| | | DTAEFEKKLNTILFVLNESNLAQRFSILVR |
| | | KHYLKERPIDPIYVNILDDNSYSTLEGFNI |
| | | SSQGSNDFQGQLLESSYFEKIESNALRAFI |
| | | KICPRNGLLYNAIYRNSKNYLNNIDLEDKK |
| | | TTSKTNVSYPCSLLNGCIEVENKDLFLISN |
| | | KDSLNDINLSEEKIKPETTVFFKDKLPPQD |
| | | ITLSNYDFTEANSIPSISQQNILERNEELY |
| | | EPIRNSLFEIKTIYVDKLTTFHFLEAQNID |
| | | ESIDSSKIRVELTDSVDEALSNPNKVYSPF |
| | | KNMSNTINSIETGITSTYIFYQ |
| | | WLRSIVKDFSDETGKIDVIDKSSDTLAIVP |
| | | YIGPLLNIGNDIRHGDFVGAIELAGITALL |
| | | EYVPEFTIPILVGLEVIGGELAREQVEAIV |
| | | NNALDKRDQKWAEVYNITKAQWWGTIHLQI |
| | | NTRLAHTYKALSRQANAIKMNMEFQLANYK |
| | | GNIDDKAKIKNAISETEILLNKSVEQAMKN |
| | | TEKFMIKLSNSYLTKEMIPKVQDNLKNFDL |
| | | ETKKTLDKFIKEKEDILGTNLSSSLRRKVS |
| | | IRLNKNIAFDINDIPFSEFDDLINQYK |
| 104 | BoNT/ | MVTINDLHYSDPIDEDNIINMRIPLYDLEV |
| | EN | DDQFINHNVPDLKAFQVFPNVWVVPERYTF |
| | protease and | YSTMKNLDAPANPSRSSYYDPTYLQSDAEK |
| | translocation | EVFLQQMILLFKRINSTQEGQQFLNLLSRS |
| | domain | IPVPYESNGDVAMGTTQVIKQMDDKGNVLK |
| | (1-874) | HRRAHIIIYGPGPDLMAKGSKALTKSRETG |
| | | RGCMAEIYFSPMYHKTYSTKLTNKNSLVDK |
| | | SVQEFVPDPAVTLIHELCHGLHALYGIDLG |
| | | NVGSWEFNSNPNSLFSSWFSSKEAVNFEEV |
| | | MTFGGEDVKVIKSEIDKKIPGILNLIKTTV |
| | | EPIINKITDPHDEMLQCLQSKYPSLKGTLG |
| | | QFFFDDTQLEKDIRDLWMVMNETMFAENLK |
| | | ALTRARYLVPKVENIVQVDILSPNVYTIDK |
| | | GFNHLSKGFKGQSVSQSYFRKISALARGAV |
| | | VRACPNPHFSSQRGLSSCIEILEDDLFIMS |
| | | SKDSFTDTDFSEPSVGPVSYKAKKGADTIL |
| | | DSTLSNYDFSKEINFTSTVPIITVEDPLET |
| | | DEDVPVISEDRTVYVDDYTTFHFLEAQKIG |
| | | KEVVPTQTKVVFTTNMEEALFDSKKVYTVF |
| | | ENTASRINEAGTGIANGMMFYQWLKGIVQD |
| | | FTEEATQKDTFDKISDVTMIVPYLGNILNI |
| | | GNDIRKGDFMGAVELGGVTILLEAIPELTL |
| | | PVLIGLTIIEDELEKEQVSQTVYNVLDKRD |
| | | EKWEEVYGFVKQQWWWMVHTQFETRILHAY |
| | | QALNHQVEAIKANMTYQLANYRGNQEDKEL |
| | | LEKAIDDTLQSLYYAVDQAMHNIKRFLIQS |
| | | SKSYLLNQMLPKTKEQLLAFDQQTLRNVND |
| | | FINKNQGVLGESLAKDLKKKVEKRLTSLPV |
| | | FNLEDLPISEFEDLIHSHEIDIQDSEVLNI |
| | | GVNN |
| 105 | BoNT/ | MPVNIKXFNYNDPINNDDIIMMEPFNDPGP |
| | G | GTYYKAFRIIDRIWIVPERFTYGFQPDQFN |
| | protease and | ASTGVFSKDVYEYYDPTYLKTDAEKDKFLK |
| | translocation | TMIKLFNRINSKPSGQRLLDMIVDAIPYLG |
| | domain | NASTPPDKFAANVANVSINKKIIQPGAEDQ |
| | (AA 1-862) | IKGLMTNLIIFGPGPVLSDNFTDSMIMNGH |
| | | SPISEGFGARMMIRFCPSCLNVFNNVQENK |
| | | DTSIFSRRAYFADPALTLMHELIHVLHGLY |
| | | GIKISNLPITPNTKEFFMQHSDPVQAEELY |
| | | TFGGHDPSVISPSTDMNIYNKALQNFQDIA |
| | | NRLNIVSSAQGSGIDISLYKQIYKNKYDFV |
| | | EDPNGKYSVDKDKFDKLYKALMFGFTETNL |
| | | AGEYGIKTRYSYFSEYLPPIKTEKLLDNTI |
| | | YTQNEGENIASKNLKTEFNGQNKAVNKEAY |
| | | EEISLEHLVIYRIAMCKPVMYKNTGKSEQC |
| | | IIVNNEDLFFIANKDSFSKDLAKAETIAYN |
| | | TQNNTIENNFSIDQLILDNDLSSGIDLPNE |
| | | NTEPFTNFDDIDIPVYIKQSALKKIFVDGD |
| | | SLFEYLHAQTFPSNIENLQLTNSLNDALRN |
| | | NNKVYTFFSTNLVEKANTVVGASLFVNWVK |
| | | GVIDDFTSESTQKSTIDKVSDVSIIIPYIG |
| | | PALNVGNETAKENFKNAFEIGGAAILMEFI |
| | | PELIVPIVGFFTLESYVGNKGHIIMTISNA |
| | | LKKRDQKWTDMYGLIVSQWLSTVNTQFYTI |
| | | KERMYNALNNQSQAIEKIIEDQYNRYSEED |
| | | KMNINIDFNDIDFKLNQSINLAINNIDDFI |
| | | NQCSISYLMNRMIPLAVKKLKDFDDNLKRD |
| | | LLEYIDTNELYLLDEVNILKSKVNRHLKDS |
| | | IPFDLSLYTKDTILIQVENNYI |
| 106 | BoNT/ | MPVTINNFNYNDPIDNNNIIMMEPPFARGT |
| | B-A1 | GRYYKAFKITDRIWIIPERYTFGYKPEDFN |
| | chimera | KSSGIFNRDVCEYYDPDYLNTNDKKNIFLQ |
| | (wildtype A1 | TMIKLFNRIKSKPLGEKLLEMIINGIPYLG |
| | receptor | DRRVPLEEFNTNIASVTVNKLISNPGEVER |
| | binding | KKGIFANLIIFGPGPVLNENETIDIGIQNH |
| | domain) | FASREGFGGIMQMKFCPEYVSVFNNVQENK |
| | | GASIFNRRGYFSDPALILMHELIHVLHGLY |
| | | GIKVDDLPIVPNEKKFFMQSTDAIQAEELY |
| | | TFGGQDPSIITPSTDKSIYDKVLQNFRGIV |
| | | DRLNKVLVCISDPNININIYKNKFKDKYKF |
| | | VEDSEGKYSIDVESFDKLYKSLMFGFTETN |
| | | IAENYKIKTRASYFSDSLPPVKIKNLLDNE |
| | | IYTIEEGFNISDKDMEKEYRGQNKAINKQA |
| | | YEEISKEHLAVYKIQMCKSVKAPGICIDVD |
| | | NEDLFFIADKNSFSDDLSKNERIEYNTQSN |
| | | YIENDFPINELILDTDLISKIELPSENTES |
| | | LTDFNVDVPVYEKQPAIKKIFTDENTIFQY |
| | | LYSQTFPLDIRDISLTSSFDDALLFSNKVY |
| | | SFFSMDYIKTANKVVEAGLFAGWVKQIVND |
| | | FVIEANKSNTMDKIADISLIVPYIGLALNV |
| | | GNETAKGNFENAFEIAGASILLEFIPELLI |
| | | PVVGAFLLESYIDNKNKIIKTIDNALTKRN |
| | | EKWSDMYGLIVAQWLSTVNTQFYTIKEGMY |
| | | KALNYQAQALEEIIKYRYNIYSEKEKSNIN |
| | | IDFNDINSKLNEGINQAIDNINNFINGCSV |
| | | SYLMKKMIPLAVEKLLDFDNTLKKNLLNYI |
| | | DENKLYLIGSAEYEKSKVNKYLKTIMPFDL |
| | | SIYTNDTILIEMFNKYNIINTSILNLRYES |
| | | NHLIDLSRYASKINIGSKVNFDPIDKNQIQ |
| | | LFNLESSKIEVILKNAIVYNSMYENFSTSF |
| | | WIRIPKYFNSISLNNEYTIINCMENNSGWK |
| | | VSLNYGEIIWTLQDTQEIKQRVVFKYSQMI |
| | | NISDYINRWIFVTITNNRLNNSKIYINGRL |
| | | IDQKPISNLGNIHASNNIMFKLDGCRDTHR |
| | | YIWIKYFNLFDKELNEKEIKDLYDNQSNSG |
| | | ILKDFWGDYLQYDKPYYMLNLYDPNKYVDV |
| | | NNVGIRGYMYLKGPRGSVMTTNIYLNSSLY |
| | | RGTKFIIKKYASGNKDNIVRNNDRVYINVV |
| | | VKNKEYRLATNASQAGVEKILSALEIPDVG |
| | | NLSQVVVMKSKNDQGITNKCKMNLQDNNGN |
| | | DIGFIGFHQFNNIAKLVASNWYNRQIERSS |
| | | RTLGCSWEFIPVDDGWGERPL |
| 107 | BoNT/ | MPITINNFNYSDPVDNKNILYLDTHLNTLA |
| | C-A1 | NEPEKAFRITGNIWVIPDRFSRNSNPNLNK |
| | chimera | PPRVTSPKSGYYDPNYLSTDSDKDTFLKEI |
| | (wildtype A1 | IKLFKRINSREIGEELIYRLSTDIPFPGNN |
| | receptor | NTPINTFDFDVDFNSVDVKTRQGNNWVKTG |
| | binding | SINPSVIITGPRENIIDPETSTFKLTNNTF |
| | domain) | AAQEGFGALSIISISPRFMLTYSNATNDVG |
| | | EGRFSKSEFCMDPILILMHELNHAMHNLYG |
| | | IAIPNDQTISSVTSNIFYSQYNVKLEYAEI |
| | | YAFGGPTIDLIPKSARKYFEEKALDYYRSI |
| | | AKRLNSITTANPSSFNKYIGEYKQKLIRKY |
| | | RFVVESSGEVTVNRNKFVELYNELTQIFTE |
| | | FNYAKIYNVQNRKIYLSNVYTPVTANILDD |
| | | NVYDIQNGFNIPKSNLNVLFMGQNLSRNPA |
| | | LRKVNPENMLYLFTKFCHKAIDGRSLYNKT |
| | | LDCRELLVKNTDLPFIGDISDVKTDIFLRK |
| | | DINEETEVIYYPDNVSVDQVILSKNTSEHG |
| | | QLDLLYPSIDSESEILPGENQVFYDNRTQN |
| | | VDYLNSYYYLESQKLSDNVEDFTFTRSIEE |
| | | ALDNSAKVYTYFPTLANKVNAGVQGGLFLM |
| | | WANDVVEDFTTNILRKDTLDKISDVSAIIP |
| | | YIGPALNISNSVRRGNFTEAFAVTGVTILL |
| | | EAFPEFTIPALGAFVIYSKVQERNEIIKTI |
| | | DNCLEQRIKRWKDSYEWMMGTWLSRIITQF |
| | | NNISYQMYDSLNYQAGAIKAKIDLEYKKYS |
| | | GSDKENIKSQVENLKNSLDVKISEAMNNIN |
| | | KFIRECSVTYLFKNMLPKVIDELNEFDRNT |
| | | KAKLINLIDSHNIILVGEVDKLKAKVNNSF |
| | | QNTIPFNIFSYTNNSLLKDIINEYFNNIND |
| | | IINTSILNLRYESNHLIDLSRYASKINIGS |
| | | KVNFDPIDKNQIQLFNLESSKIEVILKNAI |
| | | VYNSMYENFSTSFWIRIPKYFNSISLNNEY |
| | | TIINCMENNSGWKVSLNYGEIIWTLQDTQE |
| | | IKQRVVFKYSQMINISDYINRWIFVTITNN |
| | | RLNNSKIYINGRLIDQKPISNLGNIHASNN |
| | | IMFKLDGCRDTHRYIWIKYFNLFDKELNEK |
| | | EIKDLYDNQSNSGILKDFWGDYLQYDKPYY |
| | | MLNLYDPNKYVDVNNVGIRGYMYLKGPRGS |
| | | VMTTNIYLNSSLYRGTKFIIKKYASGNKDN |
| | | IVRNNDRVYINVVVKNKEYRLATNASQAGV |
| | | EKILSALEIPDVGNLSQVVVMKSKNDQGIT |
| | | NKCKMNLQDNNGNDIGFIGFHQFNNIAKLV |
| | | ASNWYNRQIERSSRTLGCSWEFIPVDDGWG |
| | | ERPL |
| 108 | | MTWPVKDFNYSDPVNDNDILYLRIPQNKLI |
| | BoNT/ | TTPVKAFMITQNIWVIPERFSSDTNPSLSK |
| | D-A1 | PPRPTSKYQSYYDPSYLSTDEQKDTFLKGI |
| | chimera | IKLFKRINERDIGKKLINYLVVGSPFMGDS |
| | (wildtype A1 | STPEDTFDFTRHTTNIAVEKFENGSWKVTN |
| | receptor | IITPSVLIFGPLPNILDYTASLTLQGQQSN |
| | binding | PSFEGFGTLSILKVAPEFLLTFSDVTSNQS |
| | domain) | SAVLGKSIFCMDPVIALMHELTHSLHQLYG |
| | | INIPSDKRIRPQVSEGFFSQDGPNVQFEEL |
| | | YTFGGLDVEIIPQIERSQLREKALGHYKDI |
| | | AKRLNNINKTIPSSWISNIDKYKKIFSEKY |
| | | NFDKDNTGNFVVNIDKFNSLYSDLTNVMSE |
| | | VVYSSQYNVKNRTHYFSRHYLPVFANILDD |
| | | NIYTIRDGFNLTNKGFNIENSGQNIERNPA |
| | | LQKLSSESVVDLFTKVCLRLTKNSRDDSTC |
| | | IKVKNNRLPYVADKDSISQEIFENKIITDE |
| | | TNVQNYSDNFSLDESILDGQVPINPEIVDP |
| | | LLPNVNMEPLNLPGEEIVFYDDITKYVDYL |
| | | NSYYYLESQKLSNNVENITLTTSVEEALGY |
| | | SNKIYTFLPSLAEKVNKGVQAGLFLNWANE |
| | | VVEDFTTNIMKKDTLDKISDVSVIIPYIGP |
| | | ALNIGNSALRGNFKQAFATAGVAFLLEGFP |
| | | EFTIPALGVFTFYSSIQEREKIIKTIENCL |
| | | EQRVKRWKDSYQWMVSNWLSRITTQFNHIN |
| | | YQMYDSLSYQADAIKAKIDLEYKKYSGSDK |
| | | ENIKSQVENLKNSLDVKISEAMNNINKFIR |
| | | ECSVTYLFKNMLPKVIDELNKFDLRTKTEL |
| | | INLIDSHNIILVGEVDRLKAKVNESFENTM |
| | | PFNIFSYTNNSLLKDIINEYFNIINTSILN |
| | | LRYESNHLIDLSRYASKINIGSKVNFDPID |
| | | KNQIQLFNLESSKIEVILKNAIVYNSMYEN |
| | | FSTSFWIRIPKYFNSISLNNEYTIINCMEN |
| | | NSGWKVSLNYGEIIWTLQDTQEIKQRVVFK |
| | | YSQMINISDYINRWIFVTITNNRLNNSKIY |
| | | INGRLIDQKPISNLGNIHASNNIMFKLDGC |
| | | RDTHRYIWIKYFNLFDKELNEKEIKDLYDN |
| | | QSNSGILKDFWGDYLQYDKPYYMLNLYDPN |
| | | KYVDVNNVGIRGYMYLKGPRGSVMTTNIYL |
| | | NSSLYRGTKFIIKKYASGNKDNIVRNNDRV |
| | | YINVVVKNKEYRLATNASQAGVEKILSALE |
| | | IPDVGNLSQVVVMKSKNDQGITNKCKMNLQ |
| | | DNNGNDIGFIGFHQFNNIAKLVASNWYNRQ |
| | | IERSSRTLGCSWEFIPVDDGWGERPL |
| 109 | BoNT/ | MPKINSFNYNDPVNDRTILYIKPGGCQEFY |
| | E-A1 | KSFNIMKNIWIIPERNVIGTTPQDFHPPTS |
| | chimera | LKNGDSSYYDPNYLQSDEEKDRFLKIVTKI |
| | (wildtype A1 | FNRINNNLSGGILLEELSKANPYLGNDNTP |
| | receptor | DNQFHIGDASAVEIKFSNGSQDILLPNVII |
| | binding | MGAEPDLFETNSSNISLRNNYMPSNHRFGS |
| | domain) | IAIVTFSPEYSFRFNDNCMNEFIQDPALTL |
| | | MHELIHSLHGLYGAKGITTKYTITQKQNPL |
| | | ITNIRGTNIEEFLTFGGTDLNIITSAQSND |
| | | IYTNLLADYKKIASKLSKVQVSNPLLNPYK |
| | | DVFEAKYGLDKDASGIYSVNINKENDIFKK |
| | | LYSFTEFDLRTKFQVKCRQTYIGQYKYFKL |
| | | SNLLNDSIYNISEGYNINNLKVNFRGQNAN |
| | | LNPRIITPITGRGLVKKIIRFCKNIVSVKG |
| | | IRKSICIEINNGELFFVASENSYNDDNINT |
| | | PKEIDDTVTSNNNYENDLDQVILNFNSESA |
| | | PGLSDEKLNLTIQNDAYIPKYDSNGTSDIE |
| | | QHDVNELNVFFYLDAQKVPEGENNVNLTSS |
| | | IDTALLEQPKIYTFFSSEFINNVNKPVQAA |
| | | LFVSWIQQVLVDFTTEANQKSTVDKIADIS |
| | | IVVPYIGLALNIGNEAQKGNFKDALELLGA |
| | | GILLEFEPELLIPTILVFTIKSFLGSSDNK |
| | | NKVIKAINNALKERDEKWKEVYSFIVSNWM |
| | | TKINTQFNKRKEQMYQALQNQVNAIKTIIE |
| | | SKYNSYTLEEKNELTNKYDIKQIENELNQK |
| | | VSIAMNNIDRFLTESSISYLMKIINEVKIN |
| | | KLREYDENVKTYLLNYIIQHGSILGESQQE |
| | | LNSMVTDTLNNSIPFKLSSYTDDKILISYF |
| | | NKFFIINTSILNLRYESNHLIDLSRYASKI |
| | | NIGSKVNFDPIDKNQIQLFNLESSKIEVIL |
| | | KNAIVYNSMYENFSTSFWIRIPKYFNSISL |
| | | NNEYTIINCMENNSGWKVSLNYGEIIWTLQ |
| | | DTQEIKQRVVFKYSQMINISDYINRWIFVT |
| | | ITNNRLNNSKIYINGRLIDQKPISNLGNIH |
| | | ASNNIMFKLDGCRDTHRYIWIKYFNLFDKE |
| | | LNEKEIKDLYDNQSNSGILKDFWGDYLQYD |
| | | KPYYMLNLYDPNKYVDVNNVGIRGYMYLKG |
| | | PRGSVMTTNIYLNSSLYRGTKFIIKKYASG |
| | | NKDNIVRNNDRVYINVVVKNKEYRLATNAS |
| | | QAGVEKILSALEIPDVGNLSQVVVMKSKND |
| | | QGITNKCKMNLQDNNGNDIGFIGFHQFNNI |
| | | AKLVASNWYNRQIERSSRTLGCSWEFIPVD |
| | | DGWGERPL |
| 110 | BoNT/ | MPVVINSFNYNDPVNDDTILYMQIPYEEKS |
| | F-A1 | KKYYKAFEIMRNVWIIPERNTIGTDPSDFD |
| | chimera | PPASLENGSSAYYDPNYLTTDAEKDRYLKT |
| | (wildtype A1 | TIKLFKRINSNPAGEVLLQEISYAKPYLGN |
| | receptor | EHTPINEFHPVTRTTSVNIKSSTNVKSSII |
| | binding | LNLLVLGAGPDIFENSSYPVRKLMDSGGVY |
| | domain) | DPSNDGFGSINIVTFSPEYEYTFNDISGGY |
| | | NSSTESFIADPAISLAHELIHALHGLYGAR |
| | | GVTYKETIKVKQAPLMIAEKPIRLEEFLTF |
| | | GGQDLNIITSAMKEKIYNNLLANYEKIATR |
| | | LSRVNSAPPEYDINEYKDYFQWKYGLDKNA |
| | | DGSYTVNENKFNEIYKKLYSFTEIDLANKF |
| | | KVKCRNTYFIKYGFLKVPNLLDDDIYTVSE |
| | | GFNIGNLAVNNRGQNIKLNPKIIDSIPDKG |
| | | LVEKIVKFCKSVIPRKGTKAPPRLCIRVNN |
| | | RELFFVASESSYNENDINTPKEIDDTTNLN |
| | | NNYRNNLDEVILDYNSETIPQISNQTLNTL |
| | | VQDDSYVPRYDSNGTSEIEEHNVVDLNVFF |
| | | YLHAQKVPEGETNISLTSSIDTALSEESQV |
| | | YTFFSSEFINTINKPVHAALFISWINQVIR |
| | | DFTTEATQKSTFDKIADISLVVPYVGLALN |
| | | IGNEVQKENFKEAFELLGAGILLEFVPELL |
| | | IPTILVFTIKSFIGSSENKNKIIKAINNSL |
| | | MERETKWKEIYSWIVSNWLTRINTQFNKRK |
| | | EQMYQALQNQVDAIKTVIEYKYNNYTSDER |
| | | NRLESEYNINNIREELNKKVSLAMENIERF |
| | | ITESSIFYLMKLINEAKVSKLREYDEGVKE |
| | | YLLDYISEHRSILGNSVQELNDLVTSTLNN |
| | | SIPFELSSYTNDKILILYFNKLYIINTSIL |
| | | NLRYESNHLIDLSRYASKINIGSKVNFDPI |
| | | DKNQIQLFNLESSKIEVILKNAIVYNSMYE |
| | | NFSTSFWIRIPKYFNSISLNNEYTIINCME |
| | | NNSGWKVSLNYGEIIWTLQDTQEIKQRVVF |
| | | KYSQMINISDYINRWIFVTITNNRLNNSKI |
| | | YINGRLIDQKPISNLGNIHASNNIMFKLDG |
| | | CRDTHRYIWIKYFNLFDKELNEKEIKDLYD |
| | | NQSNSGILKDFWGDYLQYDKPYYMLNLYDP |
| | | NKYVDVNNVGIRGYMYLKGPRGSVMTTNIY |
| | | LNSSLYRGTKFIIKKYASGNKDNIVRNNDR |
| | | VYINVVVKNKEYRLATNASQAGVEKILSAL |
| | | EIPDVGNLSQVVVMKSKNDQGITNKCKMNL |
| | | QDNNGNDIGFIGFHQFNNIAKLVASNWYNR |
| | | QIERSSRTLGCSWEFIPVDDGWGERPL |
| 111 | BoNT/ | MPVNIKXFNYNDPINNDDIIMMEPFNDPGP |
| | G-A1 | GTYYKAFRIIDRIWIVPERFTYGFQPDQFN |
| | chimera | ASTGVFSKDVYEYYDPTYLKTDAEKDKFLK |
| | (wildtype A1 | TMIKLFNRINSKPSGQRLLDMIVDAIPYLG |
| | receptor | NASTPPDKFAANVANVSINKKIIQPGAEDQ |
| | binding | IKGLMTNLIIFGPGPVLSDNFTDSMIMNGH |
| | domain) | SPISEGFGARMMIRFCPSCLNVFNNVQENK |
| | | DTSIFSRRAYFADPALTLMHELIHVLHGLY |
| | | GIKISNLPITPNTKEFFMQHSDPVQAEELY |
| | | TFGGHDPSVISPSTDMNIYNKALQNFQDIA |
| | | NRLNIVSSAQGSGIDISLYKQIYKNKYDFV |
| | | EDPNGKYSVDKDKFDKLYKALMFGFTETNL |
| | | AGEYGIKTRYSYFSEYLPPIKTEKLLDNTI |
| | | YTQNEGENIASKNLKTEFNGQNKAVNKEAY |
| | | EEISLEHLVIYRIAMCKPVMYKNTGKSEQC |
| | | IIVNNEDLFFIANKDSFSKDLAKAETIAYN |
| | | TQNNTIENNFSIDQLILDNDLSSGIDLPNE |
| | | NTEPFTNFDDIDIPVYIKQSALKKIFVDGD |
| | | SLFEYLHAQTFPSNIENLQLTNSLNDALRN |
| | | NNKVYTFFSTNLVEKANTVVGASLFVNWVK |
| | | GVIDDFTSESTQKSTIDKVSDVSIIIPYIG |
| | | PALNVGNETAKENFKNAFEIGGAAILMEFI |
| | | PELIVPIVGFFTLESYVGNKGHIIMTISNA |
| | | LKKRDQKWTDMYGLIVSQWLSTVNTQFYTI |
| | | KERMYNALNNQSQAIEKIIEDQYNRYSEED |
| | | KMNINIDFNDIDFKLNQSINLAINNIDDFI |
| | | NQCSISYLMNRMIPLAVKKLKDFDDNLKRD |
| | | LLEYIDTNELYLLDEVNILKSKVNRHLKDS |
| | | IPFDLSLYTKDTILIQVFNNYIIINTSILN |
| | | LRYESNHLIDLSRYASKINIGSKVNFDPID |
| | | KNQIQLFNLESSKIEVILKNAIVYNSMYEN |
| | | FSTSFWIRIPKYFNSISLNNEYTIINCMEN |
| | | NSGWKVSLNYGEIIWTLQDTQEIKQRVVFK |
| | | YSQMINISDYINRWIFVTITNNRLNNSKIY |
| | | INGRLIDQKPISNLGNIHASNNIMFKLDGC |
| | | RDTHRYIWIKYFNLFDKELNEKEIKDLYDN |
| | | QSNSGILKDFWGDYLQYDKPYYMLNLYDPN |
| | | KYVDVNNVGIRGYMYLKGPRGSVMTTNIYL |
| | | NSSLYRGTKFIIKKYASGNKDNIVRNNDRV |
| | | YINVVVKNKEYRLATNASQAGVEKILSALE |
| | | IPDVGNLSQVVVMKSKNDQGITNKCKMNLQ |
| | | DNNGNDIGFIGFHQFNNIAKLVASNWYNRQ |
| | | IERSSRTLGCSWEFIPVDDGWGERPL |
| 112 | BoNT/ | MPVVINSFNYDDPVNDNTIIYIRPPYYETS |
| | H-A1 | NTYFKAFQIMDNVWIIPERYRLGIDPSLFN |
| | chimera | PPVSLKAGSDGYFDPNYLSTNTEKNKYLQI |
| | (wildtype A1 | MIKLFKRINSKPAGQILLEEIKNAIPYLGN |
| | receptor | SYTQEEQFTTNNRTVSFNVKLANGNIVQQM |
| | binding | ANLIIWGPGPDLTTNKTGGIIYSPYQSMEA |
| | domain) | TPYKDGFGSIMTVEFSPEYATAFNDISIAS |
| | | HSPSLFIKDPALILMHELIHVLHGLYGTYI |
| | | TEYKITPNVVQSYMKVTKPITSAEFLTFGG |
| | | RDRNIVPQSIQSQLYNKVLSDYKRIASRLN |
| | | KVNTATALINIDEFKNLYEWKYQFAKDSNG |
| | | VYSVDLNKFEQLYKKIYSFTEFNLAYEFKI |
| | | KTRLGYLAENFGPFYLPNLLDDSIYTEVDG |
| | | FNIGALSINYQGQNIGSDINSIKKLQGQGV |
| | | VSRVVRLCSNSNTKNSLCITVNNRDLFFIA |
| | | SQESYGENTINTYKEIDDTTTLDPSFEDIL |
| | | DKVILNFNEQVIPQMPNRNVSTDIQKDNYI |
| | | PKYDYNRTDIIDSYEVGRNYNTFFYLNAQK |
| | | FSPNESNITLTSSFDTGLLEGSKVYTFFSS |
| | | DFINNINKPVQALLFIEWVKQVIRDFTTEA |
| | | TKTSTVDKLKDISLVVPYIGLALNIGDEIY |
| | | KQHFAEAVELVGAGLLLEFSPEFLIPTLLI |
| | | FTIKGYLTGSIRDKDKIIKTLDNALNVRDQ |
| | | KWKELYRWVVSKWLTTINTQFNKRKEQMYK |
| | | ALKNQATAIKKIIENKYNNYTTDEKSKIDS |
| | | SYNINEIERTLNEKINLAMKNIEQFITESS |
| | | IAYLINIINNETIQKLKSYDDLVRRYLLGY |
| | | IRNHSSILGNSVEELNSKVNNHLDNGIPFE |
| | | LSSYTNDSLLIRYFNKNYIINTSILNLRYE |
| | | SNHLIDLSRYASKINIGSKVNFDPIDKNQI |
| | | QLFNLESSKIEVILKNAIVYNSMYENFSTS |
| | | FWIRIPKYFNSISLNNEYTIINCMENNSGW |
| | | KVSLNYGEIIWTLQDTQEIKQRVVFKYSQM |
| | | INISDYINRWIFVTITNNRLNNSKIYINGR |
| | | LIDQKPISNLGNIHASNNIMFKLDGCRDTH |
| | | RYIWIKYFNLFDKELNEKEIKDLYDNQSNS |
| | | GILKDFWGDYLQYDKPYYMLNLYDPNKYVD |
| | | VNNVGIRGYMYLKGPRGSVMTTNIYLNSSL |
| | | YRGTKFIIKKYASGNKDNIVRNNDRVYINV |
| | | VVKNKEYRLATNASQAGVEKILSALEIPDV |
| | | GNLSQVVVMKSKNDQGITNKCKMNLQDNNG |
| | | NDIGFIGFHQFNNIAKLVASNWYNRQIERS |
| | | SRTLGCSWEFIPVDDGWGERPL |
| 113 | BoNT/ | MKLEINKFNYNDPIDGINVITMRPPRHSDK |
| | X-A1 | INKGKGPFKAFQVIKNIWIVPERYNFTNNT |
| | chimera | NDLNIPSEPIMEADAIYNPNYLNTPSEKDE |
| | (wildtype A1 | FLQGVIKVLERIKSKPEGEKLLELISSSIP |
| | receptor | LPLVSNGALTLSDNETIAYQENNNIVSNLQ |
| | binding | ANLVIYGPGPDIANNATYGLYSTPISNGEG |
| | domain) | TLSEVSFSPFYLKPFDESYGNYRSLVNIVN |
| | | KFVKREFAPDPASTLMHELVHVTHNLYGIS |
| | | NRNFYYNFDTGKIETSRQQNSLIFEELLTF |
| | | GGIDSKAISSLIIKKIIETAKNNYTTLISE |
| | | RLNTVTVENDLLKYIKNKIPVQGRLGNFKL |
| | | DTAEFEKKLNTILFVLNESNLAQRFSILVR |
| | | KHYLKERPIDPIYVNILDDNSYSTLEGFNI |
| | | SSQGSNDFQGQLLESSYFEKIESNALRAFI |
| | | KICPRNGLLYNAIYRNSKNYLNNIDLEDKK |
| | | TTSKTNVSYPCSLLNGCIEVENKDLFLISN |
| | | KDSLNDINLSEEKIKPETTVFFKDKLPPQD |
| | | ITLSNYDFTEANSIPSISQQNILERNEELY |
| | | EPIRNSLFEIKTIYVDKLTTFHFLEAQNID |
| | | ESIDSSKIRVELTDSVDEALSNPNKVYSPF |
| | | KNMSNTINSIETGITSTYIFYQWLRSIVKD |
| | | FSDETGKIDVIDKSSDTLAIVPYIGPLLNI |
| | | GNDIRHGDFVGAIELAGITALLEYVPEFTI |
| | | PILVGLEVIGGELAREQVEAIVNNALDKRD |
| | | QKWAEVYNITKAQWWGTIHLQINTRLAHTY |
| | | KALSRQANAIKMNMEFQLANYKGNIDDKAK |
| | | IKNAISETEILLNKSVEQAMKNTEKFMIKL |
| | | SNSYLTKEMIPKVQDNLKNFDLETKKTLDK |
| | | FIKEKEDILGTNLSSSLRRKVSIRLNKNIA |
| | | FDINDIPFSEFDDLINQYKIINTSILNLRY |
| | | ESNHLIDLSRYASKINIGSKVNFDPIDKNQ |
| | | IQLFNLESSKIEVILKNAIVYNSMYENFST |
| | | SFWIRIPKYFNSISLNNEYTIINCMENNSG |
| | | WKVSLNYGEIIWTLQDTQEIKQRVVFKYSQ |
| | | MINISDYINRWIFVTITNNRLNNSKIYING |
| | | RLIDQKPISNLGNIHASNNIMFKLDGCRDT |
| | | HRYIWIKYFNLFDKELNEKEIKDLYDNQSN |
| | | SGILKDFWGDYLQYDKPYYMLNLYDPNKYV |
| | | DVNNVGIRGYMYLKGPRGSVMTTNIYLNSS |
| | | LYRGTKFIIKKYASGNKDNIVRNNDRVYIN |
| | | VVVKNKEYRLATNASQAGVEKILSALEIPD |
| | | VGNLSQVVVMKSKNDQGITNKCKMNLQDNN |
| | | GNDIGFIGFHQFNNIAKLVASNWYNRQIER |
| | | SSRTLGCSWEFIPVDDGWGERPL |
| 114 | BoNT/ | MVTINDLHYSDPIDEDNIINMRIPLYDLEV |
| | EN-A1 | DDQFINHNVPDLKAFQVFPNVWVVPERYTF |
| | chimera | YSTMKNLDAPANPSRSSYYDPTYLQSDAEK |
| | (wildtype A1 | EVFLQQMILLFKRINSTQEGQQFLNLLSRS |
| | receptor | IPVPYESNGDVAMGTTQVIKQMDDKGNVLK |
| | binding | HRRAHIIIYGPGPDLMAKGSKALTKSRETG |
| | domain) | RGCMAEIYFSPMYHKTYSTKLTNKNSLVDK |
| | | SVQEFVPDPAVTLIHELCHGLHALYGIDLG |
| | | NVGSWEFNSNPNSLFSSWFSSKEAVNFEEV |
| | | MTFGGEDVKVIKSEIDKKIPGILNLIKTTV |
| | | EPIINKITDPHDEMLQCLQSKYPSLKGTLG |
| | | QFFFDDTQLEKDIRDLWMVMNETMFAENLK |
| | | ALTRARYLVPKVENIVQVDILSPNVYTIDK |
| | | GFNHLSKGFKGQSVSQSYFRKISALARGAV |
| | | VRACPNPHFSSQRGLSSCIEILEDDLFIMS |
| | | SKDSFTDTDFSEPSVGPVSYKAKKGADTIL |
| | | DSTLSNYDFSKEINFTSTVPIITVEDPLET |
| | | DEDVPVISEDRTVYVDDYTTFHFLEAQKIG |
| | | KEVVPTQTKVVFTTNMEEALFDSKKVYTVF |
| | | ENTASRINEAGTGIANGMMFYQWLKGIVQD |
| | | FTEEATQKDTFDKISDVTMIVPYLGNILNI |
| | | GNDIRKGDFMGAVELGGVTILLEAIPELTL |
| | | PVLIGLTIIEDELEKEQVSQTVYNVLDKRD |
| | | EKWEEVYGFVKQQWWWMVHTQFETRILHAY |
| | | QALNHQVEAIKANMTYQLANYRGNQEDKEL |
| | | LEKAIDDTLQSLYYAVDQAMHNIKRFLIQS |
| | | SKSYLLNQMLPKTKEQLLAFDQQTLRNVND |
| | | FINKNQGVLGESLAKDLKKKVEKRLTSLPV |
| | | FNLEDLPISEFEDLIHSHEIDIQDSEVLNI |
| | | GVNNIINTSILNLRYESNHLIDLSRYASKI |
| | | NIGSKVNFDPIDKNQIQLFNLESSKIEVIL |
| | | KNAIVYNSMYENFSTSFWIRIPKYFNSISL |
| | | NNEYTIINCMENNSGWKVSLNYGEIIWTLQ |
| | | DTQEIKQRVVFKYSQMINISDYINRWIFVT |
| | | ITNNRLNNSKIYINGRLIDQKPISNLGNIH |
| | | ASNNIMFKLDGCRDTHRYIWIKYFNLFDKE |
| | | LNEKEIKDLYDNQSNSGILKDFWGDYLQYD |
| | | KPYYMLNLYDPNKYVDVNNVGIRGYMYLKG |
| | | PRGSVMTTNIYLNSSLYRGTKFIIKKYASG |
| | | NKDNIVRNNDRVYINVVVKNKEYRLATNAS |
| | | QAGVEKILSALEIPDVGNLSQVVVMKSKND |
| | | QGITNKCKMNLQDNNGNDIGFIGFHQFNNI |
| | | AKLVASNWYNRQIERSSRTLGCSWEFIPVD |
| | | DGWGERPL |
| 115 | BoNT/ | MPVTINNFNYNDPIDNNNIIMMEPPFARGT |
| | B-A2 | GRYYKAFKITDRIWIIPERYTFGYKPEDFN |
| | chimera | KSSGIFNRDVCEYYDPDYLNTNDKKNIFLQ |
| | (wildtype A2 | TMIKLFNRIKSKPLGEKLLEMIINGIPYLG |
| | receptor | DRRVPLEEFNTNIASVTVNKLISNPGEVER |
| | binding | KKGIFANLIIFGPGPVLNENETIDIGIQNH |
| | domain) | FASREGFGGIMQMKFCPEYVSVFNNVQENK |
| | | GASIFNRRGYFSDPALILMHELIHVLHGLY |
| | | GIKVDDLPIVPNEKKFFMQSTDAIQAEELY |
| | | TFGGQDPSIITPSTDKSIYDKVLQNFRGIV |
| | | DRLNKVLVCISDPNININIYKNKFKDKYKF |
| | | VEDSEGKYSIDVESFDKLYKSLMFGFTETN |
| | | IAENYKIKTRASYFSDSLPPVKIKNLLDNE |
| | | IYTIEEGFNISDKDMEKEYRGQNKAINKQA |
| | | YEEISKEHLAVYKIQMCKSVKAPGICIDVD |
| | | NEDLFFIADKNSFSDDLSKNERIEYNTQSN |
| | | YIENDFPINELILDTDLISKIELPSENTES |
| | | LTDFNVDVPVYEKQPAIKKIFTDENTIFQY |
| | | LYSQTFPLDIRDISLTSSFDDALLFSNKVY |
| | | SFFSMDYIKTANKVVEAGLFAGWVKQIVND |
| | | FVIEANKSNTMDKIADISLIVPYIGLALNV |
| | | GNETAKGNFENAFEIAGASILLEFIPELLI |
| | | PVVGAFLLESYIDNKNKIIKTIDNALTKRN |
| | | EKWSDMYGLIVAQWLSTVNTQFYTIKEGMY |
| | | KALNYQAQALEEIIKYRYNIYSEKEKSNIN |
| | | IDFNDINSKLNEGINQAIDNINNFINGCSV |
| | | SYLMKKMIPLAVEKLLDFDNTLKKNLLNYI |
| | | DENKLYLIGSAEYEKSKVNKYLKTIMPFDL |
| | | SIYTNDTILIEMFNKYNIVNTSILSIVYKK |
| | | DDLIDLSRYGAKINIGDRVYYDSIDKNQIK |
| | | LINLESSTIEVILKNAIVYNSMYENFSTSF |
| | | WIKIPKYFSKINLNNEYTIINCIENNSGWK |
| | | VSLNYGEIIWTLQDNKQNIQRVVFKYSQMV |
| | | NISDYINRWIFVTITNNRLTKSKIYINGRL |
| | | IDQKPISNLGNIHASNKIMFKLDGCRDPRR |
| | | YIMIKYFNLFDKELNEKEIKDLYDSQSNSG |
| | | ILKDFWGNYLQYDKPYYMLNLFDPNKYVDV |
| | | NNIGIRGYMYLKGPRGSVVTTNIYLNSTLY |
| | | EGTKFIIKKYASGNEDNIVRNNDRVYINVV |
| | | VKNKEYRLATNASQAGVEKILSALEIPDVG |
| | | NLSQVVVMKSKDDQGIRNKCKMNLQDNNGN |
| | | DIGFIGFHLYDNIAKLVASNWYNRQVGKAS |
| | | RTFGCSWEFIPVDDGWGESSL |
| 116 | BoNT/ | MPITINNFNYSDPVDNKNILYLDTHLNTLA |
| | C-A2 | NEPEKAFRITGNIWVIPDRFSRNSNPNLNK |
| | chimera | PPRVTSPKSGYYDPNYLSTDSDKDTFLKEI |
| | (wildtype A2 | IKLFKRINSREIGEELIYRLSTDIPFPGNN |
| | receptor | NTPINTFDFDVDFNSVDVKTRQGNNWVKTG |
| | binding | SINPSVIITGPRENIIDPETSTFKLTNNTF |
| | domain) | AAQEGFGALSIISISPRFMLTYSNATNDVG |
| | | EGRFSKSEFCMDPILILMHELNHAMHNLYG |
| | | IAIPNDQTISSVTSNIFYSQYNVKLEYAEI |
| | | YAFGGPTIDLIPKSARKYFEEKALDYYRSI |
| | | AKRLNSITTANPSSFNKYIGEYKQKLIRKY |
| | | RFVVESSGEVTVNRNKFVELYNELTQIFTE |
| | | FNYAKIYNVQNRKIYLSNVYTPVTANILDD |
| | | NVYDIQNGFNIPKSNLNVLFMGQNLSRNPA |
| | | LRKVNPENMLYLFTKFCHKAIDGRSLYNKT |
| | | LDCRELLVKNTDLPFIGDISDVKTDIFLRK |
| | | DINEETEVIYYPDNVSVDQVILSKNTSEHG |
| | | QLDLLYPSIDSESEILPGENQVFYDNRTQN |
| | | VDYLNSYYYLESQKLSDNVEDFTFTRSIEE |
| | | ALDNSAKVYTYFPTLANKVNAGVQGGLFLM |
| | | WANDVVEDFTTNILRKDTLDKISDVSAIIP |
| | | YIGPALNISNSVRRGNFTEAFAVTGVTILL |
| | | EAFPEFTIPALGAFVIYSKVQERNEIIKTI |
| | | DNCLEQRIKRWKDSYEWMMGTWLSRIITQF |
| | | NNISYQMYDSLNYQAGAIKAKIDLEYKKYS |
| | | GSDKENIKSQVENLKNSLDVKISEAMNNIN |
| | | KFIRECSVTYLFKNMLPKVIDELNEFDRNT |
| | | KAKLINLIDSHNIILVGEVDKLKAKVNNSF |
| | | QNTIPFNIFSYTNNSLLKDIINEYFNNIND |
| | | IVNTSILSIVYKKDDLIDLSRYGAKINIGD |
| | | RVYYDSIDKNQIKLINLESSTIEVILKNAI |
| | | VYNSMYENFSTSFWIKIPKYFSKINLNNEY |
| | | TIINCIENNSGWKVSLNYGEIIWTLQDNKQ |
| | | NIQRVVFKYSQMVNISDYINRWIFVTITNN |
| | | RLTKSKIYINGRLIDQKPISNLGNIHASNK |
| | | IMFKLDGCRDPRRYIMIKYFNLFDKELNEK |
| | | EIKDLYDSQSNSGILKDFWGNYLQYDKPYY |
| | | MLNLFDPNKYVDVNNIGIRGYMYLKGPRGS |
| | | VVTTNIYLNSTLYEGTKFIIKKYASGNEDN |
| | | IVRNNDRVYINVVVKNKEYRLATNASQAGV |
| | | EKILSALEIPDVGNLSQVVVMKSKDDQGIR |
| | | NKCKMNLQDNNGNDIGFIGFHLYDNIAKLV |
| | | ASNWYNRQVGKASRTFGCSWEFIPVDDGWG |
| | | ESSL |
| 117 | BoNT/ | MTWPVKDFNYSDPVNDNDILYLRIPQNKLI |
| | D-A2 | TTPVKAFMITQNIWVIPERFSSDTNPSLSK |
| | chimera | PPRPTSKYQSYYDPSYLSTDEQKDTFLKGI |
| | (wildtype A2 | IKLFKRINERDIGKKLINYLVVGSPFMGDS |
| | receptor | STPEDTFDFTRHTTNIAVEKFENGSWKVTN |
| | binding | IITPSVLIFGPLPNILDYTASLTLQGQQSN |
| | domain) | PSFEGFGTLSILKVAPEFLLTFSDVTSNQS |
| | | SAVLGKSIFCMDPVIALMHELTHSLHQLYG |
| | | INIPSDKRIRPQVSEGFFSQDGPNVQFEEL |
| | | YTFGGLDVEIIPQIERSQLREKALGHYKDI |
| | | AKRLNNINKTIPSSWISNIDKYKKIFSEKY |
| | | NFDKDNTGNFVVNIDKFNSLYSDLTNVMSE |
| | | VVYSSQYNVKNRTHYFSRHYLPVFANILDD |
| | | NIYTIRDGFNLTNKGFNIENSGQNIERNPA |
| | | LQKLSSESVVDLFTKVCLRLTKNSRDDSTC |
| | | IKVKNNRLPYVADKDSISQEIFENKIITDE |
| | | TNVQNYSDNFSLDESILDGQVPINPEIVDP |
| | | LLPNVNMEPLNLPGEEIVFYDDITKYVDYL |
| | | NSYYYLESQKLSNNVENITLTTSVEEALGY |
| | | SNKIYTFLPSLAEKVNKGVQAGLFLNWANE |
| | | VVEDFTTNIMKKDTLDKISDVSVIIPYIGP |
| | | ALNIGNSALRGNFKQAFATAGVAFLLEGFP |
| | | EFTIPALGVFTFYSSIQEREKIIKTIENCL |
| | | EQRVKRWKDSYQWMVSNWLSRITTQFNHIN |
| | | YQMYDSLSYQADAIKAKIDLEYKKYSGSDK |
| | | ENIKSQVENLKNSLDVKISEAMNNINKFIR |
| | | ECSVTYLFKNMLPKVIDELNKFDLRTKTEL |
| | | INLIDSHNIILVGEVDRLKAKVNESFENTM |
| | | PFNIFSYTNNSLLKDIINEYFNIVNTSILS |
| | | IVYKKDDLIDLSRYGAKINIGDRVYYDSID |
| | | KNQIKLINLESSTIEVILKNAIVYNSMYEN |
| | | FSTSFWIKIPKYFSKINLNNEYTIINCIEN |
| | | NSGWKVSLNYGEIIWTLQDNKQNIQRVVFK |
| | | YSQMVNISDYINRWIFVTITNNRLTKSKIY |
| | | INGRLIDQKPISNLGNIHASNKIMFKLDGC |
| | | RDPRRYIMIKYFNLFDKELNEKEIKDLYDS |
| | | QSNSGILKDFWGNYLQYDKPYYMLNLFDPN |
| | | KYVDVNNIGIRGYMYLKGPRGSVVTTNIYL |
| | | NSTLYEGTKFIIKKYASGNEDNIVRNNDRV |
| | | YINVVVKNKEYRLATNASQAGVEKILSALE |
| | | IPDVGNLSQVVVMKSKDDQGIRNKCKMNLQ |
| | | DNNGNDIGFIGFHLYDNIAKLVASNWYNRQ |
| | | VGKASRTFGCSWEFIPVDDGWGESSL |
| 118 | BoNT/ | MPKINSFNYNDPVNDRTILYIKPGGCQEFY |
| | E-A2 | KSFNIMKNIWIIPERNVIGTTPQDFHPPTS |
| | chimera | LKNGDSSYYDPNYLQSDEEKDRFLKIVTKI |
| | (wildtype A2 | FNRINNNLSGGILLEELSKANPYLGNDNTP |
| | receptor | DNQFHIGDASAVEIKFSNGSQDILLPNVII |
| | binding | MGAEPDLFETNSSNISLRNNYMPSNHRFGS |
| | domain) | IAIVTFSPEYSFRFNDNCMNEFIQDPALTL |
| | | MHELIHSLHGLYGAKGITTKYTITQKQNPL |
| | | ITNIRGTNIEEFLTFGGTDLNIITSAQSND |
| | | IYTNLLADYKKIASKLSKVQVSNPLLNPYK |
| | | DVFEAKYGLDKDASGIYSVNINKENDIFKK |
| | | LYSFTEFDLRTKFQVKCRQTYIGQYKYFKL |
| | | SNLLNDSIYNISEGYNINNLKVNFRGQNAN |
| | | LNPRIITPITGRGLVKKIIRFCKNIVSVKG |
| | | IRKSICIEINNGELFFVASENSYNDDNINT |
| | | PKEIDDTVTSNNNYENDLDQVILNFNSESA |
| | | PGLSDEKLNLTIQNDAYIPKYDSNGTSDIE |
| | | QHDVNELNVFFYLDAQKVPEGENNVNLTSS |
| | | IDTALLEQPKIYTFFSSEFINNVNKPVQAA |
| | | LFVSWIQQVLVDFTTEANQKSTVDKIADIS |
| | | IVVPYIGLALNIGNEAQKGNFKDALELLGA |
| | | GILLEFEPELLIPTILVFTIKSFLGSSDNK |
| | | NKVIKAINNALKERDEKWKEVYSFIVSNWM |
| | | TKINTQFNKRKEQMYQALQNQVNAIKTIIE |
| | | SKYNSYTLEEKNELTNKYDIKQIENELNQK |
| | | VSIAMNNIDRFLTESSISYLMKIINEVKIN |
| | | KLREYDENVKTYLLNYIIQHGSILGESQQE |
| | | LNSMVTDTLNNSIPFKLSSYTDDKILISYF |
| | | NKFFIVNTSILSIVYKKDDLIDLSRYGAKI |
| | | NIGDRVYYDSIDKNQIKLINLESSTIEVIL |
| | | KNAIVYNSMYENFSTSFWIKIPKYFSKINL |
| | | NNEYTIINCIENNSGWKVSLNYGEIIWTLQ |
| | | DNKQNIQRVVFKYSQMVNISDYINRWIFVT |
| | | ITNNRLTKSKIYINGRLIDQKPISNLGNIH |
| | | ASNKIMFKLDGCRDPRRYIMIKYFNLFDKE |
| | | LNEKEIKDLYDSQSNSGILKDFWGNYLQYD |
| | | KPYYMLNLFDPNKYVDVNNIGIRGYMYLKG |
| | | PRGSVVTTNIYLNSTLYEGTKFIIKKYASG |
| | | NEDNIVRNNDRVYINVVVKNKEYRLATNAS |
| | | QAGVEKILSALEIPDVGNLSQVVVMKSKDD |
| | | QGIRNKCKMNLQDNNGNDIGFIGFHLYDNI |
| | | AKLVASNWYNRQVGKASRTFGCSWEFIPVD |
| | | DGWGESSL |
| 119 | BoNT/ | MPVVINSFNYNDPVNDDTILYMQIPYEEKS |
| | F-A2 | KKYYKAFEIMRNVWIIPERNTIGTDPSDFD |
| | chimera | PPASLENGSSAYYDPNYLTTDAEKDRYLKT |
| | (wildtype A2 | TIKLFKRINSNPAGEVLLQEISYAKPYLGN |
| | receptor | EHTPINEFHPVTRTTSVNIKSSTNVKSSII |
| | binding | LNLLVLGAGPDIFENSSYPVRKLMDSGGVY |
| | domain) | DPSNDGFGSINIVTFSPEYEYTFNDISGGY |
| | | NSSTESFIADPAISLAHELIHALHGLYGAR |
| | | GVTYKETIKVKQAPLMIAEKPIRLEEFLTF |
| | | GGQDLNIITSAMKEKIYNNLLANYEKIATR |
| | | LSRVNSAPPEYDINEYKDYFQWKYGLDKNA |
| | | DGSYTVNENKFNEIYKKLYSFTEIDLANKF |
| | | KVKCRNTYFIKYGFLKVPNLLDDDIYTVSE |
| | | GFNIGNLAVNNRGQNIKLNPKIIDSIPDKG |
| | | LVEKIVKFCKSVIPRKGTKAPPRLCIRVNN |
| | | RELFFVASESSYNENDINTPKEIDDTTNLN |
| | | NNYRNNLDEVILDYNSETIPQISNQTLNTL |
| | | VQDDSYVPRYDSNGTSEIEEHNVVDLNVFF |
| | | YLHAQKVPEGETNISLTSSIDTALSEESQV |
| | | YTFFSSEFINTINKPVHAALFISWINQVIR |
| | | DFTTEATQKSTFDKIADISLVVPYVGLALN |
| | | IGNEVQKENFKEAFELLGAGILLEFVPELL |
| | | IPTILVFTIKSFIGSSENKNKIIKAINNSL |
| | | MERETKWKEIYSWIVSNWLTRINTQFNKRK |
| | | EQMYQALQNQVDAIKTVIEYKYNNYTSDER |
| | | NRLESEYNINNIREELNKKVSLAMENIERF |
| | | ITESSIFYLMKLINEAKVSKLREYDEGVKE |
| | | YLLDYISEHRSILGNSVQELNDLVTSTLNN |
| | | SIPFELSSYTNDKILILYFNKLYIVNTSIL |
| | | SIVYKKDDLIDLSRYGAKINIGDRVYYDSI |
| | | DKNQIKLINLESSTIEVILKNAIVYNSMYE |
| | | NFSTSFWIKIPKYFSKINLNNEYTIINCIE |
| | | NNSGWKVSLNYGEIIWTLQDNKQNIQRVVF |
| | | KYSQMVNISDYINRWIFVTITNNRLTKSKI |
| | | YINGRLIDQKPISNLGNIHASNKIMFKLDG |
| | | CRDPRRYIMIKYFNLFDKELNEKEIKDLYD |
| | | SQSNSGILKDFWGNYLQYDKPYYMLNLFDP |
| | | NKYVDVNNIGIRGYMYLKGPRGSVVTTNIY |
| | | LNSTLYEGTKFIIKKYASGNEDNIVRNNDR |
| | | VYINVVVKNKEYRLATNASQAGVEKILSAL |
| | | EIPDVGNLSQVVVMKSKDDQGIRNKCKMNL |
| | | QDNNGNDIGFIGFHLYDNIAKLVASNWYNR |
| | | QVGKASRTFGCSWEFIPVDDGWGESSL |
| 120 | BoNT/ | MPVNIKXFNYNDPINNDDIIMMEPFNDPGP |
| | G-A2 | GTYYKAFRIIDRIWIVPERFTYGFQPDQFN |
| | chimera | ASTGVFSKDVYEYYDPTYLKTDAEKDKFLK |
| | (wildtype A2 | TMIKLFNRINSKPSGQRLLDMIVDAIPYLG |
| | receptor | NASTPPDKFAANVANVSINKKIIQPGAEDQ |
| | binding | IKGLMTNLIIFGPGPVLSDNFTDSMIMNGH |
| | domain) | SPISEGFGARMMIRFCPSCLNVFNNVQENK |
| | | DTSIFSRRAYFADPALTLMHELIHVLHGLY |
| | | GIKISNLPITPNTKEFFMQHSDPVQAEELY |
| | | TFGGHDPSVISPSTDMNIYNKALQNFQDIA |
| | | NRLNIVSSAQGSGIDISLYKQIYKNKYDFV |
| | | EDPNGKYSVDKDKFDKLYKALMFGFTETNL |
| | | AGEYGIKTRYSYFSEYLPPIKTEKLLDNTI |
| | | YTQNEGENIASKNLKTEFNGQNKAVNKEAY |
| | | EEISLEHLVIYRIAMCKPVMYKNTGKSEQC |
| | | IIVNNEDLFFIANKDSFSKDLAKAETIAYN |
| | | TQNNTIENNFSIDQLILDNDLSSGIDLPNE |
| | | NTEPFTNFDDIDIPVYIKQSALKKIFVDGD |
| | | SLFEYLHAQTFPSNIENLQLTNSLNDALRN |
| | | NNKVYTFFSTNLVEKANTVVGASLFVNWVK |
| | | GVIDDFTSESTQKSTIDKVSDVSIIIPYIG |
| | | PALNVGNETAKENFKNAFEIGGAAILMEFI |
| | | PELIVPIVGFFTLESYVGNKGHIIMTISNA |
| | | LKKRDQKWTDMYGLIVSQWLSTVNTQFYTI |
| | | KERMYNALNNQSQAIEKIIEDQYNRYSEED |
| | | KMNINIDFNDIDFKLNQSINLAINNIDDFI |
| | | NQCSISYLMNRMIPLAVKKLKDFDDNLKRD |
| | | LLEYIDTNELYLLDEVNILKSKVNRHLKDS |
| | | IPFDLSLYTKDTILIQVFNNYIIVNTSILS |
| | | IVYKKDDLIDLSRYGAKINIGDRVYYDSID |
| | | KNQIKLINLESSTIEVILKNAIVYNSMYEN |
| | | FSTSFWIKIPKYFSKINLNNEYTIINCIEN |
| | | NSGWKVSLNYGEIIWTLQDNKQNIQRVVFK |
| | | YSQMVNISDYINRWIFVTITNNRLTKSKIY |
| | | INGRLIDQKPISNLGNIHASNKIMFKLDGC |
| | | RDPRRYIMIKYFNLFDKELNEKEIKDLYDS |
| | | QSNSGILKDFWGNYLQYDKPYYMLNLFDPN |
| | | KYVDVNNIGIRGYMYLKGPRGSVVTTNIYL |
| | | NSTLYEGTKFIIKKYASGNEDNIVRNNDRV |
| | | YINVVVKNKEYRLATNASQAGVEKILSALE |
| | | IPDVGNLSQVVVMKSKDDQGIRNKCKMNLQ |
| | | DNNGNDIGFIGFHLYDNIAKLVASNWYNRQ |
| | | VGKASRTFGCSWEFIPVDDGWGESSL |
| 121 | BoNT/ | MPVVINSFNYDDPVNDNTIIYIRPPYYETS |
| | H-A2 | NTYFKAFQIMDNVWIIPERYRLGIDPSLFN |
| | chimera | PPVSLKAGSDGYFDPNYLSTNTEKNKYLQI |
| | (wildtype A2 | MIKLFKRINSKPAGQILLEEIKNAIPYLGN |
| | receptor | SYTQEEQFTTNNRTVSFNVKLANGNIVQQM |
| | binding | ANLIIWGPGPDLTTNKTGGIIYSPYQSMEA |
| | domain) | TPYKDGFGSIMTVEFSPEYATAFNDISIAS |
| | | HSPSLFIKDPALILMHELIHVLHGLYGTYI |
| | | TEYKITPNVVQSYMKVTKPITSAEFLTFGG |
| | | RDRNIVPQSIQSQLYNKVLSDYKRIASRLN |
| | | KVNTATALINIDEFKNLYEWKYQFAKDSNG |
| | | VYSVDLNKFEQLYKKIYSFTEFNLAYEFKI |
| | | KTRLGYLAENFGPFYLPNLLDDSIYTEVDG |
| | | FNIGALSINYQGQNIGSDINSIKKLQGQGV |
| | | VSRVVRLCSNSNTKNSLCITVNNRDLFFIA |
| | | SQESYGENTINTYKEIDDTTTLDPSFEDIL |
| | | DKVILNFNEQVIPQMPNRNVSTDIQKDNYI |
| | | PKYDYNRTDIIDSYEVGRNYNTFFYLNAQK |
| | | FSPNESNITLTSSFDTGLLEGSKVYTFFSS |
| | | DFINNINKPVQALLFIEWVKQVIRDFTTEA |
| | | TKTSTVDKLKDISLVVPYIGLALNIGDEIY |
| | | KQHFAEAVELVGAGLLLEFSPEFLIPTLLI |
| | | FTIKGYLTGSIRDKDKIIKTLDNALNVRDQ |
| | | KWKELYRWVVSKWLTTINTQFNKRKEQMYK |
| | | ALKNQATAIKKIIENKYNNYTTDEKSKIDS |
| | | SYNINEIERTLNEKINLAMKNIEQFITESS |
| | | IAYLINIINNETIQKLKSYDDLVRRYLLGY |
| | | IRNHSSILGNSVEELNSKVNNHLDNGIPFE |
| | | LSSYTNDSLLIRYFNKNYIVNTSILSIVYK |
| | | KDDLIDLSRYGAKINIGDRVYYDSIDKNQI |
| | | KLINLESSTIEVILKNAIVYNSMYENFSTS |
| | | FWIKIPKYFSKINLNNEYTIINCIENNSGW |
| | | KVSLNYGEIIWTLQDNKQNIQRVVFKYSQM |
| | | VNISDYINRWIFVTITNNRLTKSKIYINGR |
| | | LIDQKPISNLGNIHASNKIMFKLDGCRDPR |
| | | RYIMIKYFNLFDKELNEKEIKDLYDSQSNS |
| | | GILKDFWGNYLQYDKPYYMLNLFDPNKYVD |
| | | VNNIGIRGYMYLKGPRGSVVTTNIYLNSTL |
| | | YEGTKFIIKKYASGNEDNIVRNNDRVYINV |
| | | VVKNKEYRLATNASQAGVEKILSALEIPDV |
| | | GNLSQVVVMKSKDDQGIRNKCKMNLQDNNG |
| | | NDIGFIGFHLYDNIAKLVASNWYNRQVGKA |
| | | SRTFGCSWEFIPVDDGWGESSL |
| 122 | BoNT/ | MKLEINKFNYNDPIDGINVITMRPPRHSDK |
| | X-A2 | INKGKGPFKAFQVIKNIWIVPERYNFTNNT |
| | chimera | NDLNIPSEPIMEADAIYNPNYLNTPSEKDE |
| | (wildtype A2 | FLQGVIKVLERIKSKPEGEKLLELISSSIP |
| | receptor | LPLVSNGALTLSDNETIAYQENNNIVSNLQ |
| | binding | ANLVIYGPGPDIANNATYGLYSTPISNGEG |
| | domain) | TLSEVSFSPFYLKPFDESYGNYRSLVNIVN |
| | | KFVKREFAPDPASTLMHELVHVTHNLYGIS |
| | | NRNFYYNFDTGKIETSRQQNSLIFEELLTF |
| | | GGIDSKAISSLIIKKIIETAKNNYTTLISE |
| | | RLNTVTVENDLLKYIKNKIPVQGRLGNFKL |
| | | DTAEFEKKLNTILFVLNESNLAQRFSILVR |
| | | KHYLKERPIDPIYVNILDDNSYSTLEGFNI |
| | | SSQGSNDFQGQLLESSYFEKIESNALRAFI |
| | | KICPRNGLLYNAIYRNSKNYLNNIDLEDKK |
| | | TTSKTNVSYPCSLLNGCIEVENKDLFLISN |
| | | KDSLNDINLSEEKIKPETTVFFKDKLPPQD |
| | | ITLSNYDFTEANSIPSISQQNILERNEELY |
| | | EPIRNSLFEIKTIYVDKLTTFHFLEAQNID |
| | | ESIDSSKIRVELTDSVDEALSNPNKVYSPF |
| | | KNMSNTINSIETGITSTYIFYQWLRSIVKD |
| | | FSDETGKIDVIDKSSDTLAIVPYIGPLLNI |
| | | GNDIRHGDFVGAIELAGITALLEYVPEFTI |
| | | PILVGLEVIGGELAREQVEAIVNNALDKRD |
| | | QKWAEVYNITKAQWWGTIHLQINTRLAHTY |
| | | KALSRQANAIKMNMEFQLANYKGNIDDKAK |
| | | IKNAISETEILLNKSVEQAMKNTEKFMIKL |
| | | SNSYLTKEMIPKVQDNLKNFDLETKKTLDK |
| | | FIKEKEDILGTNLSSSLRRKVSIRLNKNIA |
| | | FDINDIPFSEFDDLINQYKIVNTSILSIVY |
| | | KKDDLIDLSRYGAKINIGDRVYYDSIDKNQ |
| | | IKLINLESSTIEVILKNAIVYNSMYENFST |
| | | SFWIKIPKYFSKINLNNEYTIINCIENNSG |
| | | WKVSLNYGEIIWTLQDNKQNIQRVVFKYSQ |
| | | MVNISDYINRWIFVTITNNRLTKSKIYING |
| | | RLIDQKPISNLGNIHASNKIMFKLDGCRDP |
| | | RRYIMIKYFNLFDKELNEKEIKDLYDSQSN |
| | | SGILKDFWGNYLQYDKPYYMLNLFDPNKYV |
| | | DVNNIGIRGYMYLKGPRGSVVTTNIYLNST |
| | | LYEGTKFIIKKYASGNEDNIVRNNDRVYIN |
| | | VVVKNKEYRLATNASQAGVEKILSALEIPD |
| | | VGNLSQVVVMKSKDDQGIRNKCKMNLQDNN |
| | | GNDIGFIGFHLYDNIAKLVASNWYNRQVGK |
| | | ASRTFGCSWEFIPVDDGWGESSL |
| 123 | BoNT/ | MVTINDLHYSDPIDEDNIINMRIPLYDLEV |
| | EN-A2 | DDQFINHNVPDLKAFQVFPNVWVVPERYTF |
| | chimera | YSTMKNLDAPANPSRSSYYDPTYLQSDAEK |
| | (wildtype A2 | EVFLQQMILLFKRINSTQEGQQFLNLLSRS |
| | receptor | IPVPYESNGDVAMGTTQVIKQMDDKGNVLK |
| | binding | HRRAHIIIYGPGPDLMAKGSKALTKSRETG |
| | domain) | RGCMAEIYFSPMYHKTYSTKLTNKNSLVDK |
| | | SVQEFVPDPAVTLIHELCHGLHALYGIDLG |
| | | NVGSWEFNSNPNSLFSSWFSSKEAVNFEEV |
| | | MTFGGEDVKVIKSEIDKKIPGILNLIKTTV |
| | | EPIINKITDPHDEMLQCLQSKYPSLKGTLG |
| | | QFFFDDTQLEKDIRDLWMVMNETMFAENLK |
| | | ALTRARYLVPKVENIVQVDILSPNVYTIDK |
| | | GFNHLSKGFKGQSVSQSYFRKISALARGAV |
| | | VRACPNPHFSSQRGLSSCIEILEDDLFIMS |
| | | SKDSFTDTDFSEPSVGPVSYKAKKGADTIL |
| | | DSTLSNYDFSKEINFTSTVPIITVEDPLET |
| | | DEDVPVISEDRTVYVDDYTTFHFLEAQKIG |
| | | KEVVPTQTKVVFTTNMEEALFDSKKVYTVF |
| | | ENTASRINEAGTGIANGMMFYQWLKGIVQD |
| | | FTEEATQKDTFDKISDVTMIVPYLGNILNI |
| | | GNDIRKGDFMGAVELGGVTILLEAIPELTL |
| | | PVLIGLTIIEDELEKEQVSQTVYNVLDKRD |
| | | EKWEEVYGFVKQQWWWMVHTQFETRILHAY |
| | | QALNHQVEAIKANMTYQLANYRGNQEDKEL |
| | | LEKAIDDTLQSLYYAVDQAMHNIKRFLIQS |
| | | SKSYLLNQMLPKTKEQLLAFDQQTLRNVND |
| | | FINKNQGVLGESLAKDLKKKVEKRLTSLPV |
| | | FNLEDLPISEFEDLIHSHEIDIQDSEVLNI |
| | | GVNNIVNTSILSIVYKKDDLIDLSRYGAKI |
| | | NIGDRVYYDSIDKNQIKLINLESSTIEVIL |
| | | KNAIVYNSMYENFSTSFWIKIPKYFSKINL |
| | | NNEYTIINCIENNSGWKVSLNYGEIIWTLQ |
| | | DNKQNIQRVVFKYSQMVNISDYINRWIFVT |
| | | ITNNRLTKSKIYINGRLIDQKPISNLGNIH |
| | | ASNKIMFKLDGCRDPRRYIMIKYFNLFDKE |
| | | LNEKEIKDLYDSQSNSGILKDFWGNYLQYD |
| | | KPYYMLNLFDPNKYVDVNNIGIRGYMYLKG |
| | | PRGSVVTTNIYLNSTLYEGTKFIIKKYASG |
| | | NEDNIVRNNDRVYINVVVKNKEYRLATNAS |
| | | QAGVEKILSALEIPDVGNLSQVVVMKSKDD |
| | | QGIRNKCKMNLQDNNGNDIGFIGFHLYDNI |
| | | AKLVASNWYNRQVGKASRTFGCSWEFIPVD |
| | | DGWGESSL |

### REFERENCES

1. Schiavo, G., Matteoli, M. & Montecucco, C. Neurotoxins affecting neuroexocytosis. Physiol Rev 80, 717-766 (2000).
2. Dong, M., Masuyer, G. & Stenmark, P. Botulinum and Tetanus Neurotoxins. Annu

Rev Biochem 88, 811-837 (2019).
3. Johnson, E. A. Clostridial toxins as therapeutic agents: benefits of nature's most toxic proteins. Annu Rev Microbiol 53, 551-575 (1999).
4. Aoki, K. R. Botulinum toxin: a successful therapeutic protein. Curr Med Chem 11, 3085-3092 (2004).
5. Montecucco, C. & Molgo, J. Botulinal neurotoxins: revival of an old killer. Curr Opin Pharmacol 5, 274-279 (2005).
6. Lange, O., Bigalke, H., Dengler, R., Wegner, F., deGroot, M. & Wohlfarth, K. Neutralizing antibodies and secondary therapy failure after treatment with botulinum toxin type A: much ado about nothing? Clin Neuropharmacol 32, 213-218 (2009).
7. Chapman, M. A., Barron, R., Tanis, D. C., Gill, C. E. & Charles, P. D. Comparison of botulinum neurotoxin preparations for the treatment of cervical dystonia. Clin Ther 29, 1325-1337 (2007).
8. Cote, T. R., Mohan, A. K., Polder, J. A., Walton, M. K. & Braun, M. M. Botulinum toxin type A injections: adverse events reported to the US Food and Drug Administration in therapeutic and cosmetic cases. J Am Acad Dermatol 53, 407-415 (2005).
9. Dong, M., Tepp, W. H., Liu, H., Johnson, E. A. & Chapman, E. R. Mechanism of botulinum neurotoxin B and G entry into hippocampal neurons. J Cell Biol 179, 1511-1522 (2007).
10. Peng, L., Tepp, W. H., Johnson, E. A. & Dong, M. Botulinum neurotoxin D uses synaptic vesicle protein SV2 and gangliosides as receptors. PLoS Pathog 7, e1002008 (2011).
11. Dong, M., Richards, D. A., Goodnough, M. C., Tepp, W. H., Johnson, E. A. & Chapman, E. R. Synaptotagmins I and II mediate entry of botulinum neurotoxin B into cells. J

Cell Biol 162, 1293-1303 (2003).
12. Nishiki, T., Kamata, Y., Nemoto, Y., Omori, A., Ito, T., Takahashi, M. & Kozaki, S. Identification of protein receptor for Clostridium botulinum type B neurotoxin in rat brain synaptosomes. J Biol Chem 269, 10498-10503 (1994).
13. Rummel, A., Karnath, T., Henke, T., Bigalke, H. & Binz, T. Synaptotagmins I and II act as nerve cell receptors for botulinum neurotoxin G. J Biol Chem 279, 30865-30870 (2004).
14. Peng, L., Berntsson, R. P., Tepp, W. H., Pitkin, R. M., Johnson, E. A., Stenmark, P. & Dong, M. Botulinum neurotoxin D-C uses synaptotagmin I/II as receptors and human synaptotagmin II is not an effective receptor for type B, D-C, and G toxins. J Cell Sci (2012).
15. Jin, R., Rummel, A., Binz, T. & Brunger, A. T. Botulinum neurotoxin B recognizes its protein receptor with high affinity and specificity. Nature 444, 1092-1095 (2006).
16. Chai, Q., Arndt, J. W., Dong, M., Tepp, W. H., Johnson, E. A., Chapman, E. R. & Stevens, R. C. Structural basis of cell surface receptor recognition by botulinum neurotoxin B. Nature 444, 1096-1100 (2006).
17. Benoit, R. M., Frey, D., Hilbert, M., Kevenaar, J. T., Wieser, M. M., Stirnimann, C. U., McMillan, D., Ceska, T., Lebon, F., Jaussi, R., Steinmetz, M. O., Schertler, G. F., Hoogenraad, C. C., Capitani, G. & Kammerer, R. A. Structural basis for recognition of synaptic vesicle protein 2C by botulinum neurotoxin A. Nature 505, 108-111 (2014).
18. Dong, M., Liu, H., Tepp, W. H., Johnson, E. A., Janz, R. & Chapman, E. R. Glycosylated SV2A and SV2B mediate the entry of botulinum neurotoxin E into neurons. Mol Biol Cell 19, 5226-5237 (2008).
19. Dong, M., Yeh, F., Tepp, W. H., Dean, C., Johnson, E. A., Janz, R. & Chapman, E. R. SV2 is the protein receptor for botulinum neurotoxin A. Science 312, 592-596 (2006).
20. Mahrhold, S., Rummel, A., Bigalke, H., Davletov, B. & Binz, T. The synaptic vesicle protein 2C mediates the uptake of botulinum neurotoxin A into phrenic nerves. FEBS Lett 580, 2011-2014 (2006).
21. Rummel, A., Hafner, K., Mahrhold, S., Darashchonak, N., Holt, M., Jahn, R., Beermann, S., Karnath, T., Bigalke, H. & Binz, T. Botulinum neurotoxins C, E and F bind gangliosides via a conserved binding site prior to stimulation-dependent uptake with botulinum neurotoxin F utilising the three isoforms of SV2 as second receptor. J Neurochem 110, 1942-1954 (2009).
22. Fu, Z., Chen, C., Barbieri, J. T., Kim, J. J. & Baldwin, M. R. Glycosylated SV2 and gangliosides as dual receptors for botulinum neurotoxin serotype F. Biochemistry 48, 5631-5641 (2009).
23. Montecucco, C. How do tetanus and botulinum toxins bind to neuronal membranes? TIBS 11, 314-317 (1986).
24. Peck, M. W., Smith, T. J., Anniballi, F., Austin, J. W., Bano, L., Bradshaw, M., Cuervo, P., Cheng, L. W., Derman, Y., Dorner, B. G., Fisher, A., Hill, K. K., Kalb, S. R., Korkeala, H., Lindstrom, M., Lista, F., Luquez, C., Mazuet, C., Pirazzini, M., Popoff, M. R., Rossetto, O., Rummel, A., Sesardic, D., Singh, B. R. & Stringer, S. C. Historical Perspectives and Guidelines for Botulinum Neurotoxin Subtype Nomenclature. Toxins (Basel) 9(2017).
25. Pier, C. L., Chen, C., Tepp, W. H., Lin, G., Janda, K. D., Barbieri, J. T., Pellett, S. & Johnson, E. A. Botulinum neurotoxin subtype A2 enters neuronal cells faster than subtype A1.

FEBS Lett 585, 199-206 (2011).
26. Yao, G., Zhang, S., Mahrhold, S., Lam, K. H., Stern, D., Bagramyan, K., Perry, K., Kalkum, M., Rummel, A., Dong, M. & Jin, R. N-linked glycosylation of SV2 is required for binding and uptake of botulinum neurotoxin A. Nat Struct Mol Biol 23, 656-662 (2016).
27. Barash, J. R. & Arnon, S. S. A novel strain of Clostridium botulinum that produces type B and type H botulinum toxins. J Infect Dis 209, 183-191 (2014).
28. Zhang, S., Masuyer, G., Zhang, J., Shen, Y., Lundin, D., Henriksson, L., Miyashita, S. I., Martinez-Carranza, M., Dong, M. & Stenmark, P. Identification and characterization of a novel botulinum neurotoxin. Nat Commun 8, 14130 (2017).
29. Popp, M. W., Antos, J. M., Grotenbreg, G. M., Spooner, E. & Ploegh, H. L. Sortagging: a versatile method for protein labeling. Nat Chem Biol 3, 707-708 (2007).
30. Aoki, K. R. A comparison of the safety margins of botulinum neurotoxin serotypes A, B, and F in mice. Toxicon 39, 1815-1820 (2001).
31. Arnon, S. S., Schechter, R., Inglesby, T. V., Henderson, D. A., Bartlett, J. G., Ascher, M. S., Eitzen, E., Fine, A. D., Hauer, J., Layton, M., Lillibridge, S., Osterholm, M. T., O'Toole, T., Parker, G., Perl, T. M., Russell, P. K., Swerdlow, D. L. & Tonat, K. Botulinum toxin as a biological weapon: medical and public health management. Jama 285, 1059-1070 (2001).
32. Montal, M. Botulinum neurotoxin: a marvel of protein design. Annu Rev Biochem 79, 591-617 (2010).
33. Rossetto, O., Pirazzini, M. & Montecucco, C. Botulinum neurotoxins: genetic, structural and mechanistic insights. Nat Rev Microbiol 12, 535-549 (2014).
34. Pirazzini, M., et al. Thioredoxin and its reductase are present on synaptic vesicles, and their inhibition prevents the paralysis induced by botulinum neurotoxins. Cell Rep 8, 1870-1878 (2014).
35. Pirazzini, M., et al. The thioredoxin reductase—Thioredoxin redox system cleaves the interchain disulphide bond of botulinum neurotoxins on the cytosolic surface of synaptic vesicles. Toxicon 107, 32-36 (2015).
36. JaHN, R. & Scheller, R. H. SNAREs—engines for membrane fusion. Nat Rev Mol Cell Biol 7, 631-643 (2006).
37. Sudhof, T. C. & Rothman, J. E. Membrane fusion: grappling with SNARE and SM proteins. Science 323, 474-477 (2009).
38. Gu, S., et al. Botulinum neurotoxin is shielded by NTNHA in an interlocked complex. Science 335, 977-981 (2012).
39. Lee, K., et al. Molecular basis for disruption of E-cadherin adhesion by botulinum neurotoxin A complex. Science 344, 1405-1410 (2014).
40. Lee, K., et al. Molecular basis for disruption of E-cadherin adhesion by botulinum neurotoxin A complex. Science 344, 1405-1410 (2014).
41. Sugawara, Y., et al. Botulinum hemagglutinin disrupts the intercellular epithelial barrier by directly binding E-cadherin. J Cell Biol 189, 691-700 (2010).
42. Hill, K. K., Xie, G., Foley, B. T. & Smith, T. J. Genetic diversity within the botulinum neurotoxin-producing bacteria and their neurotoxins. Toxicon 107, 2-8 (2015).

What is claimed is:

1. A modified Clostridial botulinum neurotoxin (BoNT) polypeptide comprising a modified receptor binding domain of Clostridial botulinum serotype A1 (BoNT/A1) comprising one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1.

2. The modified BoNT polypeptide of claim 1, wherein the modified receptor binding domain comprises one or more amino acid substitutions at positions corresponding to 954, 955, 957, 1063, 1064, 1025, 1026, 1156, 1232, 1278, 1294, and 1295 in SEQ ID NO: 1.

3. The modified BoNT polypeptide of claim 1 or claim 2, wherein the modified receptor binding domain comprises an amino acid substitution at a position corresponding to 917 in SEQ ID NO: 1, optionally wherein the amino acid substitution corresponds to F917R or F917K in SEQ ID NO: 1.

4. The modified BoNT polypeptide of any one of claims 1-3, wherein the modified receptor binding domain comprises an amino acid substitution at a position corresponding to 953 in SEQ ID NO: 1, optionally wherein the amino acid substitution corresponds to F953H or F953Y in SEQ ID NO: 1.

5. The modified BoNT polypeptide of any one of claims 1-4, wherein the modified receptor binding domain comprises an amino acid substitution at a position corresponding to 954 in SEQ ID NO: 1, optionally wherein the amino acid substitution corresponds to N954S in SEQ ID NO: 1.

6. The modified BoNT polypeptide of any one of claims 1-5, wherein the modified receptor binding domain comprises an amino acid substitution at a position corresponding to 955 in SEQ ID NO: 1, optionally wherein the amino acid substitution corresponds to S955K in SEQ ID NO: 1.

7. The modified BoNT polypeptide of any one of claims 1-6, wherein the modified receptor binding domain comprises an amino acid substitution at a position corresponding to 957 in SEQ ID NO: 1, optionally wherein the amino acid substitution corresponds to S957N, S957Q, or S957Y in SEQ ID NO: 1.

8. The modified BoNT polypeptide of any one of claims 1-7, wherein the modified receptor binding domain comprises an amino acid substitution at a position corresponding to 968 in SEQ ID NO: 1, optionally wherein the amino acid substitution corresponds to M968I in SEQ ID NO: 1.

9. The modified BoNT polypeptide of any one of claims 1-8, wherein the modified receptor binding domain comprises an amino acid substitution at a position corresponding to 1025 in SEQ ID NO: 1, optionally wherein the amino acid substitution corresponds to N1025T in SEQ ID NO: 1.

10. The modified BoNT polypeptide of any one of claims 1-9, wherein the modified receptor binding domain comprises an amino acid substitution at a position corresponding to 1026 in SEQ ID NO: 1, optionally wherein the amino acid substitution corresponds to N1026K in SEQ ID NO: 1.

11. The modified BoNT polypeptide of any one of claims 1-10, wherein the modified receptor binding domain comprises an amino acid substitution at a position corresponding to 1052 in SEQ ID NO: 1, optionally wherein the amino acid substitution corresponds to N1052K in SEQ ID NO: 1.

12. The modified BoNT polypeptide of any one of claims 1-11, wherein the modified receptor binding domain comprises an amino acid substitution at a position corresponding to 1062 in SEQ ID NO: 1, optionally wherein the amino acid substitution corresponds to D1062E in SEQ ID NO: 1.

13. The modified BoNT polypeptide of any one of claims 1-12, wherein the modified receptor binding domain comprises an amino acid substitution at a position corresponding to 1063 in SEQ ID NO: 1, optionally wherein the amino acid substitution corresponds to T1063P in SEQ ID NO: 1.

14. The modified BoNT polypeptide of any one of claims 1-13, wherein the modified receptor binding domain comprises an amino acid substitution at a position corresponding to 1064 in SEQ ID NO: 1, optionally wherein the amino acid substitution corresponds to H1064R or H1064Q in SEQ ID NO: 1.

15. The modified BoNT polypeptide of any one of claims 1-14, wherein the modified receptor binding domain comprises an amino acid substitution at a position corresponding to 1065 in SEQ ID NO: 1, optionally wherein the amino acid substitution corresponds to R1065N in SEQ ID NO: 1.

16. The modified BoNT polypeptide of any one of claims 1-15, wherein the modified receptor binding domain comprises an amino acid substitution at a position corresponding to 1066 in SEQ ID NO: 1, optionally wherein the amino acid substitution corresponds to Y1066R or Y1066K in SEQ ID NO: 1.

17. The modified BoNT polypeptide of any one of claims 1-16, wherein the modified receptor binding domain comprises an amino acid substitution at a position corresponding to 1145 in SEQ ID NO: 1, optionally wherein the amino acid substitution corresponds to T1145Y in SEQ ID NO: 1.

18. The modified BoNT polypeptide of any one of claims 1-17, wherein the modified receptor binding domain comprises an amino acid substitution at a position corresponding to 1156 in SEQ ID NO: 1, optionally wherein the amino acid substitution corresponds to R1156M or R1156I in SEQ ID NO: 1.

19. The modified BoNT polypeptide of any one of claims 1-18, wherein the modified receptor binding domain comprises an amino acid substitution at a position corresponding to 1232 in SEQ ID NO: 1, optionally wherein the amino acid substitution corresponds to T1232R or T1232K in SEQ ID NO: 1.

20. The modified BoNT polypeptide of any one of claims 1-19, wherein the modified receptor binding domain comprises an amino acid substitution at a position corresponding to 1272 in SEQ ID NO: 1, optionally wherein the amino acid substitution corresponds to E1272G in SEQ ID NO: 1.

21. The modified BoNT polypeptide of any one of claims 1-20, wherein the modified receptor binding domain comprises an amino acid substitution at a position corresponding to 1278 in SEQ ID NO: 1, optionally wherein the amino acid substitution corresponds to L1278F, L1278Y, or L1278W in SEQ ID NO: 1.

22. The modified BoNT polypeptide of any one of claims 1-21, wherein the modified receptor binding domain comprises an amino acid substitution at a position corresponding to 1288 in SEQ ID NO: 1, optionally wherein the amino acid substitution corresponds to D1288E or D1288N in SEQ ID NO: 1.

23. The modified BoNT polypeptide of any one of claims 1-22, wherein the modified receptor binding domain comprises an amino acid substitution at a position corresponding to 1289 in SEQ ID NO: 1, optionally wherein the amino acid substitution corresponds to D1289Y in SEQ ID NO: 1.

24. The modified BoNT polypeptide of any one of claims 1-23, wherein the modified receptor binding domain comprises an amino acid substitution at a position corresponding to 1292 in SEQ ID NO: 1, optionally wherein the amino acid substitution corresponds to G1292R or G1292K in SEQ ID NO: 1.

25. The modified BoNT polypeptide of any one of claims 1-24, wherein the modified receptor binding domain comprises an amino acid substitution at a position corresponding to 1294 in SEQ ID NO: 1, optionally wherein the amino acid substitution corresponds to R1294S or R1294T in SEQ ID NO: 1.

26. The modified BoNT polypeptide of any one of claims 1-25, wherein the modified receptor binding domain comprises an amino acid substitution at a position corresponding to 1295 in SEQ ID NO: 1, optionally wherein the amino acid substitution corresponds to P1295S or P1295T in SEQ ID NO: 1.

27. The modified BoNT polypeptide of any one of claims 1-26, wherein the modified receptor binding domain comprises an amino acid sequence that is at least 80% identical to the amino acid sequence of SEQ ID NO: 51-85

28. The modified BoNT polypeptide of claim 27, wherein the modified receptor binding domain comprises the amino acid sequence of any one of SEQ ID NOs:51-85.

29. The modified BoNT polypeptide of any one of claims 1-28, further comprising a protease domain and a translocation domain from BoNT/A1.

30. The modified BoNT polypeptide of claim 29, wherein the modified BoNT polypeptide comprises an amino acid sequence that is at least 80% identical to the amino acid sequence of SEQ ID NO: 3-37.

31. The modified BoNT polypeptide of claim 30, wherein the modified BoNT polypeptide comprises the amino acid sequence of SEQ ID NO: 3-37.

32. The modified BoNT polypeptide of any one of claims 1-31, further comprising a protease domain and a translocation domain from a second BoNT, optionally wherein the second BoNT is of serotype B, C, D, E, F, G, H, X, or En.

33. The modified BoNT polypeptide of claim 32, wherein the modified BoNT polypeptide comprises an amino acid sequence that is at least 80% identical to the amino acid sequence of any one of SEQ ID NO: 97-105 fused to any one of SEQ ID NO: 51-85.

34. The modified BoNT polypeptide of claim 33, wherein the modified BoNT polypeptide comprises the amino acid sequence of any one of SEQ ID NO: 97-105 fused to any one of SEQ ID NO: 51-85.

35. A modified Clostridial Botulinum neurotoxin (BoNT) polypeptide comprising a modified receptor binding domain of Clostridial Botulinum serotype A2 (BoNT/A2) comprising one or more amino acid substitutions at positions corresponding to 915, 923, 1090, 1103, 1117, 1156, 1170, 1227, 1254, 1255, or 1256 in SEQ ID NO: 2.

36. The modified BoNT polypeptide of claim 35, wherein the modified receptor binding domain comprises an amino acid substitution at a position corresponding to 915 in SEQ ID NO: 2, optionally wherein the amino acid substitution corresponds to K915Q in SEQ ID NO: 2.

37. The modified BoNT polypeptide of claim 35 or claim 36, wherein the modified receptor binding domain comprises an amino acid substitution at a position corresponding to 923 in SEQ ID NO: 2, optionally wherein the amino acid substitution corresponds to T923K in SEQ ID NO: 2.

38. The modified BoNT polypeptide of anyone of claims 35-37, wherein the modified receptor binding domain comprises an amino acid substitution at a position corresponding to 1090 in SEQ ID NO: 2, optionally wherein the amino acid substitution corresponds to S1090N in SEQ ID NO: 2.

39. The modified BoNT polypeptide of anyone of claims 35-38, wherein the modified receptor binding domain comprises an amino acid substitution at a position corresponding to 1103 in SEQ ID NO: 2, optionally wherein the amino acid substitution corresponds to N1103D in SEQ ID NO: 2.

40. The modified BoNT polypeptide of anyone of claims 35-39, wherein the modified receptor binding domain comprises an amino acid substitution at a position corresponding to 1117 in SEQ ID NO: 2, optionally wherein the amino acid substitution corresponds to F1117Y in SEQ ID NO: 2.

41. The modified BoNT polypeptide of anyone of claims 35-40, wherein the modified receptor binding domain comprises an amino acid substitution at a position corresponding to 1156 in SEQ ID NO: 2, optionally wherein the amino acid substitution corresponds to E1156M in SEQ ID NO: 2.

42. The modified BoNT polypeptide of anyone of claims 35-41, wherein the modified receptor binding domain comprises an amino acid substitution at a position corresponding to 1170 in SEQ ID NO: 2, optionally wherein the amino acid substitution corresponds to E1170K in SEQ ID NO: 2.

43. The modified BoNT polypeptide of anyone of claims 35-42, wherein the modified receptor binding domain comprises an amino acid substitution at a position corresponding to 1227 in SEQ ID NO: 2, optionally wherein the amino acid substitution corresponds to D1227N in SEQ ID NO: 2.

44. The modified BoNT polypeptide of anyone of claims 35-43, wherein the modified receptor binding domain comprises an amino acid substitution at a position corresponding to 1254 in SEQ ID NO: 2, optionally wherein the amino acid substitution corresponds to L1254Q in SEQ ID NO: 2.

45. The modified BoNT polypeptide of anyone of claims 35-44, wherein the modified receptor binding domain comprises an amino acid substitution at a position corresponding to 1255 in SEQ ID NO: 2, optionally wherein the amino acid substitution corresponds to Y1255F in SEQ ID NO: 2.

46. The modified BoNT polypeptide of anyone of claims 35-45, wherein the modified receptor binding domain comprises an amino acid substitution at a position corresponding to 1256 in SEQ ID NO: 2, optionally wherein the amino acid substitution corresponds to D1256N in SEQ ID NO: 2.

47. The modified BoNT polypeptide of any one of claims 35-46, wherein the modified receptor binding domain comprises an amino acid sequence that is at least 80% identical to the amino acid sequence of SEQ ID NO: 86-96.

48. The modified BoNT polypeptide of claim 47, wherein the modified receptor binding domain comprises the amino acid sequence of any one of SEQ ID NOs: 86-96.

49. The modified BoNT polypeptide of any one of claims 35-48, further comprising a protease domain and a translocation domain from BoNT/A1.

50. The modified BoNT polypeptide of claim 49, wherein the modified BoNT polypeptide comprises an amino acid sequence that is at least 80% identical to the amino acid sequence of SEQ ID NO: 38-48.

51. The modified BoNT polypeptide of claim 50, wherein the modified BoNT polypeptide comprises the amino acid sequence of SEQ ID NO: 38-48.

52. The modified BoNT polypeptide of any one of claims 35-48, further comprising a protease domain and a translocation domain from a second BoNT, optionally wherein the second BoNT is of serotype B, C, D, E, F, G, H, X, or En.

53. The modified BoNT polypeptide of claim 52, wherein the modified BoNT polypeptide comprises an amino acid sequence that is at least 80% identical to the amino acid sequence of any one of SEQ ID NO: 97-105 fused to any one of SEQ ID NO: 86-96.

54. The modified BoNT polypeptide of claim 53, wherein the modified BoNT polypeptide comprises the amino acid sequence of any one of SEQ ID NO: 97-105 fused to any one of SEQ ID NO: 86-96.

55. A nucleic acid molecule comprising a polynucleotide encoding a modified BoNT polypeptide of any one of claims 1-34 and 35-54.

56. A nucleic acid vector comprising the nucleic acid molecule of claim 55.

57. A cell comprising the nucleic acid molecule of claim 55 or the nucleic acid vector of claim 56.

58. A cell expressing the modified BoNT polypeptide of any one of claims 1-34 and 35-54.

59. A method of producing a modified BoNT polypeptide, the method comprising the steps of culturing the cell of claim 57 or claim 58 under conditions wherein the modified BoNT polypeptide is produced.

60. The method of claim 59, further comprising recovering the modified BoNT polypeptide from the culture.

61. A pharmaceutical composition comprising the modified BoNT polypeptide of any one of claims 1-34 and 35-54.

62. The pharmaceutical composition of claim 61, further comprising a pharmaceutically acceptable excipient.

63. A kit comprising a pharmaceutical composition of claim 61 or claim 62 and directions for therapeutic administration of the pharmaceutical composition.

64. A method of treating a condition, the method comprising administering a therapeutically effective amount of the modified BoNT polypeptide of any one of claims 1-34 and 35-54, or the pharmaceutical composition of or claim 61 or 62 to a subject to treat the condition.

65. The method of claim 64, wherein the condition is associated with overactive neurons or glands.

66. The method of claim 65, wherein the condition is selected from the group consisting of: spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite, depression.

67. The method of claim 64 or 65, wherein the condition is not associated with unwanted neuronal activity.

68. The method of claim 67, wherein the condition is selected from the group consisting of: psoriasis, allergy, haemophagocytic lymphohistiocytosis, and alcoholic pancreatic disease.

69. The method of any one of any one of claims 64-68, wherein the administering is via injection to where unwanted neuronal activity is present.

70. The modified BoNT polypeptide of any one of claims 1-34 and 35-54, or the pharmaceutical composition of claim 61 or 62, for use in treating a condition associated with unwanted neuronal activity.

71. The modified BoNT polypeptide of any one of claims 1-34 and 35-54, or the pharmaceutical composition of claim 61 or 62, for use in medicine.

72. The modified BoNT polypeptide of any one of claims 1-34 and 35-54, or the pharmaceutical composition of claim 61 or 62, for cosmetic use.
